(19) United States
(12) Patent Application Publication
(10) Pub. No.: US 2004/0152750 A1
(43) Pub. Date: Aug. 5, 2004

(54) Novel glucagon antagonists

(76) Inventors: Janos Tibor Kodra, Copenhagen (DK); Carsten Behrens, Kobenhavn (DK); Peter Madsen, Bagsvaerd (DK); Anker Steen Jorgensen, Kobenhavn (DK); Inger Thoger Christensen, Lyngby (DK)

Correspondence Address: NOVO NORDISK PHARMACEUTICALS, INC, 100 COLLEGE ROAD WEST, PRINCETON, NY, 08540, US

(21) Appl. No.: 10/735,986

(22) Filed: Dec. 15, 2003

Related U.S. Application Data
(60) Provisional application No. 60/438,131, filed on Jan. 6, 2003.

(30) Foreign Application Priority Data
Dec. 20, 2002 (DK) PA 2002 01967

(51) Int. Cl. A61K 31/41; A61K 31/198

(52) U.S. Cl. 514/381; 514/563; 548/256; 562/450

(57) ABSTRACT

Novel compounds that act to antagonize the action of the glucagon peptide hormone on the glucagon receptor. More particularly, it relates to glucagon antagonists or inverse agonists.

### CROSS-REFERENCE TO RELATED APPLICATIONS

This application claims priority under 35 U.S.C. 119 of Danish application no. PA 2002 01967 filed Dec. 20, 2002 and U.S. application No. 60/438,131 filed Jan. 6, 2003, the contents of each of which are fully incorporated herein by reference.

### FIELD OF THE INVENTION

The present invention relates to agents that act to antagonize the action of the glucagon peptide hormone on the glucagon receptor. More particularly, it relates to glucagon antagonists or inverse agonists.

### BACKGROUND OF THE INVENTION

Glucagon is a key hormonal agent that, in co-operation with insulin, mediates homeostatic regulation of the amount of glucose in the blood. Glucagon primarily acts by stimu-lating certain cells (mostly liver cells) to release glucose when blood glucose levels fall. The action of glucagon is opposite to that of insulin, which stimulates cells to take up and store glucose whenever blood glucose levels rise. Both glucagon and insulin are peptide hormones.

Glucagon is produced in the alpha islet cells of the pancreas and insulin in the beta islet cells. Diabetes mellitus is a common disorder of glucose metabolism. The disease is characterized by hyperglycemia and may be classified as type 1 diabetes, the insulindependent form, or type 2 diabetes, which is non-insulin-dependent in character. Subjects with type 1 diabetes are hyperglycemic and hypoinsulinemic, and the conventional treatment for this form of the disease is to provide insulin. However, in some patients with type 1 or type 2 diabetes, absolute or relative elevated glucagon levels have been shown to contribute to the hyperglycemic state. Both in healthy control animals as well as in animal models of type 1 and type 2 diabetes, removal of circulating glucagon with selective and specific antibodies has resulted in reduction of the glycemic level. These studies suggest that glucagon suppression or an action that antagonizes glucagon could be a useful adjunct to conventional treatment of hyperglycemia in diabetic patients. The action of glucagon can be suppressed by providing an antagonist or an inverse agonist, ie substances that inhibit or prevent glucagon-induced responses. The antagonist can be peptidic or non-peptidic in nature.

Native glucagon is a 29 amino acid peptide having the sequence: His-Ser-Glu-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH

Glucagon exerts its action by binding to and activating its receptor, which is part of the Glucagon-Secretin branch of the 7-transmembrane G-protein coupled receptor family. The receptor functions by activating the adenylyl cyclase second messenger system and the result is an increase in cAMP levels.

Several publications disclose peptides that are stated to act as glucagon antagonists. Probably, the most thoroughly characterized antagonist is DesHis¹[Glu⁹]-glucagon amide (Unson et al., Peptides 10, 1171 (1989); Post et al., Proc. Natl. Acad. Sci. USA 90, 1662 (1993)). Other antagonists are DesHis¹, Phe⁶[Glu⁹]-glucagon amide (Azizh et al., Bioorganic & Medicinal Chem. Lett. 16, 1849 (1995)) and NLeu⁹, Ala^{11,16}-glucagon amide (Unson et al., J. Biol. Chem. 269 (17), 12548 (1994)).

Peptide antagonists of peptide hormones are often quite potent. However, they are generally known not to be orally available because of degradation by physiological enzymes, and poor distribution in vivo. Therefore, orally available non-peptide antagonists of peptide hormones are generally preferred. Among the non-peptide glucagon antagonists, a quinoxaline derivative, (2-styryl-3-[3-(dimethylamino)propylm ethylamino]-6,7-d ichloroquinoxaline was found to displace glucagon from the rat liver receptor (Collins, J. L. et al., Bioorganic and Medicinal Chem. Lett. 2(9):915-918 (1992)). WO 94/14426 (The Wellcome Foundation Limited) discloses use of skyrin, a natural product comprising a pair of linked 9,10-anthracenedione groups, and its synthetic analogues, as glucagon antagonists. U.S. Pat. No. 4,359,474 (Sandoz) discloses the glucagon inhibiting properties of 1-phenyl pyrazole derivatives. U.S. Pat. No. 4,374,130 (Sandoz) discloses substituted disilacyclohexanes as glucagon inhibiting agents. WO 98/04528 (Bayer Corporation) discloses substituted pyridines and biphenyls as glucagon antagonists. U.S. Pat. No. 5,776,954 (Merck & Co., Inc.) discloses substituted pyridyl pyrroles as glucagon antagonists and WO 98/21957, WO 98/22108, WO 98/22109 and U.S. Pat. No. 5,880,139 (Merck & Co., Inc.) disclose 2,4-diaryl-5-pyridylimidazoles as glucagon antagonists. Furthermore, WO 97/16442 and U.S. Pat. No. 5,837,719 (Merck & Co., Inc.) disclose 2,5-substituted aryl pyrroles as glucagon antagonists. WO 98/24780, WO 98/24782, WO 99/24404 and WO 99/32448 (Amgen Inc.) disclose substituted pyrimidinone and pyridone compounds and substituted pyrimidine compounds, respectively, which are stated to possess glucagon antagonistic activity. Madsen et al. (J. Med. Chem. 1998 (41) 5151-7) discloses a series of 2-(benzimidazol-2-ylthio)-1-(3,4-dihydroxyphenyl)-1-ethanones as competitive human glucagon receptor antagonists. WO 99/01423 and WO 00/39088 (Novo Nordisk A/S) disclose different series of alkylidene hydrazides as glucagon antagonists/inverse agonists. WO 00/69810, WO 02/00612, WO 02/40444, WO 02/40445 and WO 02/40446 (Novo Nordisk A/S) disclose further classes of glucagon antagonists. A. L. Handlon et al. (GlaxoSmithKline) recently published aminooxadiazoles as glucagon antagonists (226^{th} ACS National Meeting, Division of Medicinal Chemistry (poster no. 164)). Furthermore, WO 03/064404 (Dainippon Pharm Co, LTD) discloses 2-furancarboxylic acid hydrazides as glucagon antagonists.

These known glucagon antagonists differ structurally from the present compounds.

### DEFINITIONS

The following is a detailed definition of the terms used to describe the compounds of the invention:

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl and the like.

The term “C₁₋₆-alkoxy” as used herein refers to the radical —O—C₁₋₆-alkyl wherein C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “C₃₋₈-cycloalkyl” as used herein represents a saturated, carbocyclic group having from 3 to 8 carbon atoms. Representative examples are cyclopropyl, cyclobutyl, cyclopentyl, cyclohexyl, cycloheptyl, cyclooctyl and the like.

The term “C₄₋₈-cycloalkenyl” as used herein represents a non-aromatic, carbocyclic group having from 4 to 8 carbon atoms containing one or two double bonds. Representative examples are 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl, 2-cycloheptenyl, 3-cycloheptenyl, 2-cyclooctenyl, 1,4-cyclooctadienyl and the like.

The term “heterocyclyl” as used herein represents a non-aromatic 3 to 10 membered ring containing one or more heteroatoms selected from nitrogen, oxygen and sulfur and optionally containing one or two double bonds. Representative examples are pyrrolidinyl, piperidyl, piperazinyl, morpholinyl, thiomorpholinyl, aziridinyl, tetrahydrofuranyl and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl, indanyl and the like.

The term “arylene” as used herein is intended to include divalent, carbocyclic, aromatic ring systems such as 6 membered monocyclic and 9 to 14 membered bi- and tricyclic, divalent, carbocyclic, aromatic ring systems. Representative examples are phenylene, bi-phenylene, naphthylene, anthracenylene, phenanthrenylene, fluorenylene, indenylene, azulenylene and the like. Arylene is also intended to include the partially hydrogenated derivatives of the ring systems enumerated above. Non-limiting examples of such partially hydrogenated derivatives are 1,2,3,4-tetrahydronaphthylene, 1,4-dihydronaphthylene and the like.

The term “aryloxy” as used herein denotes a group —O-aryl, wherein aryl is as defined above.

The term “aroyl” as used herein denotes a group —C(O)-aryl, wherein aryl is as defined above.

The term C₁-alkanoyl as used herein denotes a group —C(O)—C₁₋₆-alkyl, wherein C₁₋₆-alkyl is as defined above.

The term “heteroaryl” as used herein is intended to include aromatic, heterocyclic ring systems containing one or more heteroatoms selected from nitrogen, oxygen and sulfur such as 5 to 7 membered monocyclic and 8 to 14 membered bi- and tricyclic aromatic, heterocyclic ring systems containing one or more heteroatoms selected from nitrogen, oxygen and sulfur. Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyi, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the ring systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term “heteroarylene” as used herein is intended to include divalent, aromatic, heterocyclic ring systems containing one or more heteroatoms selected from nitrogen, oxygen and sulfur such as 5 to 7 membered monocyclic and 8 to 14 membered bi- and tricyclic aromatic, heterocyclic ring systems containing one or more heteroatoms selected from nitrogen, oxygen and sulfur. Representative examples are furylene, thienylene, pyrrolylene, oxazolylene, thiazolylene, imidazolylene, isoxazolylene, isothiazolylene, 1,2,3-triazolylene, 1,2,4-triazolylene, pyranylene, pyridylene, pyridazinylene, pyrimidinylene, pyrazinylene, 1,2,3-triazinylene, 1,2,4-triazinylene, 1,3,5-triazinylene, 1,2,3-oxadiazolylene, 1,2,4-oxadiazolylene, 1,2,5-oxadiazolylene, 1,3,4-oxadiazolylene, 1,2,3-thiadiazolylene, 1,2,4-thiadiazolylene, 1,2,5-thiadiazolylene, 1,3,4-thiadiazolylene, tetrazolylene, thiadiazinylene, indolylene, isoindolylene, benzofurylene, benzothienylene, indazolylene, benzimidazolylene, benzthiazolylene, benzisothiazolylene, benzoxazolylene, benzisoxazolylene, purinylene, quinazolinylene, quinolizinylene, quinolinylene, isoquinolinylene, quinoxalinylene, naphthyridinylene, pteridinylene, carbazolylene, azepinylene, diazepinylene, acridinylene and the like. Heteroaryl is also intended to include the partially hydrogenated derivatives of the ring systems enumerated above. Non-limiting examples of such partially hydrogenated derivatives are 2,3-dihydrobenzofuranylene, pyrrolinylene, pyrazolinylene, indolinylene, oxazolidinylene, oxazolinylene, oxazepinylene and the like.

“Aryl-C₁₋₆-alkyl”, “heteroaryl-C₁₋₆-alkyl”, “aryl-C₂₋₆-alkenyl” etc. mean C₁₋₆-alkyl or C₂₋₆-alkenyl as defined above, substituted by an aryl or heteroaryl as defined above, for example:

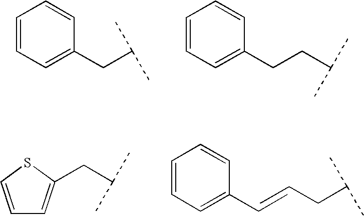

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The invention thus provides a compound of formula (I):

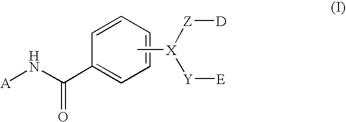

wherein

A is

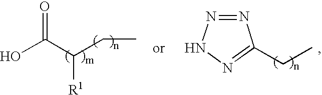

m is 0 or 1,

n is 0, 1, 2 or 3,

with the proviso that m and n must not both be 0,

R¹ is hydrogen, fluoro or —(CH₂)₀—OR²,

o is 0 or 1,

R² is hydrogen, C₁₋₆-alkyl, C₁₋₆-alkanoyl, aryl or aryl-C₁₋₆-alkyl,

X is N, CH or C with a double bond to one substituent,

Z is —CR³R⁴—, —(C═O)—(NR⁵)—(C₁₋₁-alkyl)_{K}—, —(C═O)—O—(C₁₋₁-alkyl)_{K}—, —(C═O)—(C₁₋₆-alkyl)_{K}—, —(C₁₋₆-alkyl)_{K}(C═O)—O—, —(C═O)—O—(C₂₋₆-alkenyl)_{K}—, —(C═O)—(C₂₋₆-alkenyl)_{K}—, —(C₁₋₆-alkenyl)_{K}(C═O)—O—

wherein k is 0 or 1,

R³, R⁴ and R⁵ are independently selected from hydrogen, C₁-alkyl or aryl,

Y is —C₁₋₆-alkyl)ₛ—(C═O)—(C₁₋₆-alkyl)ₜ—, —C₁₋₆-alkenyl)ₛ—(C═O)—(C₁₋₆-alkyl)ₜ—, —C₁₋₆-alkyl—, —C₂₋₆-alkenyl-, or —CR⁶R⁷—

wherein s and t independently are 0 or 1;

wherein R⁶, R⁷ and R⁸ independently are selected from hydrogen, C₁₋₁-alkyl and aryl;

The linkers Y and Z are to be understood as optionally attached in either direction,

D is aryl or heteroaryl, which may optionally be substituted with one or more substituents R¹⁶, R¹⁷ R¹⁸ R¹⁹, R²⁰ and R²¹, wherein

R¹⁶, R¹⁷, R¹⁸ and R¹⁹ independently are

hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR²², —NR²²R²³, —SR²², —NR²²S(O)₂R²³, —S(O)₂NR²²R²³—S(O)NR²²R²³—S(O)R²², —S(O)₂R²², —C(O)NR²²R²³, —OC(O)NR R²³, —NR²²C(O)R²³—CH₂C(O)NR²²R²³—OCH₂C(O)NR²²R²³, —CH₂OR²², —CH₂NR²²R²³. —OC(O)R²², —C(O)R²² or —C(O)OR²²

C₁₋₆-alkyl, C₂₋₆-alkenyl or C₂₋₆-alkynyl,

which may optionally be substituted with one or more substituents selected from halogen, —CN, —CF₃, —OCF₃, —NO₂, —OR²², —NR²²R²³ and C₁₋₆-alkyl,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl, aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,

of which the cyclic moieties optionally may be substituted with one or more substituents selected from halogen, —C(O)OR²², —CN, —CF₃, —OCF₃, —NO₂, —OR²², —NR²²R²³ and C₁-alkyl,

R²² and R²³ independently are hydrogen, C₁₋₆-alkyl, aryl-C₁₋₆-alkyl or aryl, or R²² and R²³ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R¹⁶ to R¹⁹ when placed in adjacent positions together may form a bridge —(CR²⁴R²⁵)ₐ—O—(CR²⁶R²⁷)_{c}—O—,

a is 0, 1 or 2,

c is 1 or 2,

R²⁴, R²⁵, R²⁶ and R²⁷ independently are hydrogen, C₁₋₁-alkyl or fluoro,

R²⁰ and R²¹ independently are hydrogen, C₁₋₆-alkyl, C₃₋₈-cycloalkyl or C₃₋₈-cycloalkyl-C₁₋₆-alkyl,

E is

C₃₋₈-cycloalkyl or C₄₋₈-cycloalkenyl, which may optionally be substituted with one or two substituents R²⁸ and R²⁹, which are independently selected from

hydrogen, halogen, —CN, —CF₃, —OR³³, —NR³³R³⁴, C₁₋₆-alkyl, C₃₋₈-cycloalkyl, C₄₋₈-cycloalkenyl, heteroaryl and aryl,

wherein the heteroaryl and aryl groups optionally may be substituted with one or more substituents selected from halogen, —CN, —CF₃, —NO₂, —OR³³, —NR³³R³⁴ and C₁-alkyl,

R³³ and R³⁴ independently are hydrogen or C₁₋₆-alkyl,

or R³³ and R³⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

aryl, heteroaryl, aryl-C₂₋₆-alkenyl or aryl-C₂₋₆-alkynyl, of which the cyclic moieties may optionally be substituted with one to three substitutents R³⁰, R³¹ and R³², which are independently selected from

hydrogen, halogen, —CHF₂, —CF₃, —OCF₃, —OCHF₂, —OCH₂CF₃, —OCF₂CHF₂, —SCF₃, —OR³⁵, —NR³⁵R³⁵, —SR³⁵, —S(O)R³⁵, —S(O)₂R³⁵, —C(O)NR³⁵R³⁶, —OC(O)NR³⁵R³⁵, —NR³⁵C(O)R³⁶, —OCH₂C(O)NR³⁵R³⁶, —C(O)R³⁵ and —C(O)OR³⁵,

C₁₋₆-alkyl, C₂₋₆-alkenyl and C₂₋₆-alkynyl,

which may optionally be substituted with one or more substituents selected from halogen, —CN, —CF₃, —OCF₃, —SCF₃, —NO₂, —OR, —NR³⁵R³⁶ and C₁₋₆-alkyl,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl, aryl, aryloxy, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl and heteroaryl-C₂₋₆-alkynyl,

of which the cyclic moieties optionally may be substituted with one or more substituents selected from halogen, —CN, —CF₃, —OCF₃, —SCF₃, —NO₂, —OR³⁵, —NR³⁵R³⁶ and C₁₋₆-alkyl,

wherein R³⁵ and R³⁶ independently are hydrogen, C₁₋₆-alkyl or aryl,

or R³⁵ and R³⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the substituents R³⁰, R³¹ and R³² when attached to the same ring carbon atom or different ring carbon atoms together may form a radical —O—(CH₂)ₜ—CR³⁷R³⁸—(CH₂)ₗ—O—, —(CH₂)ₜ—CR³⁷R³⁸—(CH₂)ₗ— or —S—(CH₂)ₜ—CR³⁷R³⁸—(CH₂)ₗ—S—,

t and l independently are 0, 1, 2, 3, 4 or 5,

R³⁷ and R³⁸ independently are hydrogen or C₁₋₆-alkyl,

as well as any diastereomer or enantiomer or tautomeric form thereof including mixtures of these or a pharmaceutically acceptable salt thereof.

In an embodiment of the invention A is

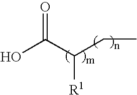

wherein m, n and R⁴ are as defined in claim 1.

In another embodiment A is

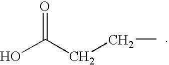

In another embodiment A is

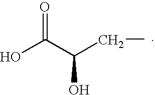

In another embodiment A is

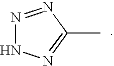

In an embodiment of the invention D is

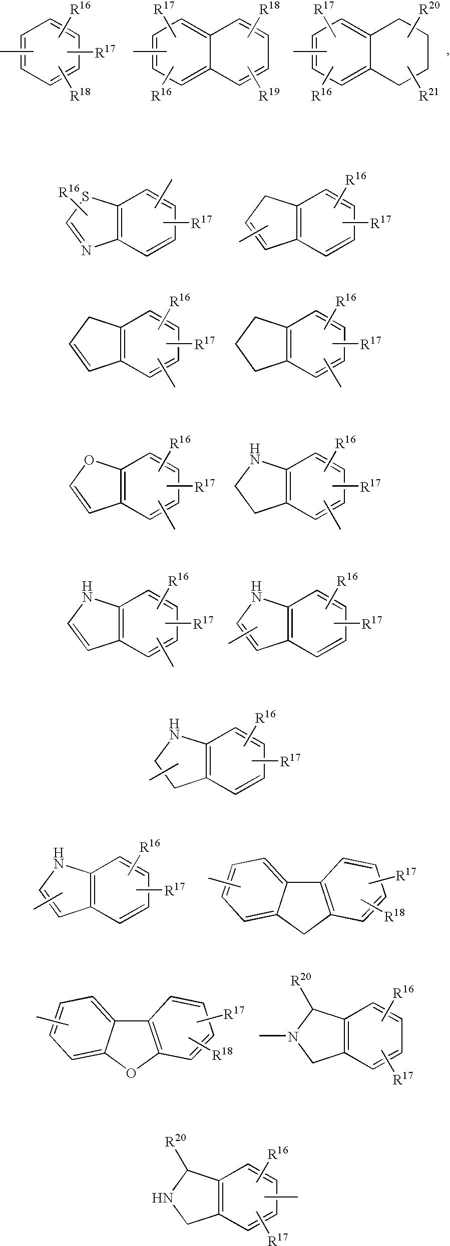

wherein R¹⁶, R¹⁷, R¹⁸, R¹⁹, R²⁰ and R²¹ are as defined in claim 1.

In another embodiment D is

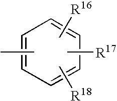

wherein R¹⁶, R¹⁷ and R¹⁸ are as defined in claim 1.

In another embodiment R¹⁶, R¹⁷ and R¹⁸ independently are

hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR²², —NR²²R²³, —SR²², —NR²²S(O)₂R²³, —S(O)₂NR²²R²³ S(O)NR²²R²³—S(O)R²², —S(O)₂R²², —C(O)NR²²R²³, —OC(O)NR²²R²³, —NR²²C(O)R²³, —CH₂C(O)NR²²R²³, —OCH₂C(O)NR²²R²³, —CH₂OR²², —CH₂NR²²R²³—OC(O)R²², —C(O)R²² or —C(O)OR²²

C₁₋₆-alkyl, which may optionally be substituted with one or more substituents selected from fluoro, —CN, —CF₃, —OCF₃, —OR²² and —NR²²R²³,

C₃₋₈-cycloalkyl, which may optionally be substituted with one or more substituents selected from fluoro, —C(O)OR²⁴, —CN, —CF₃, —OCF₃, —OR²², —NR²²R²³ and C₁ alkyl,

aryl or aryloxy, which may optionally be substituted with one or more substituents selected from halogen, —C(O)OR²², —CN, —CF₃, —OCF₃, —NO₂, —OR²², —NR²²R²³ and C₁₋₆-alkyl,

R²² and R²³ independently are hydrogen, C₁₋₆-alkyl, aryl-C₁₋₆-alkyl or aryl, or R²² and R²³ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R¹⁶ to R¹⁸ when placed in adjacent positions together may form a bridge —(CR²⁴R²⁵)ₐ—O—(CR²⁶R²⁷)_{c}—O—,

a is 0, 1 or 2,

c is 1 or 2,

R²⁴, R²⁵, R²⁶ and R²⁷ independently are hydrogen, C₁₋₆-alkyl or fluoro.

In another embodiment R¹⁶, R¹⁷ and R¹⁸ independently are

hydrogen, halogen, CN, —CF₃, —OCF₃, —SCF₃, —S(O)C₁₋₆-alkyl-, —C(O)C₁₋₆-alkyl-, C₁₋₆-alkyl, C₁₋₆-alkoxy, phenyl, cyclopentyl, cyclohexyl or phenoxy,

or two of the groups R¹⁶ to R¹⁸ when placed in adjacent positions together may form a bridge —O—(CF₂)₂—O—, —CF₂—O—CF₂—O— or —O—CH₂—O—.

In another embodiment E is

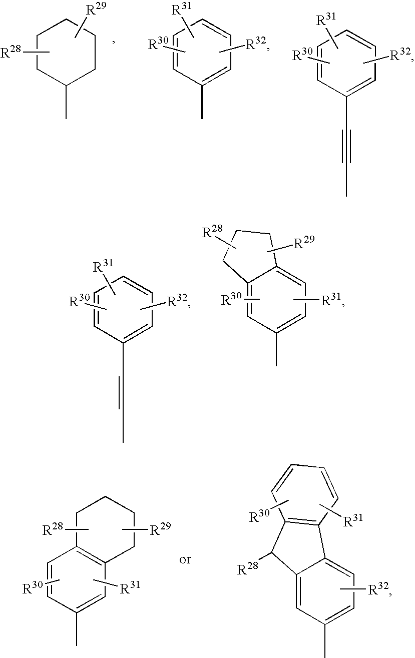

wherein R²⁸ R²⁹, R³⁰ R³¹ and R³² are as defined in claim 1.

In another embodiment E is

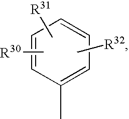

wherein R³⁰, R³′ and R³² are as defined in claim 1.

In another embodiment R³⁰, R³¹ and R³² independently are

hydrogen, halogen, —OCF₃, —CF₃, —OCHF₂ or —CF₃,

C₁₋₆-alkyl, which may optionally be substituted with one or more substituents selected from fluoro, —CN, —CF₃, —OCF₃, —OR³⁵ and —NR³⁵R³⁶,

C₃₋₈-cycloalkyl or C₄₋₈-cycloalkenyl, which may optionally be substituted with one or more substituents selected from fluoro, —CN, —CF₃, —OCF₃, —OR³⁵, —NR³⁵R³⁶ and C₁₋₆-alkyl,

aryl, heteroaryl, aryloxy or aryl-C₁₋₆-alkoxy, of which the aryl moieties may optionally be substituted with one or more substituents selected from halogen, —CN, —CF₃, —OCF₃, —NO₂, —R³⁵, —NR³⁵R³⁶ and C₁₋₆-alkyl,

R³⁵ and R³⁶ independently are hydrogen, C₁₋₆-alkyl or aryl,

or R³⁵ and R³⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

In another embodiment R³⁰, R³¹ and R³² independently are

hydrogen, halogen, —OCF₃, —OCHF₂, —SCF₃, or —CF₃,

C₁₋₆-alkyl, which may optionally be substituted with one or more substituents selected from fluoro, —CN, —CF₃, —OCF₃, —oR³⁵ and —NR³⁵R³⁶,

cyclohexyl or cyclohex-1-enyl, which may optionally be substituted with one or more substituents selected from fluoro, —CN, —CF₃, —OCF₃, —OR³⁵, —NR³⁵R³⁶ and C₁₋₆-alkyl,

phenyl which may optionally be substituted with one or more substitutents selected from halogen, —CN, —CF₃, —OCF₃, —NO₂, —OR³⁵, —NR³⁵R³⁶ and C₁₋₆-alkyl,

phenoxy or benzyloxy, of which the phenyl moieties may optionally be substituted with one or more substituents selected from halogen, —CN, —CF₃, —OCF₃, —NO₂, —OR³⁵, —NR³⁵R³⁶ and C₁₋₆-alkyl,

thiadiazolyl,

R³⁵ and R³⁶ independently are hydrogen or C₁₋₆-alkyl.

In another embodiment R³⁰ and R³² are both hydrogen, and R³¹ is different from hydrogen.

In another embodiment Y is —C═O—, —CH₂—.

In another embodiment Z is —CH₂—, —(C═O)—(NH), —(C═O)—O — or —C═O)—CH₂—.

In another embodiment the compound as above has an IC50 value of no greater than 5 μM as determined by the Glucagon Binding Assay (I) or Glucagon Binding Assay (II) disclosed herein.

In another embodiment the compound has an IC₅₀ value of less than 1 μM, preferably of less than 500 nM and even more preferred of less than 100 nM as determined by the Glucagon Binding Assay (I) or Glucagon Binding Assay (II) disclosed herein.

In another embodiment the compound is an agent useful for the treatment of an indication selected from the group consisting of hyperglycemia, IGT, type 2 diabetes, type 1 diabetes, dyslipidemia and obesity.

The compounds of the present invention may be chiral, and it is intended that any enantiomers, as separated, pure or partially purified enantiomers or racemic mixtures thereof are included within the scope of the invention.

Furthermore, when a double bond or a fully or partially saturated ring system or more than one center of asymmetry or a bond with restricted rotability is present in the molecule diastereomers may be formed. It is intended that any diastereomers, as separated, pure or partially purified diastereomers or mixtures thereof are included within the scope of the invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts, benzatine or benzatine derivatives and the like.

Also intended as pharmaceutically acceptable acid addition salts are the hydrates, which the present compounds, are able to form.

Furthermore, the pharmaceutically acceptable salts comprise basic amino acid salts such as lysine, arginine and ornithine.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming pharmacologically active substances. In general, such prodrugs will be functional derivatives of the compounds of the general formula (I), which are readily convertible in vivo into the required compound of the formula (I). Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds according to the present invention act to antagonize the action of glucagon and are accordingly useful for the treatment of disorders and diseases in which such an antagonism is beneficial.

The compounds according to the present invention preferably have an IC₅₀ value of no greater than 5 μM, more preferably of less than 1 μM, even more preferred of less than 500 nM, such as of less than 100 nM as determined by the Glucagon Binding Assay (I) or Glucagon Binding Assay (II) disclosed herein.

Accordingly, the present compounds may be applicable for the treatment of hyperglycemia, IGT (impaired glucose tolerance), insulin resistance syndromes, syndrome X, type 1 diabetes, type 2 diabetes, hyperlipidemia, dyslipidemia, hypertriglyceridemia, hyperlipoproteinemia, hypercholesterolemia, arteriosclerosis including atherosclerosis, glucagonomas, acute pancreatitis, cardiovascular diseases, hypertension, cardiac hypertrophy, gastrointestinal disorders, obesity, diabetes as a consequence of obesity, diabetic dyslipidemia, etc.

Furthermore, they may be applicable as diagnostic agents for identifying patients having a defect in the glucagon receptor, as a therapy to increase gastric acid secretions and to reverse intestinal hypomobility due to glucagon administration.

They may also be useful as tool or reference molecules in labelled form eg radio-labelled in binding assays to identify new glucagon antagonists.

Accordingly, in a further aspect the invention relates to a compound according to the invention for use as a medicament.

The invention also relates to pharmaceutical compositions comprising, as an active ingredient, at least one compound according to the invention together with one or more pharmaceutically acceptable carriers or excipients.

The pharmaceutical composition is preferably in unit dosage form comprising from about 0.05 mg to about 1000 mg, preferably from about 0.1 mg to about 500 mg and especially preferred from about 0.5 mg to about 200 mg of the compound according to the invention.

Furthermore, the invention relates to the use of a compound according to the invention for the preparation of a pharmaceutical composition for the treatment of a disorder or disease, wherein a glucagon antagonistic action is beneficial.

The invention also relates to a method for the treatment of disorders or diseases, wherein a glucagon antagonistic action is beneficial the method comprising administering to a subject in need thereof an effective amount of a compound according to the invention.

In one embodiment, the present compounds are used for the preparation of a medicament for the treatment of any glucagon-mediated conditions and diseases.

In another embodiment, the present compounds are used for the preparation of a medicament for the treatment of hyperglycemia.

In yet another embodiment, the present compounds are used for the preparation of a medicament for lowering blood glucose in a mammal. The present compounds are effective in lowering the blood glucose, both in the fasting and the postprandial stage.

In yet another embodiment, the present compounds are used for the preparation of a pharmaceutical composition for the treatment of IGT.

In still another embodiment, the present compounds are used for the preparation of a pharmaceutical composition for the treatment of type 2 diabetes.

In yet another embodiment, the present compounds are used for the preparation of a pharmaceutical composition for the delaying or prevention of the progression from IGT to type 2 diabetes.

In yet another embodiment, the present compounds are used for the preparation of a pharmaceutical composition for the delaying or prevention of the progression from non-insulin requiring type 2 diabetes to insulin requiring type 2 diabetes.

In a further embodiment, the present compounds are used for the preparation of a pharmaceutical composition for the treatment of type 1 diabetes. Such treatment is normally accompanied by insulin therapy.

In still a further embodiment, the present compounds are used for the preparation of a pharmaceutical composition for the treatment of obesity.

In yet a further embodiment, the present compounds are used for the preparation of a pharmaceutical composition for the treatment of disorders of the lipid metabolism, such as dyslipidemia.

In still a further embodiment, the present compounds are used for the preparation of a pharmaceutical composition for the treatment of an appetite regulation or energy expenditure disorder.

In a further aspect of the invention, treatment of a patient with the present compounds is combined with diet and/or exercise.

In yet a further aspect of the invention, the present compounds are administered in combination with one or more further active substances in any suitable ratio(s). Such further active agents may be selected from antidiabetic agents, antihyperlipidemic agents, antiobesity agents, antihypertensive agents and agents for the treatment of complications resulting from or associated with diabetes.

Suitable antidiabetic agents include insulin, insulin analogues and derivatives such as those disclosed in EP 792 290 (Novo Nordisk A/S), eg N^{εB29}-tetradecanoyl des (B30) human insulin, EP 214 826 and EP 705 275 (Novo Nordisk A/S), eg Asp^{B28} human insulin, U.S. Pat. No. 5,504,188 (Eli Lilly), eg Lys^{B28} Pro^{B29} human insulin, EP 368 187 (Aventis), eg Lantus®, all of which are incorporated herein by reference, GLP-1 and GLP-1 derivatives such as those disclosed in WO 98/08871 (Novo Nordisk A/S), which is incorporated herein by reference, as well as orally active hypoglycemic agents.

The orally active hypoglycemic agents include imidazolines, sulphonylureas, biguamides, meglitinides, oxadiazolidinediones, thiazolidinediones, α-glucosidase inhibitors, glucagon antagonists, GLP-1 agonists, agents acting on the ATP-dependent potassium channel of the β-cells, eg potassium channel openers such as those disclosed in WO 97/26265, WO 99/03861 and WO 00/37474 (Novo Nordisk A/S), all of which are incorporated herein by reference, or nateglinide or potassium channel blockers such as BTS-67582, insulin sensitizers, insulin secretagogues, DPP-IV (dipeptidyl peptidase-IV) inhibitors, PTPase inhibitors, inhibitors of hepatic enzymes involved in stimulation of gluconeogenesis and/or glycogenolysis, glucose uptake modulators, activators of glucokinase (GK) such as those disclosed in WO 00/58293, WO 01/44216, WO 01/83465, WO 01/83478, WO 01/85706, WO 01/85707 and WO 02/08209 (Hoffman-La Roche), which are incorporated herein by reference, GSK-3 (glycogen synthase kinase-3) inhibitors, compounds modifying the lipid metabolism such as antihyperlipidemic agents and antilipidemic agents, compounds lowering food intake, PPAR (peroxisome proliferator-activated receptor) and RXR (retinoid X receptor) agonists such as ALRT-268, LG-1268 or LG-1069.

In one embodiment, the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lys^{B29}-(Nε(γ-glutamyl-N^{α}litocholyl) des (B30) human insulin, Lantus, or a mix-preparation comprising one or more of these.

In a further embodiment, the present compounds are administered in combination with a sulphonylurea, eg tolbutamide, chlorpropamide, tolazamide, glibenclamide, glyburide, glipizide, glimepride or glicazide.

In another embodiment, the present compounds are administered in combination with a biguamide, eg mefformin.

In yet another embodiment, the present compounds are administered in combination with a meglitinide, eg repaglinide or nateglinide.

In still another embodiment, the present compounds are administered in combination with a thiazolidinedione insulin sensitizer, eg troglitazone, ciglitazone, pioglitazone, rosiglitazone, isaglitazone, darglitazone, englitazone, CS-011/Cl-1037 or T174 or the compounds disclosed in WO 97/41097, WO 97/41119, WO 97/41120, WO 00/41121 and WO 98/45292 (Dr. Reddy's Research Foundation).

In still another embodiment, the present compounds may be administered in combination with an insulin sensitizer such as GI 262570, YM-440, MCC-555, JTT-501, AR-H039242, KRP-297, GW-409544, CRE-16336, AR-H049020, LY510929, LY465608, MBX-102, CLX-0940, GW-501516, tesaglitazar (AZ 242) or the compounds disclosed in WO 99/19313, WO 00/50414, WO 00/63191, WO 00/63192, WO 00/63193 such as ragaglitazar (NN 622 or (−)DRF 2725) (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S).

In a further embodiment, the present compounds are administered in combination with an α-glucosidase inhibitor, eg voglibose, emiglitate, miglitol or acarbose.

In another embodiment, the present compounds are administered in combination with an agent acting on the ATP-dependent potassium channel of the β-cells, eg tolbutamide, chlorpropamide, tolazamide, glibenclamide, glyburide, glipizide, glicazide, BTS-67582, repaglinide or nateglinide.

In still another embodiment, the present compounds are administered in combination with an antihyperlipidemic agent or antilipidemic agent, eg cholestyramine, colestipol, clofibrate, gemfibrozil, lovastatin, pravastatin, simvastatin, probucol or dextrothyroxine.

In another aspect of the invention, the present compounds are administered in combination with more than one of the above-mentioned compounds, eg in combination with metformin and a sulphonylurea such as glibenclamide or glyburide; a sulphonylurea and acarbose; metformin and a meglitinide such as repaglinide; acarbose and mefformin; a sulfonylurea, metformin and troglitazone; a sulfonylurea, mefformin and pioglitazone; a sulfonylurea, mefformin and an insulin sensitizer such as disclosed in WO 00/63189 or WO 97/41097; a meglitinide such as repaglinide, metformin and troglitazone; a meglitinide such as repaglinide, mefformin and pioglitazone; a meglitinide such as repaglinide, metformin and an insulin sensitizer such as disclosed in WO 00/63189 or WO 97/41097; insulin and a sulfonylurea; insulin and a meglitinide such as repaglinide; insulin and mefformin; insulin, metformin and a meglitinide such as repaglinide; insulin, metformin and a sulfonylurea; insulin and troglitazone; insulin and pioglitazone; insulin and an insulin sensitizer such as such as disclosed in WO 00/63189 or WO 97/41097; insulin and lovastatin; an insulin analogue or derivative, metformin and a meglitinide such as repaglinide; an insulin analogue or derivative, mefformin and a sulfonylurea; an insulin analogue or derivative and troglitazone; an insulin analogue or derivative and pioglitazone; an insulin analogue or derivative and an insulin sensitizer such as disclosed in WO 00/63189 or WO 97/41097; an insulin analogue or derivative and lovastatin; etc.

Furthermore, the compounds according to the invention may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, H3 histamine antagonists, TNF (tumor necrosis factor) modulators, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 orAZ-40140, MSH (melanocytestimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators or TR β agonists.

In another embodiment, the antiobesity agent is dexamphetamine or amphetamine.

In another embodiment, the antiobesity agent is fenfluramine or dexfenfluramine.

In still another embodiment, the antiobesity agent is sibutramine.

In a further embodiment, the antiobesity agent is orlistat.

In another embodiment, the antiobesity agent is mazindol or phentermine.

Furthermore, the present compounds may be administered in combination with one or more antihypertensive agents. Examples of antihypertensive agents are P-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19th Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracistemal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is a base addition salt of a compound having the utility of a free acid. When a compound of the formula (I) contains a free acid such salts are prepared in a conventional manner by treating a solution or suspension of a free acid of the formula (I) with a chemical equivalent of a pharmaceutically acceptable base. Representative examples are mentioned above.

For parenteral administration, solutions of the novel compounds of the formula (I) in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

| | | |
|---|---|---|
| Core: | | |
| Active compound (as free compound or salt thereof) | | 5.0 mg |
| Lactosum Ph. Eur. | | 67.8 mg |
| Cellulose, microcryst. (Avicel) | | 31.4 mg |
| Amberlite ® IRP88* | | 1.0 mg |
| Magnesii stearas Ph. Eur. | | q.s. |
| Coating: | | |
| Hydroxypropyl methylcellulose | approx. | 9 mg |
| Mywacett 9-40 T** | approx. | 0.9 mg |

*Polacrillin potassium NF, tablet disintegrant, Rohm and Haas.
**Acylated monoglyceride used as plasticizer for film coating.

If desired, the pharmaceutical composition of the invention may comprise the compound of the formula (I) in combination with further pharmacologically active substances such as those described in the foregoing.

### EXAMPLES

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of the glucagon antagonists of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

Some of the NMR data shown in the following examples are only selected data.

In the examples the following terms are intended to have the following, general meanings:

DABCO: 1,4-diazabicyclo[2.2.2.]octane

DCM: dichloromethane, methylenechloride

DCP: 1,2-dichloropropane

DIC: diisopropylcarbodiimide

DIPEA: N,N-diisopropylethylamine

DMAP: 4-N,N-dimethylaminopyridine

DMF: N,N-dimethylformamide

DMSO: dimethyl sulphoxide

EDAC: ethyl dimethylaminopropyl carbodiimid hydrochloride

Fmoc: 9-fluorenylmethyloxycarbonyl

HOBt: 1-hydroxybenzotriazole

MeOH: methanol

NMP: N-methyl-2-pyrrolidinone

THF: tetrahydrofuran

TFA: trifluoroacetic acid

HPLC-MS (Method A)

The following instrumentation was used:

Sciex API 150 Single Quadropole mass spectrometer

Hewlett Packard Series 1100 G1312A Bin pump

Gilson 215 micro injector

Hewlett Packard Series 1100 G1315A DAD diode array detector

Sedex 55 evaporative light scattering detector

A Valco column switch with a Valco actuator controlled by timed events from the pump.

The Sciex Sample control software running on a Macintosh Power G3 computer was used for the instrument control and data acquisition.

The HPLC pump was connected to two eluent reservoirs containing:

A: Acetonitrile containing 0.05% TFA

B: Water containing 0.05% TFA

The requirements for the samples are that they contain approximately 500 μg/ml of the compound to be analysed in an acceptable solvent such as methanol, ethanol, acetonitrile, THF, water and mixtures thereof. (High concentrations of strongly eluting solvents will interfere with the chromatography at low acetonitrile concentrations.)

The analysis was performed at room temperature by injecting 20 μl of the sample solution on the column, which was eluted with a gradient of acetonitrile in 0.05% TFA The eluate from the column was passed through a flow splitting T-connector, which passed approximately 20 μl/min through approx. 1 m 75μ fused silica capillary to the API interface of API 150 spectrometer.

The remaining 1.48 ml/min was passed through the UV detector and to the ELS detector.

During the LC-analysis the detection data were acquired concurrently from the mass spectrometer, the UV detector and the ELS detector.

The LC conditions, detector settings and mass spectrometer settings used for the different methods are given in the following table.

| Column | Waters X-terra C18 5μ 3 mm × 50 mm id | | | |
|---|---|---|---|---|
| Gradient | 5%-90% acetonitrile in 0.05% TFA linearly during 7.5 min at | | | |
| | 1.5 ml/min | | | |
| Detection | UV: 214 nm | | ELS: 40° C. | |
| MS | Experiment: | Start: 100 amu | Stop: 800 amu | Step: |
| | | | | 0.2 amu |
| | Dwell: | 0.571 msec | | |

| | |
|---|---|
| Method: | Scan 284 times = 9.5 min |

HPLC-MS (Method B)

The following instrumentation was used:

Hewlett Packard series 1100 G1312A Bin Pump

Hewlett Packard series 1100 Column compartment

Hewlett Packard series 1100 G1315A DAD diode array detector

Hewlett Packard series 1100 MSD

The instrument was controlled by HP Chemstation software.

The HPLC pump was connected to two eluent reservoirs containing:

A: 0.01% TFA in water

B: 0.01% TFA in acetonitrile

The analysis was performed at 40° C. by injecting an appropriate volume of the sample (preferably 1 μL) onto the column, which was eluted with a gradient of acetonitrile.

The HPLC conditions, detector settings and mass spectrometer settings used are given in the following table.

| | |
|---|---|
| Column | Waters Xterra MS C-18 × 3 mm id |
| Gradient | 10%-100% acetonitrile lineary during 7.5 min at 1.0 ml/min |
| Detection | UV: 210 nm (analog output from DAD) |
| MS | lonisation mode: API-ES |
| | Scan 100-1000 amu step 0.1 amu |

General Procedure A:

General procedure (A) may be used for solid phase preparation of compounds of general formula (Ia):

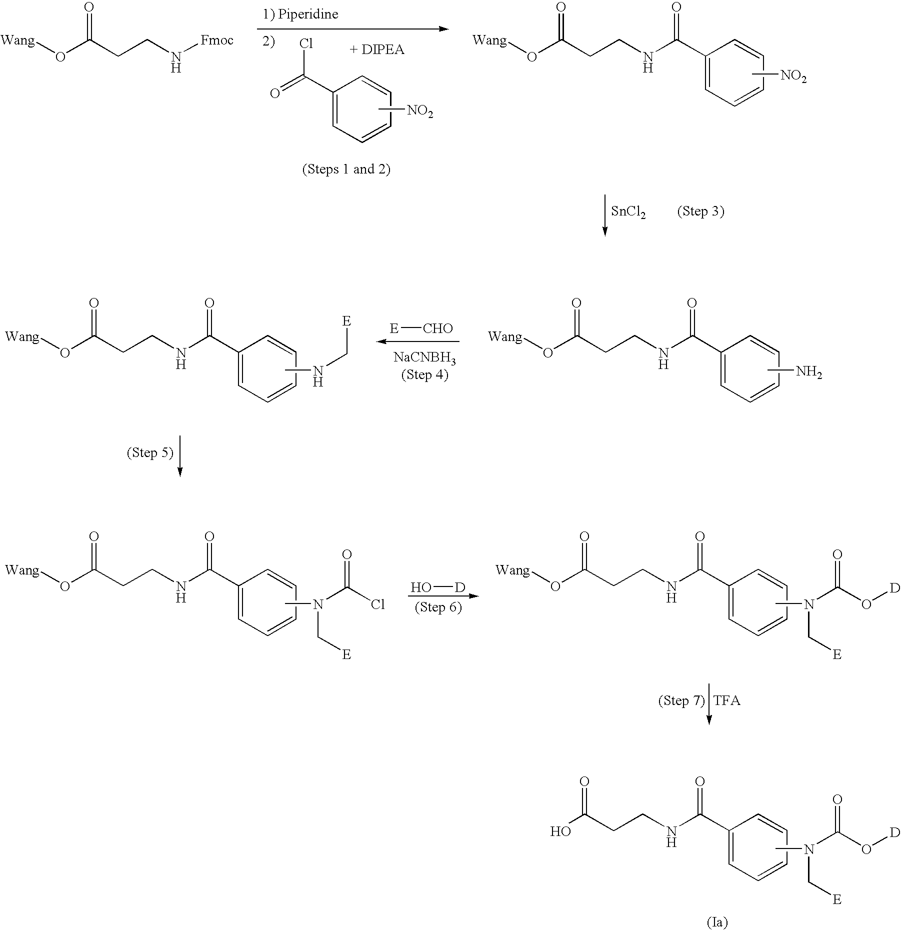

wherein E and D independently are aryl or heteroaryl and are both optionally substituted as defined above.

Steps 1 to 2:

These steps are analogous to the corresponding steps described in WO 00/69810 and WO 02/00612.

Step 3:

The reduction of aromatic nitro groups on solid phase is generally known (F. Z. Dörwald, “Organic Synthesis on Solid Phase”, 1^{st} Edition Wiley-VCH: Weinheim, 2000, p. 246-247), and is performed using an excess of tin (II) chloride dihydrate in a polar organic solvent such as DMF or NMP. The reaction is performed at 20-100° C., preferable at ambient or slightly elevated temperature.

Step 4:

This reaction is generally known (F. Z. Dörwald, “Organic Synthesis on Solid Phase”, 1^{st} Edition Wiley-VCH: Weinheim, 2000, p. 239-241), and is achieved by using an excess of aldehyde, sodium cyano borohydride and a proton source such as acetic acid. The reaction is performed at 20-100° C. preferable at 40-80° C. in a polar organic solvent such as DMF or NMP.

Steps 5:

The formation of carbamoyl chlorides from amines tethered on solid support is a know reaction (Wang, G. T. et al.; Tetrahedron Lett, 1997, 38(11), 1895-1898, Scicinski, J. J.; Barker, M. D.; Murray, P. J.; Jarvie, E. M.; Bioorg Med Chem Lett 1998, 8 (24), 3609-3614), and is generally performed by adding phosgene (or a synthetic equivalent such as bis(trichloromethoxy)carbonate or trichloromethoxycarbonyl chloride) to resin bound primary or secondary amine in the presence of base. As base, an organic amine such as triethylamine, pyridine, or DIPEA can be used. The reaction is preferably performed at 0-20° C., in an inert aprotic solvent such as DCM, toluene, DCP, THF or the like.

Step 6:

This reaction is known (Sabie, R. et al., Synth Commun 1990, 20(11), 1713-1719, Hansen, K. T.; Faarup, P.; Bundgaard, H.; J Pharm Sci, 1991, 80 (8), 793-798) and is performed by reacting carbamoyl chlorides with aryl- or hetero aryl alcohols in the presence of base. As base, an organic amine such as triethylamine, pyridine, DIPEA or 1,4-diazabicyclo[2.2.2.1octane (DABCO) can be used. The reaction is performed at 0-80° C., preferably at ambient temperature in an inert aprotic polar solvent such as DMF, THF or NMP.

Step 7: Cleavage from Resin

This step is analogous to the corresponding transformations described in WO 00/69810 and WO 02/00612.

The general procedure (A) is further illustrated in the following example:

### Example 1

### General Procedure (A)

3-{4-[(4-tert-Butylphenoxycarbonyl)-(9H-fluoren-2-ylmethyl)amino]benzoylamino}propionic acid

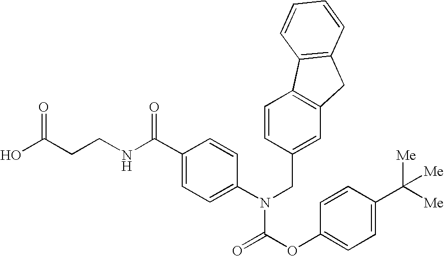

Step 1: Fmoc-β-Ala-Wang resin (5.0 g, 0.31 mmol/g, 1.55 mmol) was treated with piperidine (20% in NMP, 20 ml) for 30 min and the resin was drained. This was repeated once. The resin was then washed with DMF (5×).

Step 2: A solution of DIPEA (3 ml) in NMP (17 ml) was added, followed by slow addition of a solution of p-nitrobenzoylchloride (2.88 g; 15.5 mmol, 10 eq.) in NMP (20 ml). The mixture was shaken for 3 h, then drained. The resin was washed with DMF (5×).

Step 3: Then a solution of SnCl₂.2H₂O (10.5 g; 46.5 mmol, 30 eq.) in NMP (30 ml) was added. The mixture was shaken at room temperature for 16 h. The resin was drained and washed with DMF (3×) and DCM (10×), then dried under vacuum for 16 h to give 5.20 g of resin bound 4-aminobenzoylaminopropanoic acid.

Step 4: The dry resin (100 mg; 54 mmol; 0.54 mmol/g) prepared as described in step 3, was swelled in DCM for 30 min. Fluorene-2-carboxaldehyde (194.5 mg; 1 mmol) dissolved in DMF (1 ml) was added followed by a solution of sodium cyano borohydride (138 mg; 2 mmol) in DMF—acetic acid (1.2 ml, 5:1). The reaction mixture was heated to 80° C. over night. The resin was drained for solvent and reactants, and subsequently washed with MeOH (3×), DMF (5×) and DCM (4×) to give resin bound 3-{4-[(9H-fluoren-2-ylmethyl)amino]benzoylamino}propionic acid.

Step 5: Resin bound 3-{4-[(9H-fluoren-2-ylmethyl)amino]benzoylamino}propionic acid (100 mg; 0.54 mmol/g) was suspended in DCM (500 ul) and DIPEA (100 ul) was added. A solution of bis(trichloromethyl)carbonate (44 mg; 0.15 mmol) in DCM (500 ul) was then added. The reaction was shaken for 60 min. Then the solvent was drained and the resin was washed with DCM (4×), to give resin bound 3-{4-[chlorocarbonyl-(9H-fluoren-2-ylmethyl)amino]benzoylamino}propionic acid.

Step 6: To resin bound 3-{4-[chlorocarbonyl-(9H-fluoren-2-ylmethyl)amino]benzoylamino}propionic acid, was added a solution of 4-t-butylphenol (75 mg; 0.5 mmol) in DMF (500 ul) followed by a solution of DABCO (100 mg) in DMF (500 ul). The reaction was stirred over-night at ambient temperature, then washed with DMF (3×), 10% HOAc-MeOH (3×) and DCM (10×).

Step 7: The product was cleaved from the resin using 50% TFA in DCM. Solvent was removed by speed evacuation to give 10-12 mg of pure title material.

HPLC-MS (Method (B)): m/z: 563 (M+1), Rt: 5.20 min.

The following compounds were prepared in a similar manner

### Example 2

### General Procedure (A)

3-{4-[(Biphenyl-4-yloxycarbonyl)-(4-trifluoromethoxybenzyl)amino]benzoylamino}propionic acid

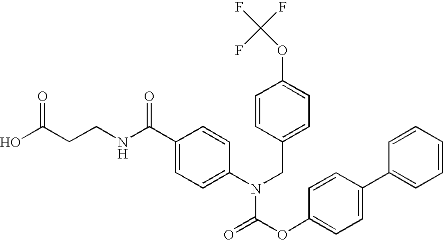

HPLC-MS-(Method (A)): m/z: 579 (M+1)

### Example 3

### General Procedure (A)

3-{4-[(Biphenyl-4-yloxycarbonyl)-(4-cyclohexylbenzyl)amino]benzoylamino}propionic acid

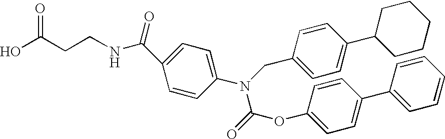

HPLC-MS-(Method (A)): m/z: 577 (M+1)

HPLC-MS-(Method (B)): m/z: 577 (M+1)

### Example 4

### General Procedure (A)

3-{4-[(Biphenyl-4-yloxycarbonyl)-(3-phenylallyl)amino]benzoylamino}propionic acid

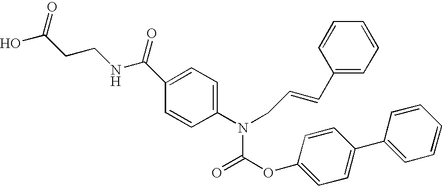

HPLC-MS-(Method (A)): m/z: 521 (M+1) Rt: 5.85 min

HPLC-MS-(Method (B)): m/z: 521 (M+1) Rt: 4.92 min

### Example 5

### General Procedure (A)

3-{4-[(Dibenzofuran-2-yloxycarbonyl)-(4-trifluoromethoxybenzyl)amino]benzoylamino}-propionic acid

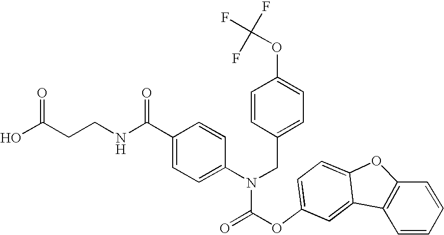

HPLC-MS-(Method (A)): m/z 593 (M+1) Rt: 7.17 min (1A)

HPLC-MS-(Method (B)): m/z 593 (M+1) Rt: 5.08 min (2A)

### Example 6

### General Procedure (A)

3-{4-[(4′-Cyano-biphenyl-4-yloxycarbonyl)-(4-trifluoromethoxybenzyl)amino]benzoylamino}-propionic acid

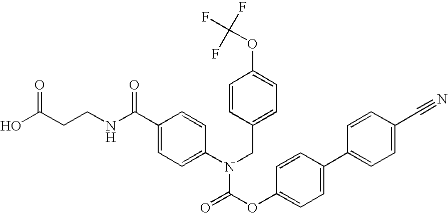

HPLC-MS-(Method (A)): m/z 604 (M+1) Rt: 6.93 min (1A)

### Example 7

### General Procedure (A)

3-{4-[(4-Cyclohexylphenoxycarbonyl)-(4-trifluoromethoxybenzyl)amino]benzoylamino}-propionic acid

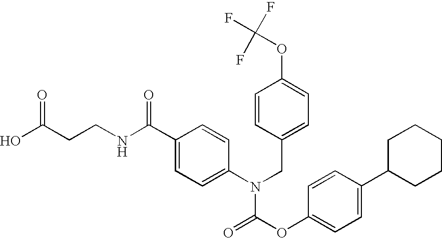

HPLC-MS-(Method (A)): m/z 585 (M+1) Rt: 7.83 min (1A)

HPLC-MS-(Method (B)): m/z 585 (M+1) Rt: 5.66 min (2A)

### Example 8

### General Procedure (A)

3-{4-[(Biphenyl-3-yloxycarbonyl)-(4-trifluoromethoxybenzyl)amino]benzoylamino}propionic acid

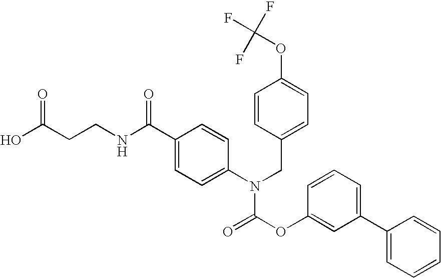

HPLC-MS-(Method (A)): m/z 579 (M+1) Rt: 7.17 min (1A)

HPLC-MS-(Method (B)): m/z 579 (M+1) Rt: 5.07 min (2A)

### Example 9

### General Procedure (A)

3-{4-[(4′-Bromobiphenyl-4-yloxycarbonyl)-(4-trifluoromethoxybenzyl)amino]benzoylamino}-propionic acid

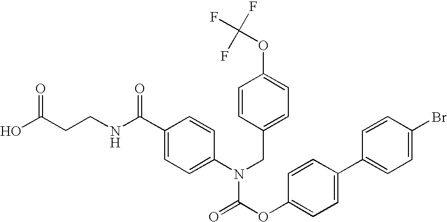

HPLC-MS-(Method (A)): m/z 658 (M+1) Rt: 7.63 min (1A)

### Example 10

### General Procedure (A)

3-{4-[(Dibenzofuran-2-yloxycarbonyl)-(3-trifluoromethoxybenzyl)amino]benzoylamino}-propionic acid

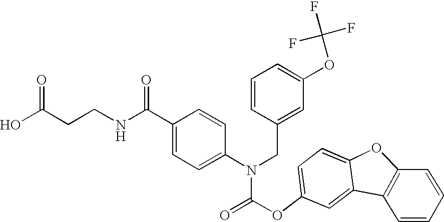

HPLC-MS-(Method (A)): m/z 593 (M+1) Rt: 7.10 min (1A)

### Example 11

### General Procedure (A)

3-{4-[Biphenyl-4-ylmethyl-(biphenyl-4-yloxycarbonyl)amino]benzoylamino}propionic acid

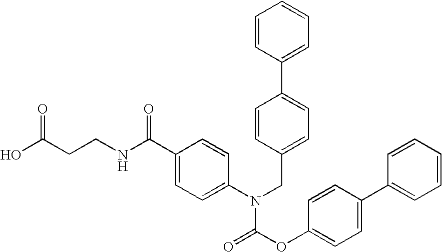

HPLC-MS (Method (B)): m/z: 571 (M+1), Rt: 5.33 min.

### Example 12

### General Procedure (A)

3-{4-[Biphenyl-4-ylmethyl-(4-trifluoromethoxyphenoxycarbonyl)amino]benzoylamino}-propionic acid

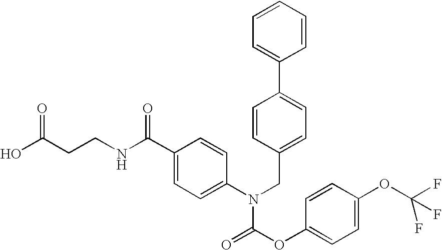

HPLC-MS (Method (B)): m/z: 579 (M+1), Rt: 5.16 min

### Example 13

### General procedure (A)

3-{4-[Biphenyl-4-ylmethyl-(3-trifluoromethoxyphenoxycarbonyl)amino]benzoylamino}-propionic acid

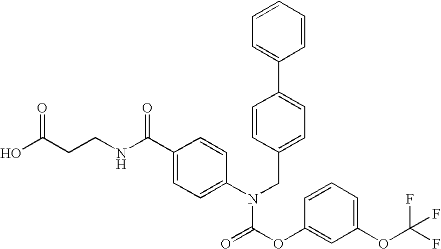

HPLC-MS (Method (B)): m/z: 579 (M+1), Rt: 5.15 min

### Example 14

### General Procedure (A)

3-{4-[(Biphenyl-4-yloxycarbonyl)-(4-cyclohexylbenzyl)amino]benzoylamino}propionic acid

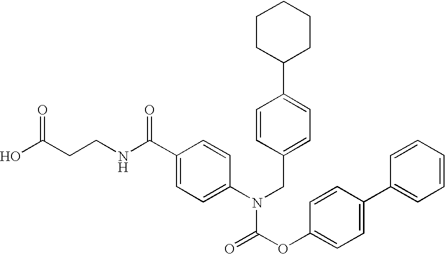

HPLC-MS (Method (B)): m/z: 577 (M+1), Rt: 4.78 min

### Example 15

### General Procedure (A)

3-{4-[(Biphenyl-4-yloxycarbonyl)-(4-cyclohexylbenzyl)amino]benzoylamino}propionic acid

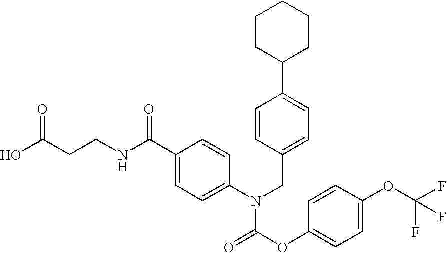

HPLC-MS (Method (B)): m/z: 585 (M+1), Rt: 5.72 min

### Example 16

### General Procedure (A)

3-{4-[(4-Cyclohexylbenzyl)-(3-trifluoromethoxyphenoxycarbonyl)amino]benzoylamino}-propionic acid

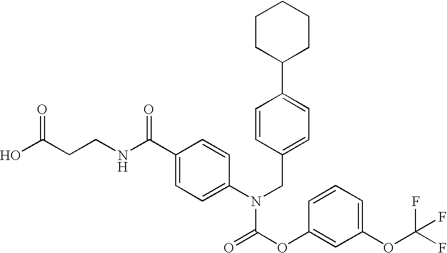

HPLC-MS (Method (B)): m/z: 585 (M+1), Rt: 5.71 min

### Example 17

### General Procedure (A)

3-{4-[(Biphenyl-4-yloxycarbonyl)-(4-tert-butylbenzyl)amino]benzoylamino}propionic acid

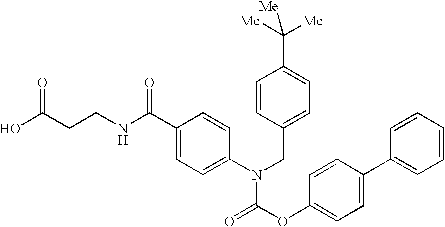

HPLC-MS (Method (B)): m/z: 551 (M+1), Rt: 5.47 min

### Example 18

### General Procedure (A)

3-{4-[(4-tert-Butylbenzyl)-(4-trifluoromethoxyphenoxycarbonyl)amino]benzoylamino}-propionic acid

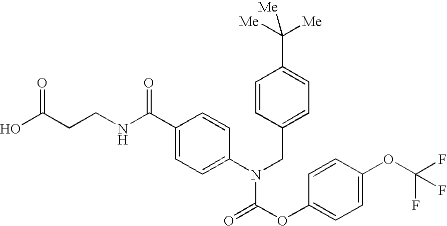

HPLC-MS (Method (B)): m/z: 559 (M+1), Rt: 5.30 min

### Example 19

### General Procedure (A)

3-{4-[(4-tert-Butylbenzyl)-(3-trifluoromethoxyphenoxycarbonyl)amino]benzoylamino}-

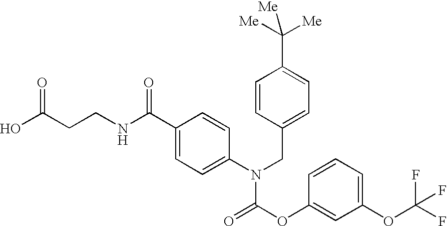

HPLC-MS (Method (B)): m/z: 559 (M+1), Rt: 5.30 min

### Example 20

### General Procedure (A)

3-{4-[(Biphenyl-4-yloxycarbonyl)-(4-phenoxybenzyl)amino]benzoylamino}propionic acid

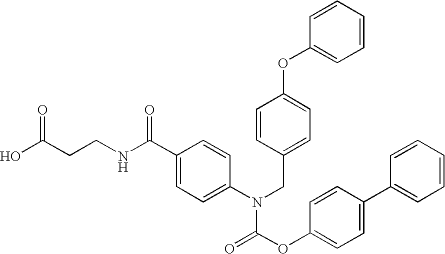

HPLC-MS (Method (B)): m/z: 587 (M+1), Rt: 5.33 min

### Example 21

### General Procedure (A)

3-{4-[(4-Phenoxybenzyl)-(3-trifluoromethoxyphenoxycarbonyl)amino]benzoylamino}propionic acid

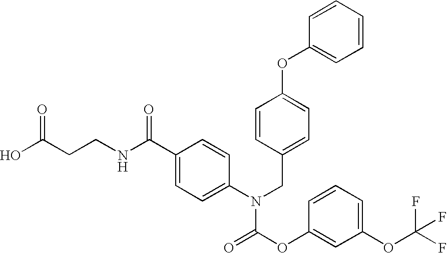

HPLC-MS (Method (B)): m/z: 595 (M+1), Rt: 5.05 min

### Example 22

### General Procedure (A)

3-{4-[(4-Trifluoromethoxybenzyl)-(4-trifluoromethoxyphenoxycarbonyl)amino]benzoylamino}-propionic acid

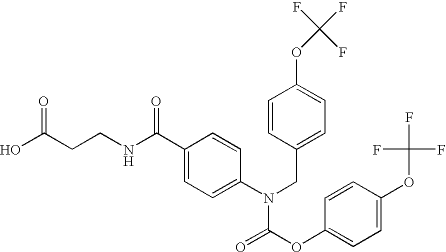

HPLC-MS (Method (B)): m/z: 587 (M+1), Rt: 4.94 min

### Example 23

### General procedure (A)

3-{4-[(4′-Cyano-biphenyl-4-yloxycarbonyl)-(3-trifluoromethoxybenzyl)amino]benzoylamino}-propionic acid

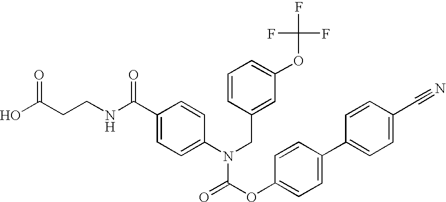

HPLC-MS (Method (B)): m/z: 604 (M+1), Rt: 4.94 min

### Example 24 General Procedure (A)

3-{4-[(4-Cyclohexylphenoxycarbonyl)-(3-trifluoromethoxybenzyl)amino]benzoylamino}-propionic acid

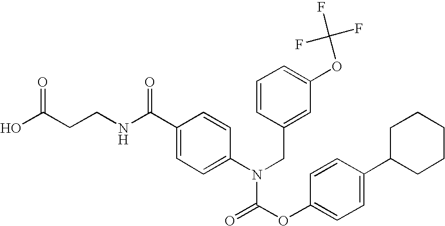

HPLC-MS (Method (B)): m/z: 585 (M+1), Rt: 7.75 min

### Example 25

### General Procedure (A)

3-{4-[(Biphenyl-3-yloxycarbonyl)-(3-trifluoromethoxybenzyl)amino]benzoylamino}propionic acid

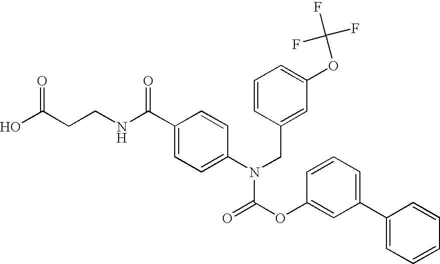

HPLC-MS (Method (B)): m/z: 579 (M+1), Rt: 7.12 min

### Example 26 General Procedure (A)

3-{4-[(Biphenyl-4-yloxycarbonyl)-(3-trifluoromethoxybenzyl)amino]benzoylamino}propionic acid

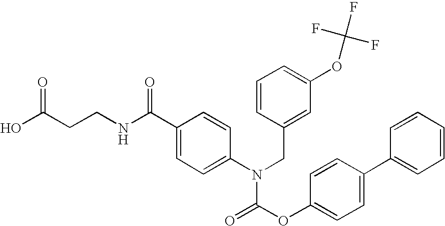

HPLC-MS (Method (B)): m/z: 579 (M+1), Rt: 7.09 min

### Example 27

### General Procedure (A)

3-{4-[(4′-Bromobiphenyl-4-yloxycarbonyl)-(3-trifluoromethoxybenzyl)amino] benzoylamino}propionic acid

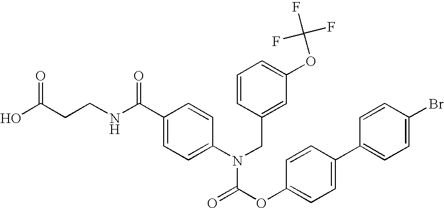

HPLC-MS (Method (B)): m/z: 659 (M+1), Rt: 7.57 min

### Example 28

### General Procedure (A)

3-{4-[(4-Cyclohexylbenzyl)-(2-methylbenzothiazol-5-yloxycarbonyl)amino]benzoylamino}-propionic acid

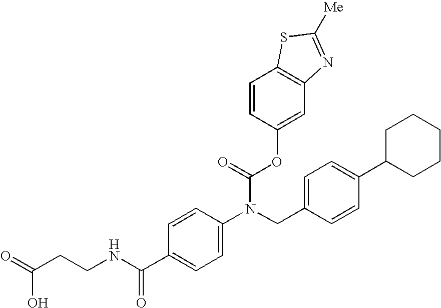

### Example 29

### General Procedure (A)

3-{4-[(Biphenyl-3-yloxycarbonyl)-(4-cyclohexylbenzyl)amino]benzoylamino}propionic acid

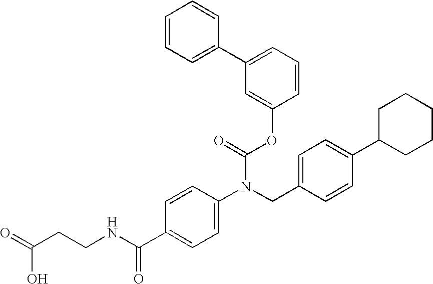

### Example 30

### General Procedure (A)

3-{4-[(4-Isopropyl-3-methylphenoxycarbonyl)-(4-trifluoromethoxybenzyl)amino]benzoylamino}propionic acid

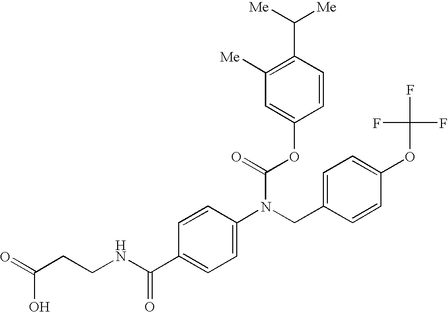

### Example 31

### General Procedure (A)

3-{4-[(3,5-Dichlorophenoxycarbonyl)-(4-trifluoromethoxybenzyl)amino]benzoylamino}-propionic acid

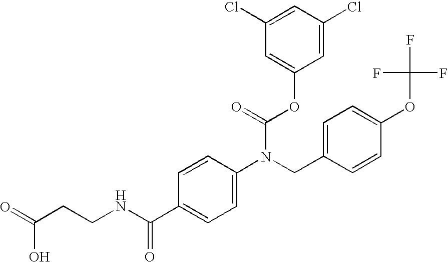

### Example 32

### General Procedure (A)

3-{4-[(4-Trifluoromethoxybenzyl)-(3-trifluoromethoxyphenoxycarbonyl)amino]benzoylamino}-propionic acid

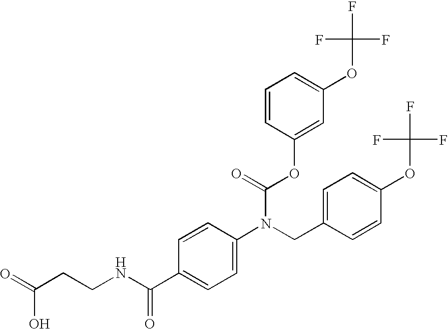

### Example 33

### General Procedure (A)

3-{4-[(4-Cyclohexylphenoxycarbonyl)-(4-trifluoromethoxybenzyl)amino]benzoylamino}-propionic acid

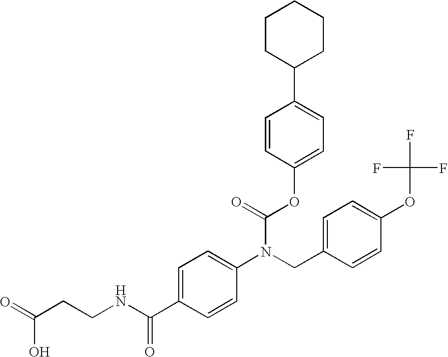

### Example 34

### General Procedure (A)

3-{4-[Biphenyl-4-ylmethyl-(3,5-dichlorophenoxycarbonyl)amino]benzoylamino}propionic acid

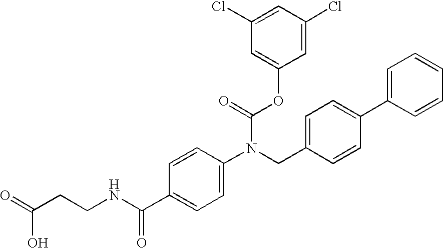

### Example 35

### General Procedure (A)

3-{4-[(4-Bromobenzyl)-(dibenzofuran-2-yloxycarbonyl)amino]benzoylamino}propionic acid

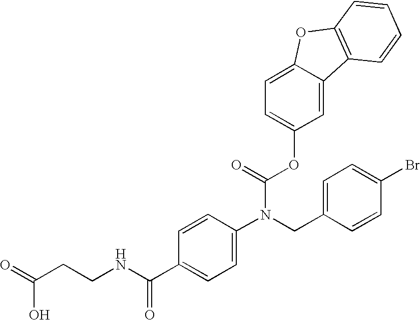

### Example 36

### General Procedure (A)

3-{4-[(Biphenyl-3-yloxycarbonyl)-(4-bromobenzyl)amino]benzoylamino}propionic acid

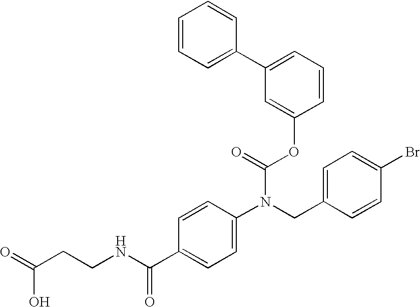

### Example 37

### General Procedure (A)

3-{4-[(4-Bromobenzyl)-(4′-bromobiphenyl-4-yloxycarbonyl)amino]benzoylamino}propionic acid

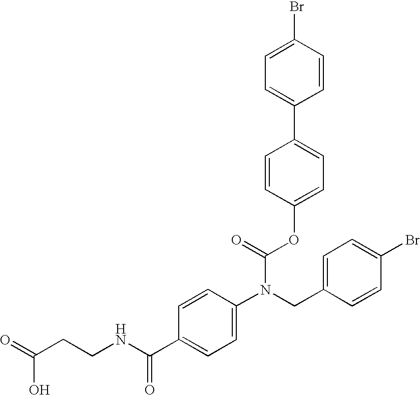

### Example 38

### General Procedure (A)

3-{4-[(4-Bromobenzyl)-(3,5-dichlorophenoxycarbonyl)amino]benzoylamino}propionic acid

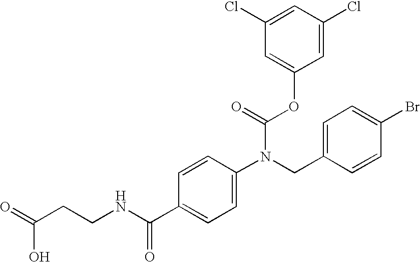

### Example 39

### General Procedure (A)

3-{4-[(4-Bromobenzyl)-(4-trifluoromethoxyphenoxycarbonyl)amino]benzoylamino}propionic acid

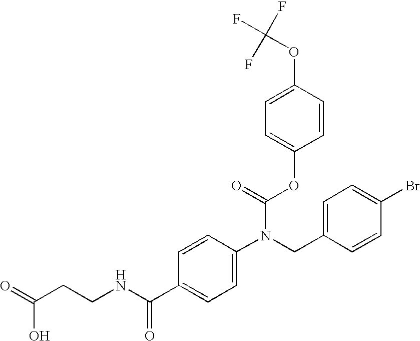

### Example 40

### General Procedure (A)

3-{4-(9H-Fluoren-2-ylmethyl)-(2-methylbenzothiazol-5-yloxycarbonyl)amino]benzoylamino}propionic acid

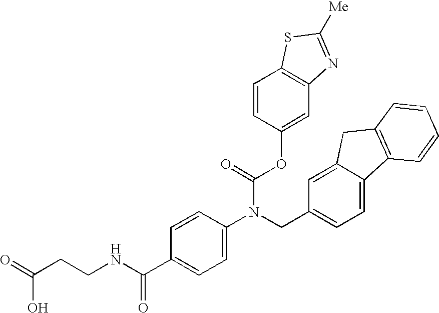

### Example 41

### General Procedure (A)

3-{4-[(4-tert-Butylphenoxycarbonyl)-(9H-fluoren-2-ylmethyl)amino]benzoylamino}propionic acid

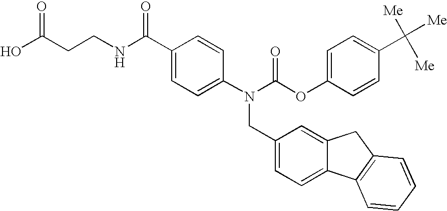

HPLC-MS (Method (B)): m/z: 563 (M+1), Rt: 5.20 min.

### Example 42

### General Procedure (A)

3-{4-[(4-Cyclohexylphenoxycarbonyl)-(9H-fluoren-2-ylmethyl)amino]benzoylamino}propionic acid

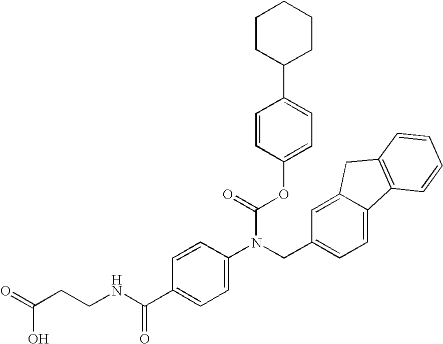

HPLC-MS (Method (B)): m/z: 589 (M+1), Rt: 5.61 min.

### Example 43

### General Procedure (A)

3-{4-[(4′-Bromobiphenyl-4-yloxyarbonyl)-(4-tert-butylbenzyl)amino]benzoylamino}propionic acid

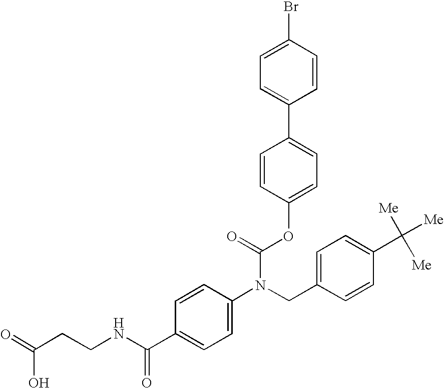

### Example 44

### General Procedure (A)

3-{4-[(3,5-Dichlorobenzyl)-(3,5-dichlorophenoxycarbonyl)amino]benzoylamino}propionic acid

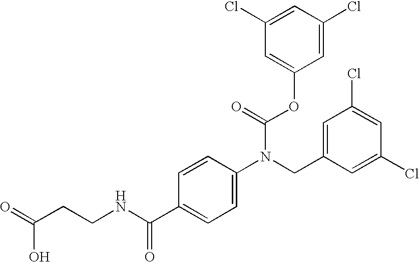

HPLC-MS (Method (B)): m/z: 557 (M+1), Rt: 5.46 min.

### Example 45

### General Procedure (A)

3-{4-[(3-Trifluoromethoxybenzyl)-(4-trifluoromethoxyphenoxycarbonyl)amino]benzoylamino}-propionic acid

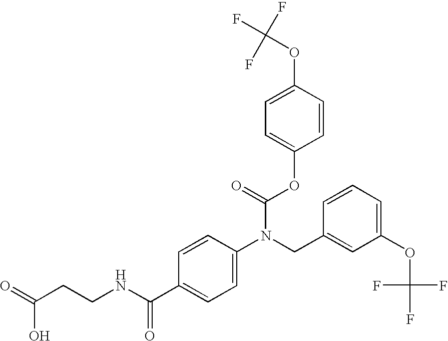

### Example 46

### General Procedure (A)

3-{4-[(3,5-Bis(trifluoromethyl)benzyl)-(dibenzofuran-2-yloxycarbonyl)amino]benzoylamino}-propionic acid

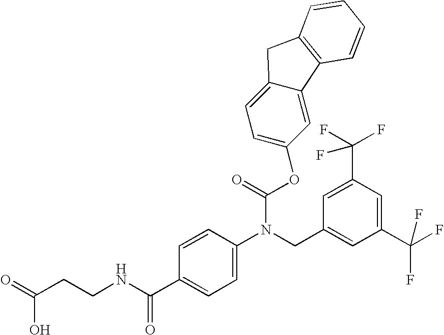

### Example 47

### General Procedure (A)

3-{4-[(Biphenyl-4-yloxycarbonyl)-(3,5-bis(trifluoromethyl)benzyl)amino]benzoylamino}-propionic acid

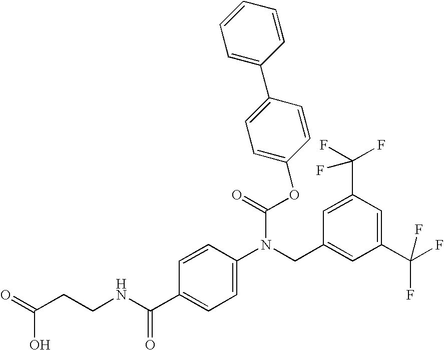

### Example 48

### General Procedure (A)

3-{4-[(3,5-Bis{trifluoromethyl}benzyl)-(3-trifluoromethoxyphenoxycarbonyl)amino]benzoylamino}propanoic acid

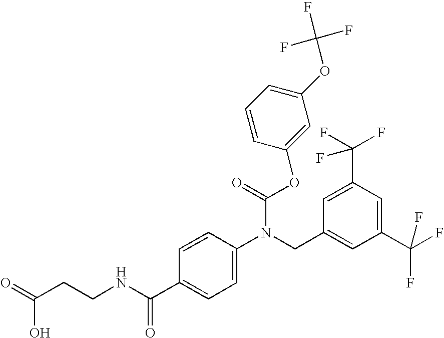

### Example 49

### General Procedure (A)

3-{3-[(3,5-Bis(trifluoromethyl)benzyl)-(4′-cyanobiphenyl-4-yloxycarbonyl)amino]benzoylamino}propionic acid

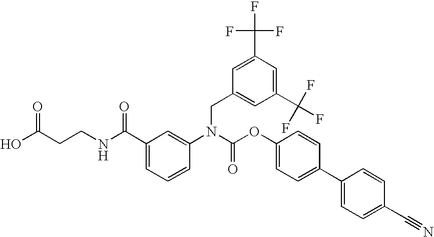

HPLC-MS-(Method (A)): m/z: 656 (M+1) Rt: 6.95 min

### Example 50

### General Procedure (A)

3-{3-[(3,5-Bis(trifluoromethyl)benzyl)-(dibenzofuran-2-yloxycarbonyl)amino]benzoylamino}-propionic acid

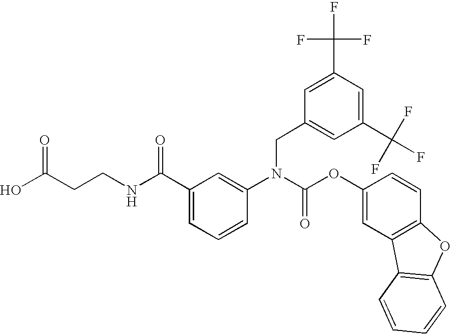

HPLC-MS-(Method (A)): m/z 645 (M+1) Rt: 7.19 min

### Example 51

### General Procedure (A)

3-(3{(4′-Cyanobiphenyl-4-yloxycarbonyl)-[3-(3,4-dichlorophenoxy)benzyl]amino}benzoylamino)propionic acid

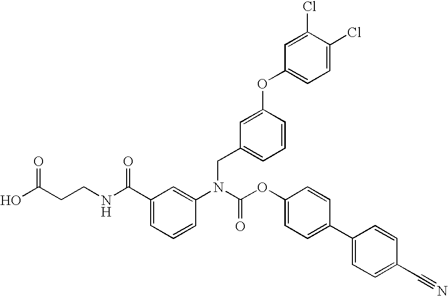

HPLC-MS-(Method (A)): m/z 681 (M+1) Rt: 7.47 min

### Example 52

### General Procedure (A)

3-{3-[(Dibenzofuran-2-yloxycarbonyl)-(4-trifluoromethoxybenzyl)amino]benzoylamino}-propionic acid

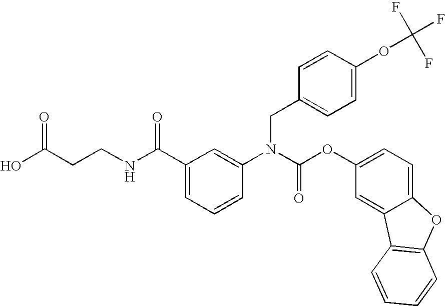

HPLC-MS-(Method (A)): m/z 593 (M+1) Rt: 7.09 min

### Example 53

### General Procedure (A)

3-{3-[(4′-Cyanobiphenyl-4-yloxycarbonyl)-(4-trifluoromethoxybenzyl)amino]benzoylamino}-propionic acid

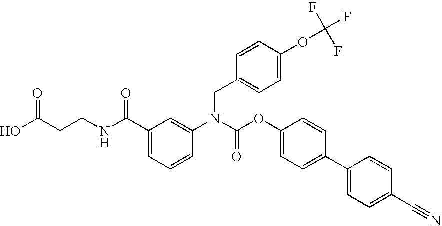

HPLC-MS-(Method (A)): m/z 604 (M+1) Rt: 6.90 min

### Example 54

### General Procedure (A)

3-{3-[(4-Cyclohexylphenoxycarbonyl)-(4-trifluoromethoxybenzyl)amino]benzoylamino}-propionic acid

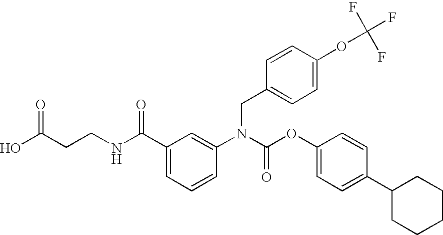

HPLC-MS-(Method (A)): m/z 585 (M+1) Rt: 7.80 min

### Example 55

### General Procedure (A)

3-{3-[(Biphenyl-3-yloxycarbonyl)-(4-trifluoromethoxybenzyl)amino]benzoylamino}propionic acid

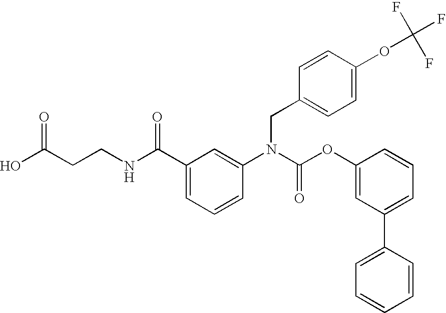

HPLC-MS-(Method (A)): m/z 579 (M+1) Rt: 7.10 min

### Example 56

### General Procedure (A)

3-{3-[(Biphenyl-4-yloxycarbonyl)-(4-trifluoromethoxybenzyl)amino]benzoylamino}propionic acid

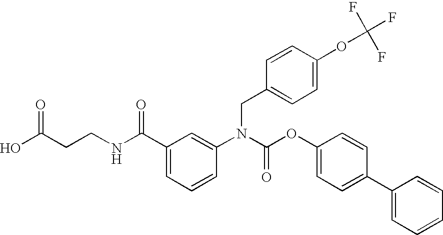

HPLC-MS-(Method (A)): m/z 579 (M+1) Rt: 7.20 min

### Example 57

### General Procedure (A)

3-{3-[(4′-Bromobiphenyl-4-yloxycarbonyl)-(4-trifluoromethoxybenzyl)amino]benzoylamino}-propionic acid

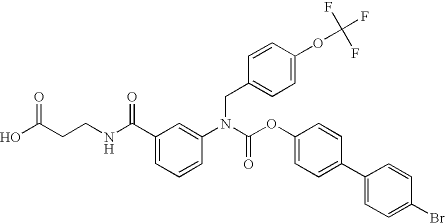

HPLC-MS-(Method (A)): m/z 658 (M+1) Rt:7.67 min

### Example 58

### General Procedure (A)

3-{3-[(Dibenzofuran-2-yloxycarbonyl)-(3-trifluoromethoxybenzyl)amino]benzoylamino}-propionic acid

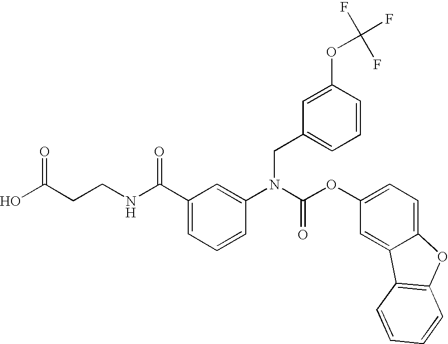

HPLC-MS-(Method (A)): m/z 593 (M+1) Rt:7.13 min

### Example 59

### General Procedure (A)

3-{3-[(4′-Cyanobiphenyl-4-yloxycarbonyl)-(3-trifluoromethoxybenzyl)amino]benzoylamino}propionic acid

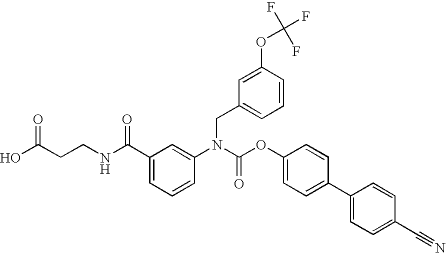

HPLC-MS-(Method (A)): m/z 604 (M+1) Rt: 6.87 min

### Example 60

### General Procedure (A)

3-{3-[(4-Cyclohexylphenoxycarbonyl)-(3-trifluoromethoxybenzyl)amino]benzoylamino}-propionic acid

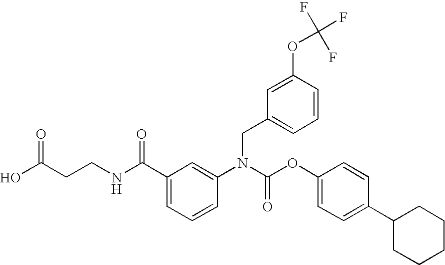

HPLC-MS-(Method (A)): m/z 585 (M+1) Rt: 7.77 min

### Example 61

### General Procedure (A)

3-{3-[(Biphenyl-3-yloxycarbonyl)-(3-trifluoromethoxybenzyl)amino]benzoylamino}propionic acid

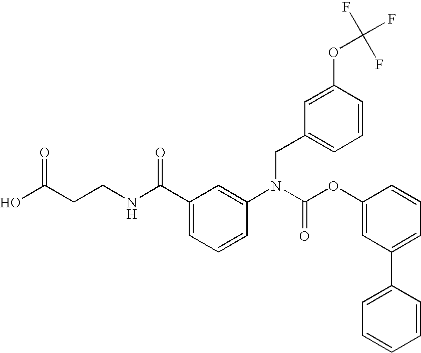

HPLC-MS-(Method (A)): m/z 579 (M+1) Rt: 7.10 min

### Example 62

### General Procedure (A)

3-3-[(Biphenyl-4-yloxycarbonyl)-(3-trifluoromethoxybenzyl)amino]benzoylamino}propionic acid

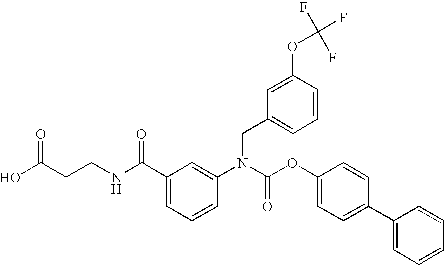

HPLC-MS-(Method (A)): m/z 579 (M+1) Rt: 7.17 min

### Example 63

### General Procedure (A)

3-{3-[(4′-Bromobiphenyl-4-yloxycarbonyl)-(3-trifluoromethoxybenzyl)amino]benzoylamino}-propionic acid

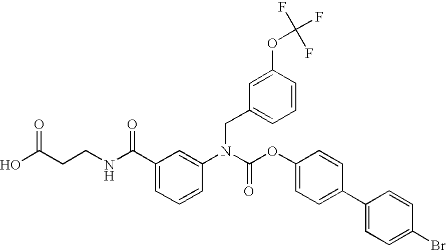

HPLC-MS-(Method (A)): m/z 658 (M+1) Rt: 7.60 min

### Example 64

### General Procedure (A)

3-{3-[(Biphenyl-4-yloxycarbonyl)-(4-cyclohexylbenzyl)amino]benzoylamino}propionic acid

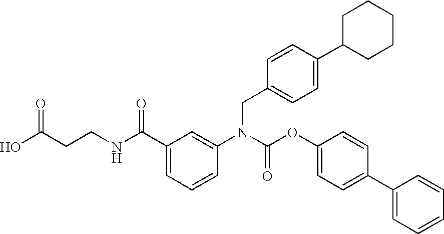

General Procedure B:

General procedure (B) may be used for solution phase preparation of compounds of general formula (Ia):

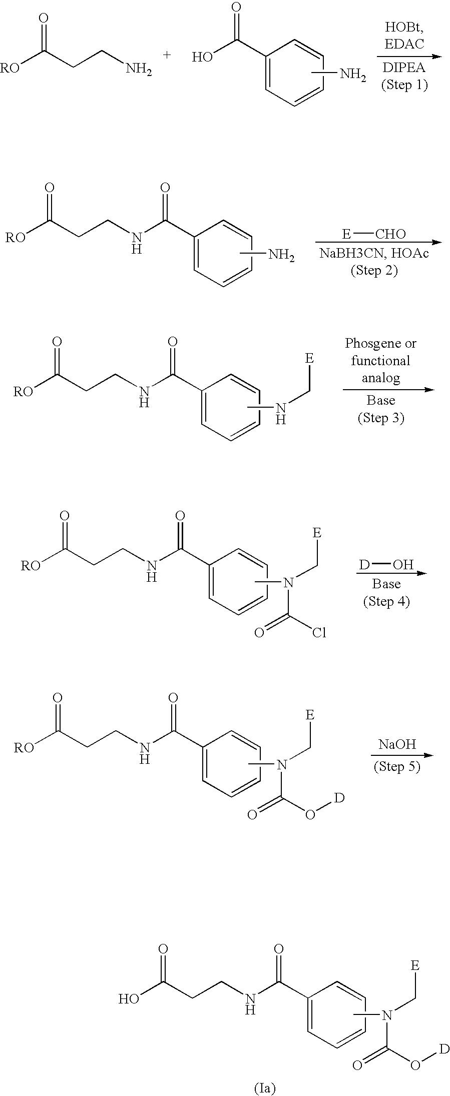

wherein R are C₁₋₆-alkyl, and E and D independently are aryl or heteroaryl and are both optionally substituted as defined above.

Step 1:

This is a coupling reaction between an amino benzoic acid and an β-alanine ester. The step is similar to transformations described in WO 00/69810

Step 2:

The reductive amination steps are analogous to the corresponding steps described in WO 00/69810.

Steps 3:

The formation of carbamoyl chlorides from amines is a known reaction (Heusler, K.; Helv. Chim. Acta, 1972, 55, 388), and is generally performed by adding phosgene (or a synthetic equivalent such as bis(trichloromethoxy)carbonate or trichloromethoxycarbonyl chloride) to a secondary amine in the presence of base. As base, an organic amine such as triethylamine, pyridine, or DIPEA can be used. The reaction is preferably performed at 0-20° C., in an inert aprotic solvent such as DCM, toluene, DCP, THF or the like.

Step 4:

This reaction is known (Wuest, H. M.; Sakal, E. H.; J Am Chem Soc, 1951, 73, 1210.) and is performed by reacting carbamoyl chlorides with aryl- or hetero aryl alcohols in the presence of base. As base, an organic amine such as triethylamine, pyridine, DIPEA or 1,4-diazabicyclo[2.2.2.]octane (DABCO) can be used. The reaction is performed at 0-80° C., preferably at ambient temperature in an inert aprotic polar solvent such as DMF, THF or NMP.

Step 5:

Steps 5 is an ester hydrolysis, and is performed analogue to similar transformations described in WO 00/69810.

The general procedure (B) is further illustrated in the following example:

### Example 65

### General Procedure (B)

3-{4-[(4-tert-Butylphenoxycarbonyl)-(9H-fluoren-2-ylmethyl)amino]benzoylamino}propionic acid

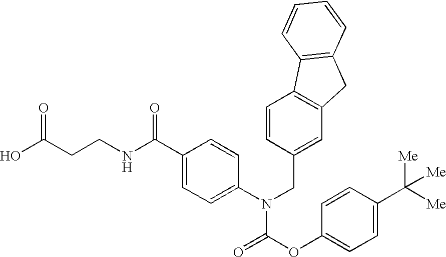

3-(4-Aminobenzoylamino)propionic acid ethyl ester

4-Aminobenzoic acid (4.50 g; 32.5 mmol) and 1-hydroxybenzotriazole hydrate (5.50 g; 36.0 mmol) was dissolved in THF (100 ml). 1-Ethyl-3-(3′-dimethylaminopropyl)carbodiimide hydrochloride (6.80 g; 36 mmol) was added. A semi crystalline solid appeared. Dichloromethane (50 ml) was added and a clear solution was obtained. The mixture was stirred for an additional 10 min.; then ethyl 3-aminopropanoic acid hydrochloride (5.00 g; 32.5 mmol) and diisopropylethylamine (17.0 ml; 97.5 mmol) were added. The mixture was stirred for 2 h at ambient temperature, and then heated to reflux for 10 min. The reaction mixture was allowed to cool, then partitioned between water (100 ml) and DCM (100 ml). The water phase was extracted trice with DCM (3×100 ml). The combined organic phases were then dried over anhydrous sodium sulphate, filtered and evaporated to dryness by rotary evaporation in vacuo. A quantitative yield of an amber coloured oil was obtained, which crystallized upon standing. HPLC-MS (Method (B)): m/z: 237 (M+1), Rt: 1.44 min. ¹H-NMR(DMSO-d₆): δ 1.15 ppm (t, 3H); 2.50 (t,2H, collapse with the DMSO-d₅ signal); 3.55 (q, 2H); 4.02 (q, 2H); 6.51 (d, 2H); 7.53 (d, 2H); 8.04 (t, 1H).

3-{4[(9H-Fluoren-2-ylmethyl)amino]benzoylamino}propionic acid ethyl ester

3-(4-Aminobenzoylamino)propionic acid ethyl ester (12.0 g; 50.8 mmol) was dissolved in ethanol (200 ml). Then a solution of fluorene-2-carboxaldehyde (9.86 g; 50.8 mmol) in ethanol (100 ml) was added to give a clear yellow solution. The solution was heated for reflux for 10 min. then cooled to room temperature. Acetic acid (30 ml) and solid sodium cyano borohydride (3.5 g; 50.7 mmol) were added. The mixture was refluxed for 30 min; then slowly allowed to cool to room temperature. The title material, which slowly crystallised out of solution, was collected and washed with water. The crystals were then dried in an oven over night. Yield: 17.46 g (83%). HPLC-MS (Method (B)): m/z: 415 (M+1), Rt: 4.37 min. ¹H-NMR(CDCl₃): δ 1.25 ppm (t, 3H); 2.60 (t, 2H); 3.69 (q, 2H); 3.88 (s, 2H); 4.15 (q, 2H); 4.44 (s, 2H); 6.62 (d, 2H); 7.28-7.40 (m, 3H); 7.54 (d, 2H); 7.62 (d, 2H); 7.76 (t, 2H).

3-{4[(4-tert-Butyl phenoxycarbonyl)-(9H-fluoren-2-ylmethyl)amino]benzoylamino}propionic acid ethyl ester

3-{4-[(9H-Fluoren-2-ylmethyl)amino]benzoylamino}propionic acid ethyl ester (8.0 g; 19.3 mmol) was suspended in DCM (100 ml). Diisopropylethylamine (10.2 ml, 57.9 mmol) and a 20% solution of phosgene in toluene (20.0 ml; 38.7 mmol) was then carefully added, whereupon the reaction mixture turned clear. The mixture was stirred for 30 min, then partitioned between saturated aqueous sodium hydrogen carbonate (200 ml) and DCM (200 ml). The organic phase was collected and washed once with saturated aqueous sodium hydrogen carbonate (200 ml) and once with brine (250 ml), then dried over anhydrous sodium sulphate. The solvent was then removed by rotary evaporation to give the crude chlorocarbonyl derivatized 3-{4-[(9H-fluoren-2-ylmethyl)amino]benzoylamino}propionic acid ethyl ester as clear oil. The oil (9.2 g; 19.3 mmol) was re-dissolved in DCM (150 ml); then DABCO (2.16 g; 19.3 mmol) and 4-tert-butylphenol (2.90 g; 19.3 mmol) were added. The mixture was stirred at ambient temperature for 16 h, then partitioned between water (200 ml) and DCM (200 ml). The organic phase was washed with 0.2 N aqueous sodium hydroxide solution (5×250 ml) and once with brine (500 ml), then treated with anhydrous sodium sulphate and taken to dryness. The residual oil was dissolved in acetonitrile (300 ml) and re-evaporated to dryness to remove the remaining solvent impurities. Yield: 10.4 g (90%). HPLC-MS (Method (B)): m/z: 591 (M+1), Rt: 5.70 min. ¹H-NMR(DMSO-d₆): δ 1.15 ppm (t, 3H); 1.28 (s, 9H); 2.55 (t, 2H); 3.43 (m, 2H); 3.88 (s, 2H); 4.03 (q, 2H); 5.08 (s, 2H); 7.08 (d, 2H); 7.25-7.58 (m, 7H); 78 (d, 2H); 7.82 (d, 2H); 8.52 (t, 2H).

3-{4[(4-tert-Butylphenoxycarbonyl)-(9H-fluoren-2-ylmethyl)amino]benzoylamino}propionic acid

3-{4-[(4-tert-Butylphenoxycarbonyl)-(9H-fluoren-2-ylmethyl)amino]benzoylamino}propionic acid ethyl ester was dissolved in ethanol (100 ml) and the clear solution was cooled on an ice bath. 4N aqueous sodium hydroxide solution (100 ml) was slowly added. The solution, which remained clear was then stirred at room temperature for 1 h. Then acetic acid (40 ml) was added. The reaction was diluted with water (250 ml), to give a milky suspension, which was extracted with ethyl acetate (300 ml). The organic phase was separated, washed once with brine, dried with sodium sulphate and taken to dryness. The oily residue was stripped twice from acetonitril and once from dichloromethane, to give a clear faintly coloured glass. The glass was dissolved in toluene (50 ml). Petrolether (450 ml) was added to give a slow precipitation of white sticky material. The mixture was stirred at ambient temperature for 2 days, whereby a white solid was formed. The solid was filtered off, washed with cold petrol ether, and dried in a vacuum oven. Yield: 9.18 g (93%) of the title product.

LC-MS (Method (B)): m/z: 563 (M+1), Rt: 5.20 min. ¹H-NMR(CDCl₃): δ 2.66 (t, 2H); 3.68 (q, 2H); 3.86 (s, 2H); 5.05 (s, 2H); 6.75 (t, 1H); 7.00 (d, 2H); 7.25-7.38 (m, 7); 7.48 (s, 1H); 7.52 (d, 1H); 7.68 (d, 2H); 7.75 (d, 2H).

### Example 66

### General Procedure (B)

3-{4-[(4-Cyclohexylphenoxycarbonyl)-(9H-fluoren-2-ylmethyl)amino]benzoylamino}propionic acid

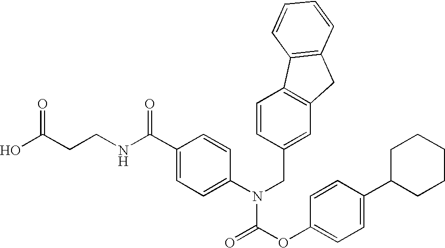

HPLC-MS (Method (B)): m/z: 589 (M+1), Rt: 5.61 min. ¹H-NMR(CDCl₃): δ 1.15-1.45 (m, 6H); 1.15-1.90 (m, 4H); 2.45 (m, 1H); 2.65 (t, 2H); 3.68 (q, 2H); 3.84 (s, 2H); 5.03 (s, 2H); 6.75 (t, 1H); 6.95 (d, 2H); 7.14 (d, 2H); 7.18-7.39 (m, 6H); 7.48 (s, 1H); 7.52 (d, 1H); 7.66 (d, 2H); 7.74 (d, 2H).

General Procedure (C)

General procedure (C) for solid phase synthesis of compounds of the general formula (I_{b}):

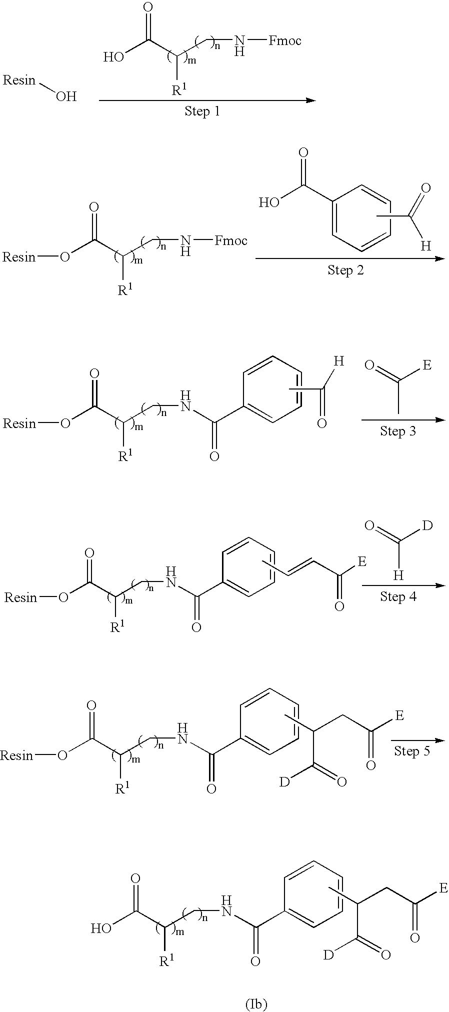

wherein D, E, m, n and R¹ are as defined for formula (I), and Resin is a polystyrene resin loaded with a Wang-linker.

Step 1:

This reaction is known (Wang S. J., J. Am. Chem. Soc. 95, 1328, 1973) and is generally performed by stirring polystyrene resin loaded with a linker such as the Wang linker with a 4-10 molar excess of Fmoc-protected amino acid activated with a 2-5 molar excess of diisopropylcarbodiimide, dicyclohexylcarbodiimide or 1-[3-(dimethylamino)propyl]-3-ethylcarbodiimide hydrochloride in the presence of a catalyst such as DMAP. The esterification is carried out in solvent such as THF, dioxane, toluene, DCM, DMF, NMP or a mixture of two or more of these. The reactions are performed between 0° C. and 80° C., preferably between 20° C. to 40° C. When the esterification is complete excess of reagent is removed by filtration. The resin is successively washed with the solvent used in the reaction, followed by washing with methanol. The resin bound product can be further dried and analyzed.

Step 2:

N-Fluorenylmethylcarbonyl protecting group is removed by treating the resin bound derivative with a 20%-50% solution of a secondary amine such as piperidine in a polar solvent such as DMF or NMP (Carpino L., Han G., J. Org. Chem. 37, 3404, 1972). The reaction is performed between 20° C. to 180° C., preferably between 20° C. to 40° C. When the reaction is complete excess of reagent is removed by filtration. The resin is successively washed with solvent used in the reaction. The resulting resin bound intermediate is acylated with acid. The acylation is known (The combinatorial index, Ed. Bunin B. A., 1998, Acedemic press, p. 78) and is generally performed by stirring the resin bound intermediate with a 2-5 molar excess of acid activated with a 2-5 molar excess of of diisopropyl-carbodiimide, dicyclohexylcarbodiimide or 1-[3-(dimethylamino)propyl]-3-ethylcarbodiimide hydrochloride in the presence of a side reaction inhibitor such as N-hydroxybenzotriazole. The acylation is carried out in a solvent such as THF, dioxane, toluene, DCM, DMF, NMP or a mixture of two or more of these. The reactions are performed between 0° C. to 80° C., preferably between 20° C. to 40° C. When the esterification is complete excess of reagent is removed by filtration. The resin is successively washed with the solvent used in the reaction, followed by washing with methanol. The resin bound product can be further dried and analyzed.

Step 3:

This reaction is a modification of previously described procedures for aldol condensation on solid support (U. Sensfuss, Tetrahedron Lett. 44, 2371-2374 (2003)). The reaction is carried out by reacting polystyrene-linked benzaidehydes with methyl ketones in presence of cobalt(II) or zinc acetate 2,2′-bipyridine complexes and an amidine base at elevated temperature to give resin-bound (E)-enones. The reaction is carried out in a polar aprote solvent like DMF or NMP. The reactions are performed 40° C. to 120° C. preferreably at 70° C.-80° C. When the aldol condensation is complete excess of reagent is removed by filtration. The resin is successively washed with the solvent used in the reaction, followed by washing with methanol. The resin bound product can be further dried and analyzed.

Step 4:

This reaction is known (Sadagopan S., Anuradha, K. Tetrahedron Letters. 43, 5181-5183, 2002). The addition of aldehydes to activated double bonds is generally carried out by stirring the aldehyde with a compound that contains an activated dobbelt bond such as a substituted propenone in the presence of a catalyst such as sodium or potassium cyanide or thiazolium salts such as 3,4-dimethyl-5-(2-hydroxyethyl)thiazolium iodide, 3-benzyl-5-(2-hydroxyethyl)-4-methyl-1,3-thiazolium chloride, 3-ethyl-5-(2-hydroxyethyl)-4-methyl-1,3-thiazolium bromide or vitamin B₁. When thiazolium salts are used as catalyst, a non-nucleophilic amine base such as triethyl amine, N,N-diisopropylethylamine or DBU is added. The addition is carried out in a solvent such as dioxane, DMSO, NMP or DMF or a mixture of two or more of these. The reactions are performed between 50° C. to 120° C., preferably between 50° C. to 80° C. When the reaction is complete, excess of reagent is removed by filtration. The resin is successively washed with the solvent used in the reaction, followed by washing with methanol. The resin bound product can be further dried and analyzed.

Step 5:

The reaction is known (The combinatorial index, Ed. Bunin B. A., 1998, Acedemic press, p. 21) and is generally performed by stirring the resin bound intermediate obtained in step 3 with a 50-95% solution of TFA. The final cleavage is carried out in a solvent such as THF, DCM, 1,2 dichloroethane, 1,3-dichloropropane, toluene or a mixture or more of these. The reactions are performed between 0° C. to 80° C., preferably between 20° C. to 40° C. When the reaction is complete the product is removed by filtration. The resin is successively washed with DCM. The product and washings are collected. The solvent is removed and the product is dried in vacou.

The procedure is illustrated in the following example.

### Example 67

### General Procedure (C)

3-{4-[3-(4-Cyclohexylphenyl)-3-oxo-1-(4-trifluoromethoxybenzoyl)propyl]benzoylamino}-propionic acid

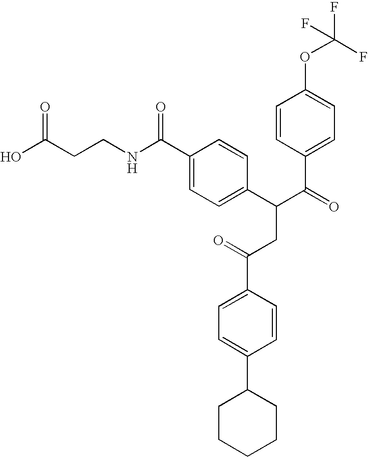

Step 1 and Step 2: Resin bound 3-(4-formylbenzoylamino)propionic acid

3-(4-Formylbenzoylamino)propionic acid resin bound to a Wang resin (loading approximately 0.2-0.8 mmol/g) was synthesized according to the procedure described in WO 00/69810.

Step 3: Preparation of resin bound 3-(4-(3-(4-cyclohexylphenyl)-3-oxopropenyl)benzoylamino)propionic acid

The above resin bound 3-(4-formylbenzoylamino)propionic acid (1 g resin) was suspended in DMF (20 mL) for 30 min and filtered. 4-cyclohexylacetophenone (4.05 g, 20 mmol) was dissolved in DMF (10 mL) and added to the resin. Zinc(II)acetate dihydrate (220 mg, 1 mmol) and 2,2′-bipyridine (156 mg, 1 mmol) was dissolved in DMF (10 mL) and added. DBU (2 mmol) was added and the suspension was shaken at 70° C. for 16 hours. The resin was isolated by filtration and washed with methanol (1×20 mL) and NMP (2×20 mL).

Step 4 and Step 5 Preperation of 3-{4-[3-(4-Cyclohexylphenyl)-3-oxo-1-(4-trifluoromethoxybenzoyl)propyl]benzoylamino}propionic acid

To the above resin bound 3-[4-(3-(4-cyclohexylphenyl)-3-oxopropenyl)benzoylamino)propionic acid was added 3,4-dimethyl-5-(2-hydroxyethyl)thiazolium iodide (2.85 g, 10 mmol) dissolved in NMP (20 mL). 4-(Trifluoromethoxy)benzaldehyde (3.8 g, 10 mmol) was added followed by DBU (10 mmol). The suspension was shaken at 70° C. for 16 hours. The resin was isolated by filtration and washed with methanol (1×20 mL), DCM containing 5% acetic acid (1×20 mL) followed by DCM (3×(20 mL). The resin bound 3-{4-[3-(4-cyclohexylphenyl)-3-oxo-1-(4-trifluoromethoxybenzoyl)propyl]benzoylamino}propionic acid was treated with 50% TFA in DCM (20 mL) for 0.5 hour at 25° C. The mixture was filtered and the resin was washed with DCM (20 mL). The combined filtrates were concentrated in vacuo to afford an oil which was purified on silica gel column eluted with DCM/ethanol (95:5) to afford the title compound.

¹H NMR (CDCl₃): δ 1.15-1.50 (m, 5H), 1.70-1.92 (m, 5H), 2.55 (m, 1H), 2.67 (t, 2H), 3.30 (dd, 1H), 3.70 (q, 3H), 4.17 (dd, 1H), 5.30 (dd, 1H), 6.93 (t, 1H), 7.20 (d, 2H), 7.28 (d, 2H), 7.40 (d, 2H), 7.70 (d, 2H), 7.88 (d, 2H), 8.03 (d, 2H).

The following compounds can be prepared similarly as described above.

### Example 68

### General Procedure (C) 3-{4-[1-(4-Methylbenzoyl)-3-oxo-3-(3-trifluoromethylphenyl)propyl]benzoylamino}propionic acid

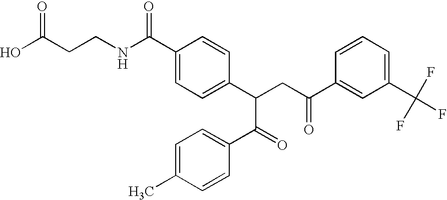

HPLC-MS (Method B)): m/z: 512, Rt: 4.46 min

### Example 69

### General Procedure (C) 3-{4-[1-(4-Methylbenzoyl)-3-oxo-3-(4-trifluoromethoxyphenyl)propyl]benzoylamino}propionic acid

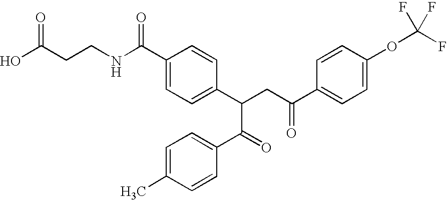

HPLC-MS (Method B)): m/z: 528, Rt: 4.60 min

### Example 70

### General Procedure (C) 3-{4-[1-(4-Bromobenzoyl)-3-oxo-3-(4-trifluoromethoxyphenyl)propyl]benzoylamino}-propionic acid

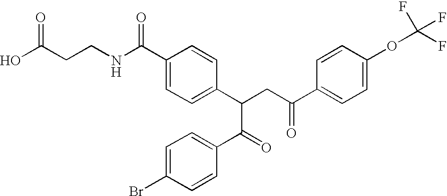

HPLC-MS (Method B)): m/z: 592, Rt: 4.66 min

### Example 71

### General Procedure (C) 3{4-[1-(3,5-Dichlorobenzoyl)-3-oxo-3-(4-trifluoromethoxyphenyl)propyl]benzoylamino}-propionic acid

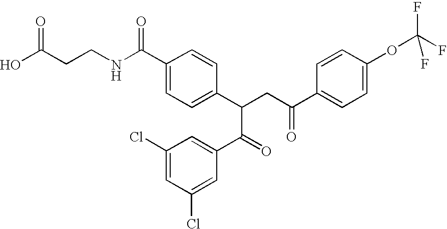

HPLC-MS (Method B)): m/z: 583, Rt: 5.11 min

### Example 72

### General Procedure (C) 3-{4-[1-(Biphenyl-4-carbonyl)-3-oxo-3-(4-trifluoromethoxyphenyl)propyl]benzoylamino}-propionic acid

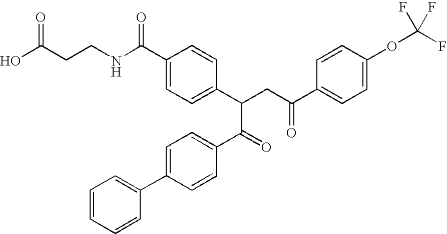

HPLC-MS (Method B)): m/z: 590, Rt: 5.07 min

### Example 73

### General Procedure (C) 3-{4-[1-(4-tert-Butylbenzoyl)-3-oxo-3-3-trifluoromethylphenyl)propyl]benzoylamino}propionic

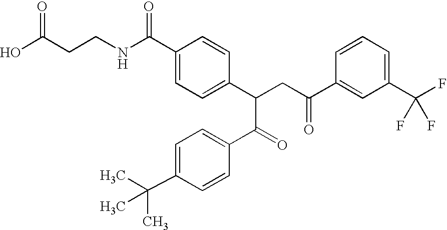

HPLC-MS (Method B)): m/z: 554, Rt: 5.05 min.

### Example 74

### General Procedure (C) 3-{4-[1-(3,5-Dichlorobenzoyl)-3-oxo-3-(3-trifluoromethylphenyl)propyl]benzoylamino}-propionic acid

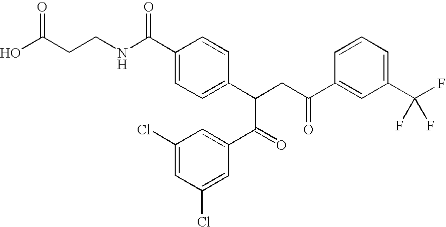

HPLC-MS (Method B)): m/z: 568, Rt: 4.99 min.

### Example 75

### General Procedure (C) 3-{4-[1-(Biphenyl-4-carbonyl)-3-oxo-3-3-trifluoromethylphenyl)propyl]benzoylamino}propionic acid

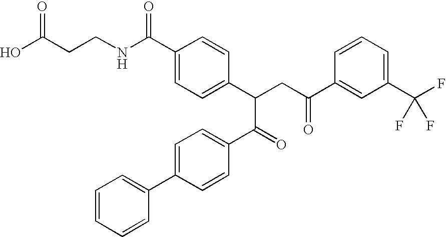

HPLC-MS (Method B)): m/z: 574, Rt: 4.95 min.

### Example 76

### General Procedure (C) 3-{4-[3-(4-Bromophenyl)-1-(4-cyclohexylbenzoyl)-3-oxopropyl]benzoylamino}propionic acid

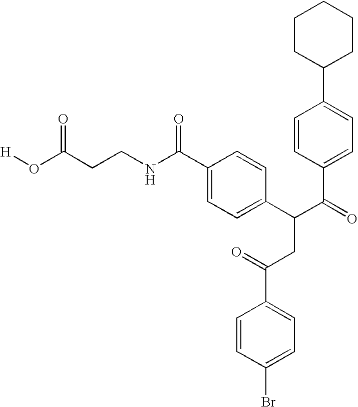

Example 77 General Procedure (C) 3-{4-[1-(4-Cyclohexylbenzoyl)-3-oxo-3-(3-trifluoromethylsulfanylphenyl)propyl]-benzoylamino}propionic acid

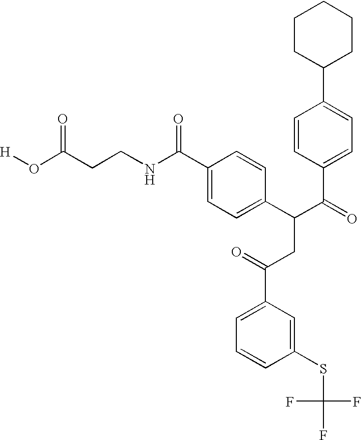

### Example 78

### General Procedure (C) 3-{4-[1-(4-Cyclohexylbenzoyl)-3-(3,4-dichlorophenyl)-3-oxopropyl]benzoylamino}propionic acid

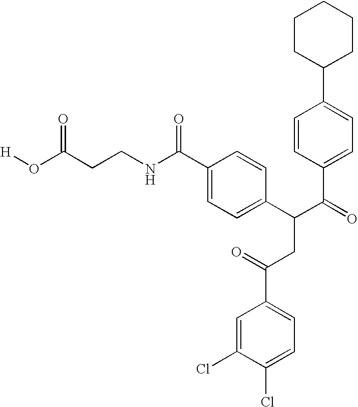

### Example 79

### General Procedure (C) 3-{4-[1-(4-Cyclohexylbenzoyl)-3-oxo-3-(3-trifluoromethylphenyl)propyl]benzoylamino}propionic acid

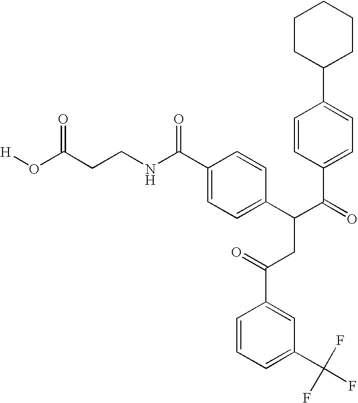

### Example 80

### General Procedure (C) 3-{4-[3-(4-sec-Butylphenyl)-1-(4-cyclohexylbenzoyl)-3-oxopropyl]benzoylamino}propionic acid

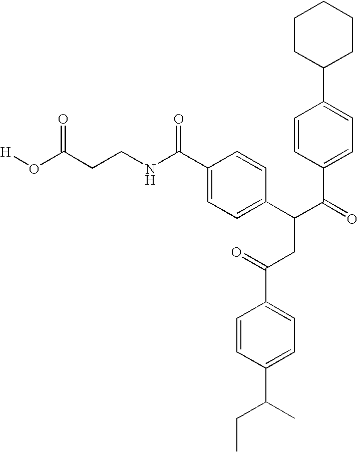

### Example 81

### General Procedure (C) 3-{4-[3-(4-tert-Butylphenyl)-1-(4-cyclohexylbenzoyl)-3-oxopropyl]benzoylamino}propionic acid

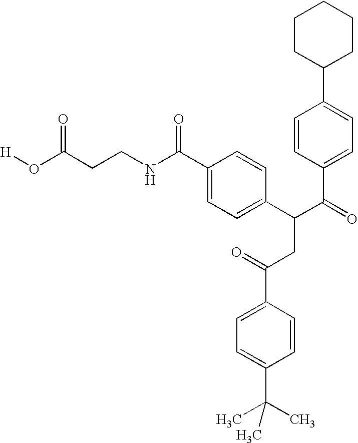

### Example 82

### General Procedure (C)

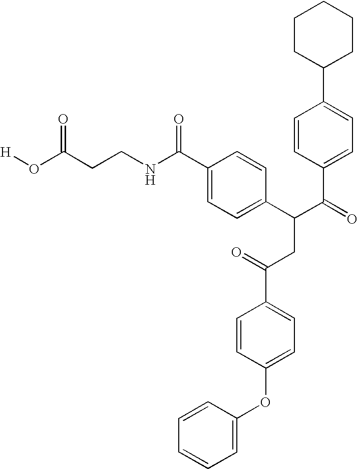

HPLC-MS-(Method (A)): m/z: 604 (M+1) Rt: 6.41 min.

### Example 83

### General Procedure (C) 3-{4-[1-(4-Cyclohexylbenzoyl)-3-oxo-3-(4-trifluoromethylsulfanylphenyl)propyl]benzoylamino}propionic acid

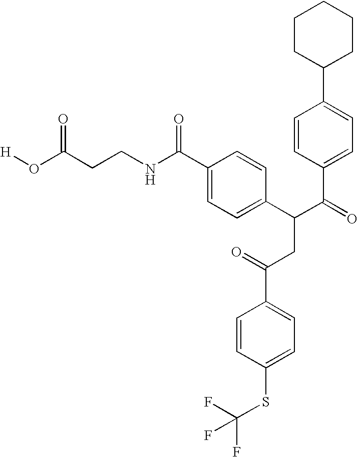

### Example 84

### General Procedure (C) 3-{4-[1-(4-Cyclohexylbenzoyl)-3-(4-cyclohexylphenyl)-3-oxopropyl]benzoylamino}propionic acid

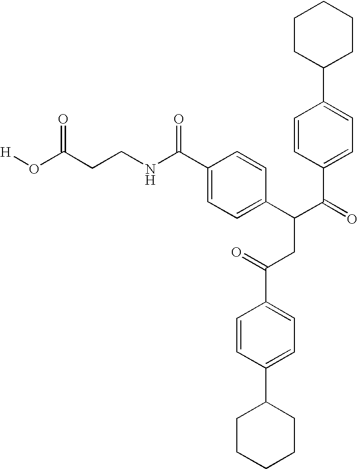

HPLC-MS-(Method (A)): m/z: 594 (M+1) Rt: 7.07 min.

### Example 85

### General Procedure (C) 3-{4-[1-(4-Cyclohexylbenzoyl)-3-oxo-3-(4-trifluoromethoxyphenyl)propyl]benzoylamino}propionic acid

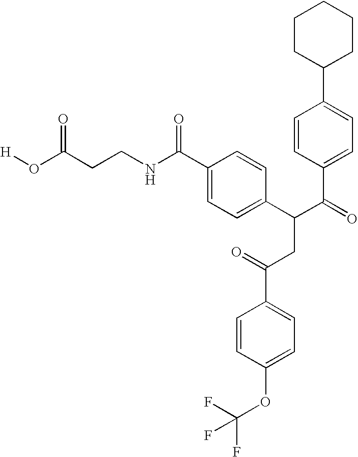

### Example 86

### General Procedure (C)

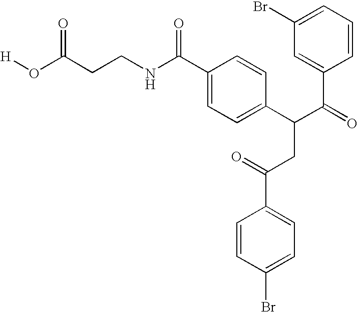

### Example 87

### General Procedure (C) 3-{4-[1-(3-Bromobenzoyl)-3-oxo-3-(3-trifluoromethylsulfanylphenyl)propyl]benzoylamino}propionic acid

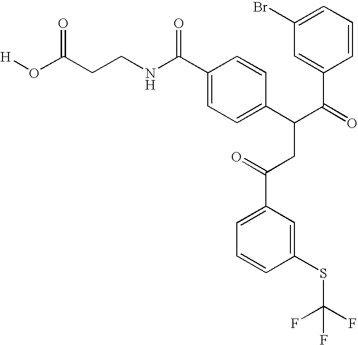

### Example 88

### General Procedure (C) 3-{4-[1-(3-Bromobenzoyl)-3-(3,4-dichlorophenyl)-3-oxopropyl]benzoylamino}propionic acid

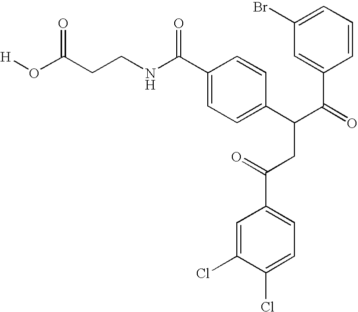

### Example 89

### General Procedure (C)

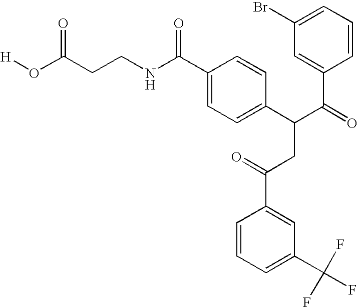

### Example 90

### General Procedure (C) 3-{4-[1-(3-Bromobenzoyl)-3-(4-sec-butylphenyl)-3-oxopropyl]benzoylamino}propionic acid

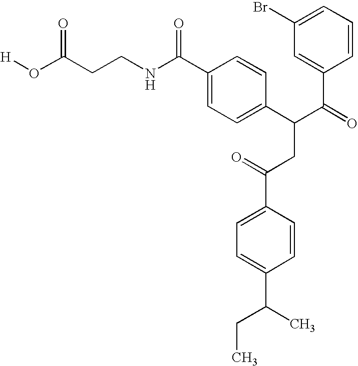

HPLC-MS-(Method (A)): m/z: 565 (M+1) Rt: 5.82 min.

### Example 91

### General Procedure (C) 3-{4-[1-(3-Bromobenzoyl)-3-(4-tert-butylphenyl)-3-oxopropyl]benzoylamino}propionic acid

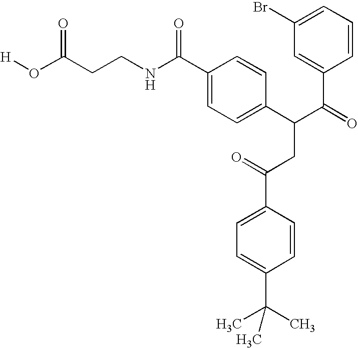

### Example 92

### General Procedure (C) 3-{4-[1-(3-Bromobenzoyl)-3-oxo-3-(4-phenoxyphenyl)propyl]benzoylamino}propionic acid

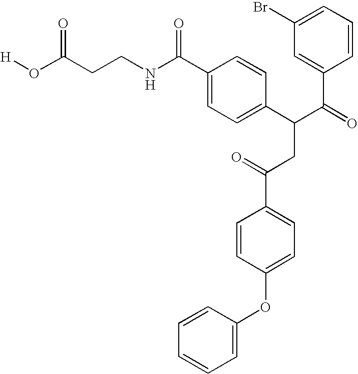

### Example 93

### General Procedure (C) 3-{4-[1-(3-Bromobenzoyl)-3-oxo-3-(4-trifluoromethylsulfanylphenyl)propyl]benzoylamino}propionic acid

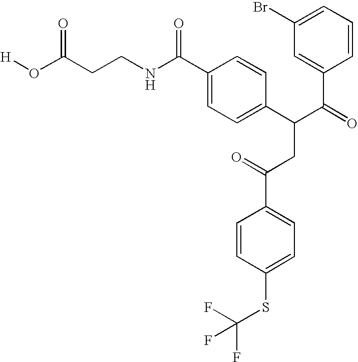

HPLC-MS-(Method (A)): m/z: 608 (M+1) Rt: 5.58 min.

### Example 94

### General Procedure (C) 3-{4-[1-(3-Bromobenzoyl)-3-(4-cyclohexylphenyl)-3-oxopropyl]benzoylamino}propionic acid

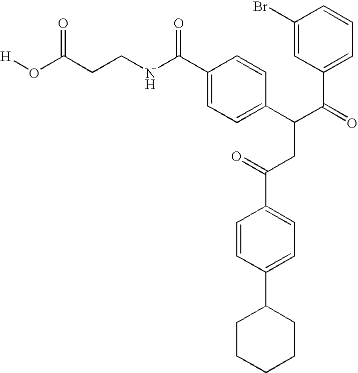

### Example 95

### General Procedure (C) 3-{4-[1-(Biphenyl-4-carbonyl)-3-(4-bromophenyl)-3-oxopropyl]benzoylamino}propionic acid

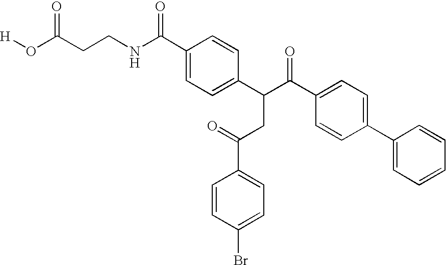

### Example 96

### General Procedure (C) 3-{4-[1-(Biphenyl-4-carbonyl)-3-oxo-3-(3-trifluoromethylsulfanylphenyl)propyl]benzoylamino}propionic acid

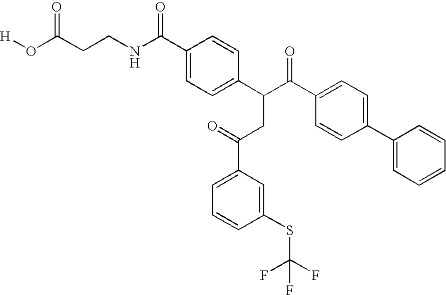

### Example 97

### General Procedure (C) 3-{4-[1-(Biphenyl-4-carbonyl)-3-(3,4-dichlorophenyl)-3-oxopropyl]benzoylamino}propionic acid

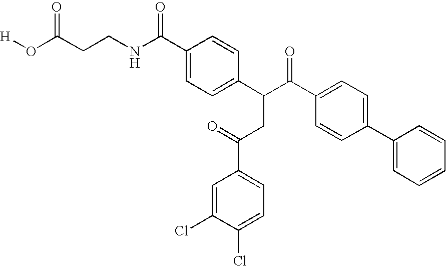

### Example 98

### General Procedure (C) 3-{4-[1-(Biphenyl-4-carbonyl)-3-oxo-3-(3-trifluoromethylphenyl)propyl]benzoylamino}propionic acid

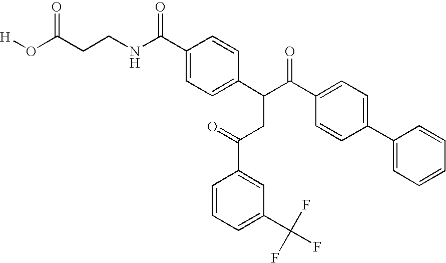

### Example 99

### General Procedure (C) 3-{4-[1-(Biphenyl-4-carbonyl)-3-(4-sec-butylphenyl)-3-oxopropyl]benzoylamino}propionic acid

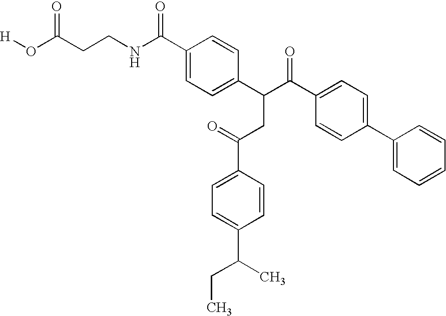

HPLC-MS-(Method (A)): m/z: 562 (M+1) Rt: 6.10 min.

### Example 100

### General Procedure (C) 3-{4-[1-(Biphenyl-4-carbonyl)-3-(4-tert-butylphenyl)-3-oxopropyl]benzoylamino}propionic acid

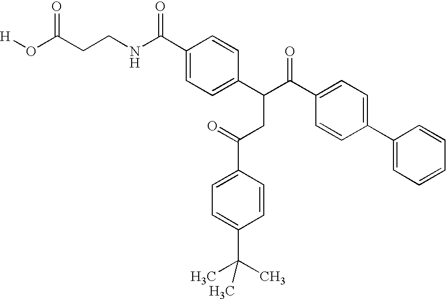

HPLC-MS-(Method (A)): m/z: 562 (M+1) Rt: 6.00 min.

### Example 101

### General Procedure (C) 3-{4-[1-(Biphenyl-4-carbonyl)-3-oxo-3-(4-phenoxyphenyl)propyl]benzoylamino}propionic acid

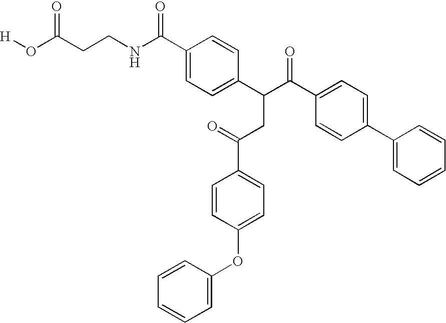

### Example 102

### General Procedure (C) 3-{4-[1-(Biphenyl-4-carbonyl)-3-oxo-3-(4-trifluoromethylsulfanylphenyl)propyl]benzoylamino}propionic acid

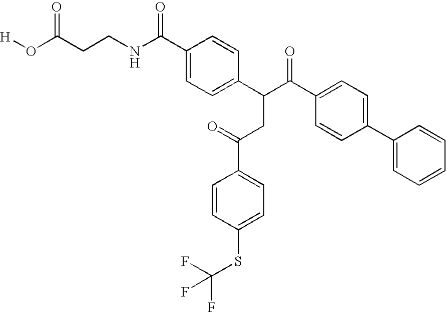

### Example 103

### General Procedure (C) 3-{4-[1-(Biphenyl-4-carbonyl)-3-(4-cyclohexylphenyl)-3-oxopropyl]benzoylamino}propionic acid

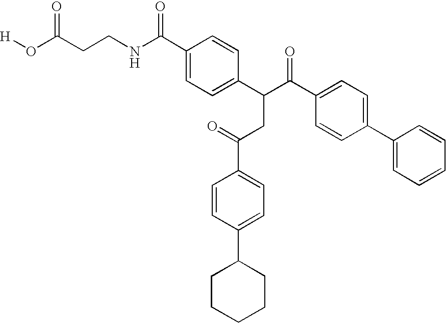

### Example 104

### General Procedure (C)

3-{4-[1-(Biphenyl-4-carbonyl)-3-oxo-344-trifluoromethoxyphenyl)propyl]benzoylamino}propionic acid

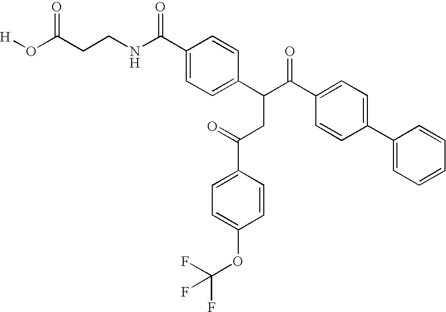

HPLC-MS-(Method (A)): m/z: 612 (M+Na) Rt: 5.70 min.

### Example 105

### General Procedure (C) 3{4-[1-(3,5-Dichlorobenzoyl)-3-oxo-3-(3-trifluoromethylsulfanylphenyl)propyl]benzoylamino}propionic acid

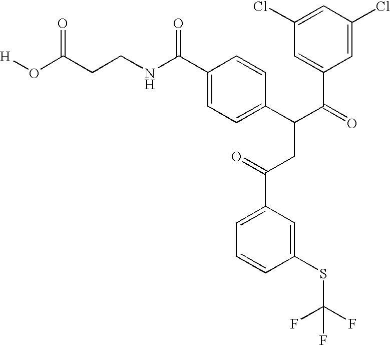

### Example 106

### General Procedure (C) 3{4-[1-(3,5-Dichlorobenzoyl)-3-(3,4-dichlorophenyl)-3-oxopropyl]benzoylamino}propionic acid

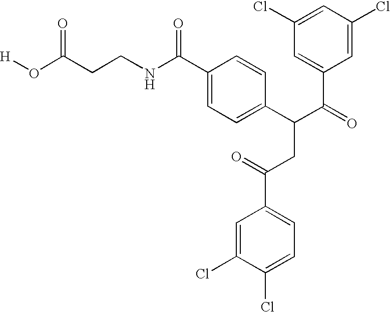

### Example 107

### General Procedure (C) 3-{4-[3-(4-sec-Butylphenyl)-1-(3,5-dichloro-benzoyl)-3-oxopropyl]benzoylamino}propionic acid

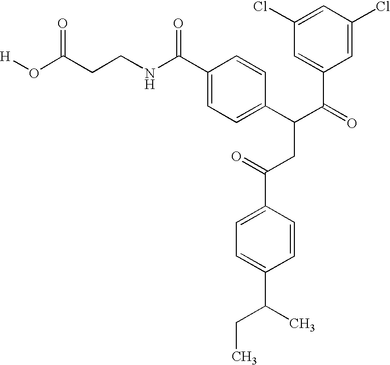

### Example 108

### General Procedure (C) 3-{4-[3-(4-tert-Butylphenyl)-1-(3,5-dichlorobenzoyl)-3-oxopropyl]benzoylamino}propionic acid

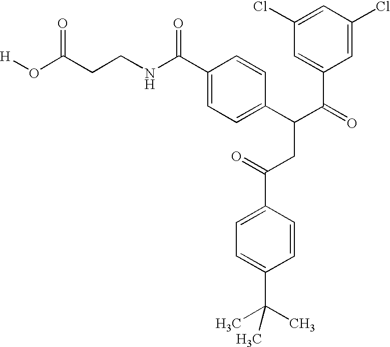

### Example 109

### General Procedure (C) 3-{4-[1-(3,5-Dichlorobenzoyl)-3-oxo-3-(4-phenoxyphenyl)propyl]benzoylamino}propionic acid

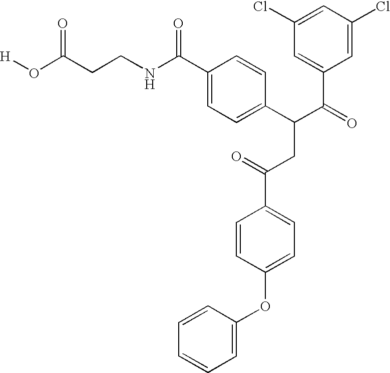

### Example 110

### General Procedure (C) 3-{4-[1-(3,5-Dichlorobenzoyl)-3-oxo-3-(4-trifluoromethylsulfanylphenyl)propyl]benzoylamino}propionic acid

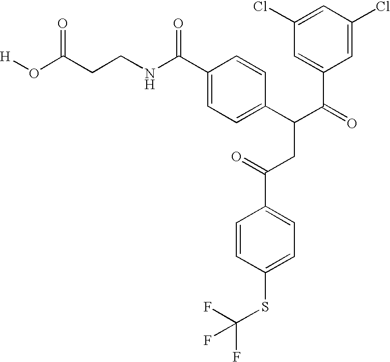

### Example 111

### General Procedure (C) 3{4-[3-(4-Cyclohexylphenyl)-1-(3,5-dichlorobenzoyl)-3-oxopropyl]benzoylamino}propionic acid

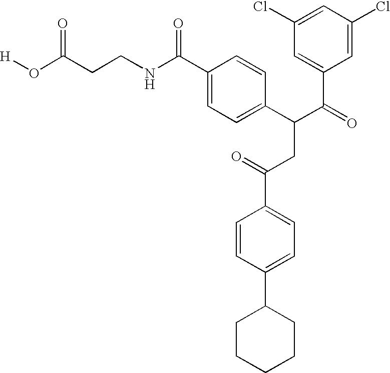

HPLC-MS-(Method (A)): m/z: 581 (M+1) Rt: 6.60 min.

### Example 112

### General Procedure (C) 3{4-[1-(3,5-Dichlorobenzoyl)-3-oxo-3-(4-trifluoromethoxyphenyl)propyl]benzoylamino}propionic acid

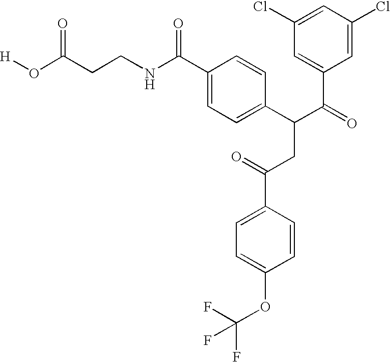

### Example 113

### General Procedure (C) 3-{4-[3-(4-Bromophenyl)-3-oxo-1-(4-trifluoromethoxybenzoyl)propyl]benzoylamino}propionic acid

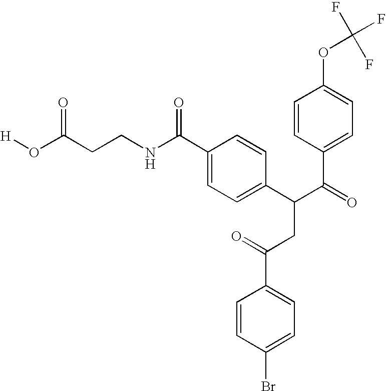

HPLC-MS-(Method (A)): m/z: 594 (M+1) Rt: 5.40 min.

### Example 114

### General Procedure (C) 3-{4-[3-Oxo-1-(4-trifluoromethoxybenzoyl)-3-(3-trifluoromethylsulfanylphenyl)propyl]benzoylamino}propionic acid

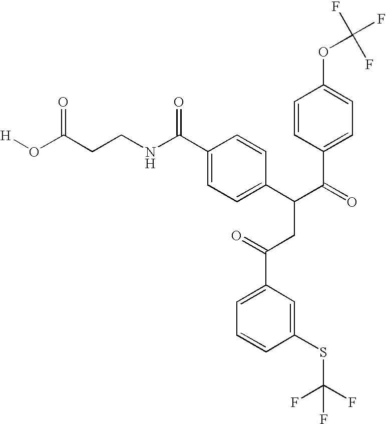

### Example 115

### General Procedure (C) 3{4-[3-(3,4-Dichlorophenyl)-3-oxo-1-(4-trifluoromethoxybenzoyl)propyl]benzoylamino}propionic acid

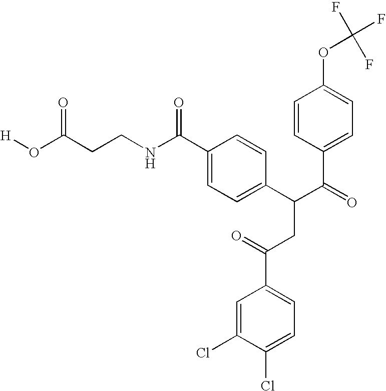

HPLC-MS-(Method (A)): m/z: 583 (M+1) Rt: 5.80 min.

### Example 116

### General Procedure (C) 3-{4-[3-Oxo-1-(4-trifluoromethoxybenzoyl)-3-(3-trifluoromethylphenyl)propyl]benzoylamino}propionic acid

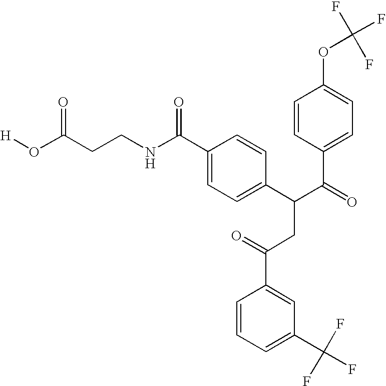

### Example 117

### General Procedure (C) 3{4-[3-(4-sec-Butylphenyl)-3-oxo-1-(4-trifluoromethoxybenzoyl)-propyl]benzoylamino}propionic acid

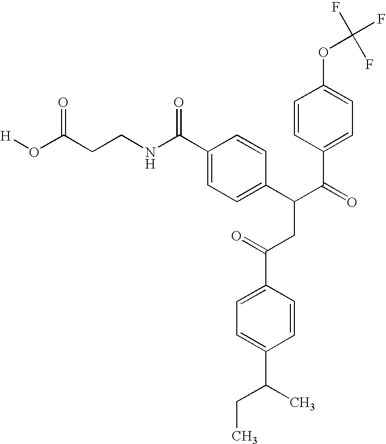

### Example 118

### General Procedure (C) 3-{4-[3-(4-tert-Butylphenyl)-3-oxo-1-(4-trifluoromethoxybenzoyl)propyl]benzoylamino}propionic acid

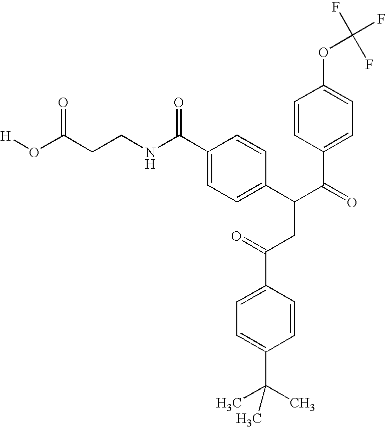

### Example 119

### General Procedure (C) 3-{4-[3-Oxo-3-(4-phenoxyphenyl)-1-(4-trifluoromethoxybenzoyl)-propyl]benzoylamino}propionic acid

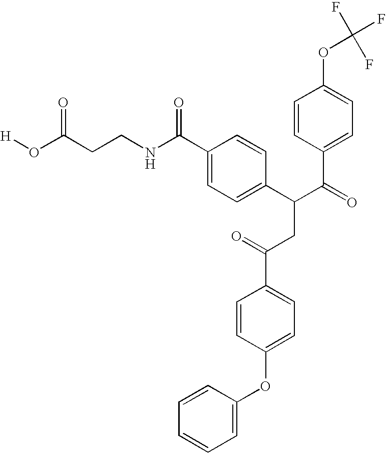

### Example 120

### General Procedure (C) 3-{4-[3-Oxo-1-(4-trifluoromethoxybenzoyl)-3-(4-trifluoromethylsulfanylphenyl)propyl]benzoylamino}propionic acid

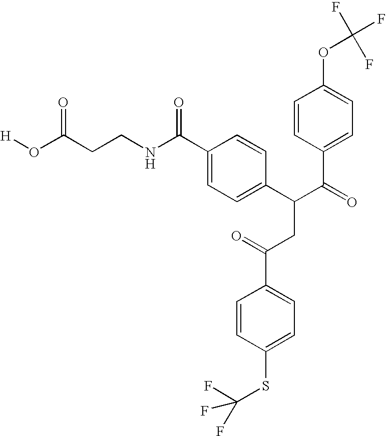

### Example 121

### General Procedure (C) 3-{4-[3-Oxo-1-(4-trifluoromethoxybenzoyl)-3-(4-trifluoromethoxyphenyl)propyl]benzoylamino}propionic acid

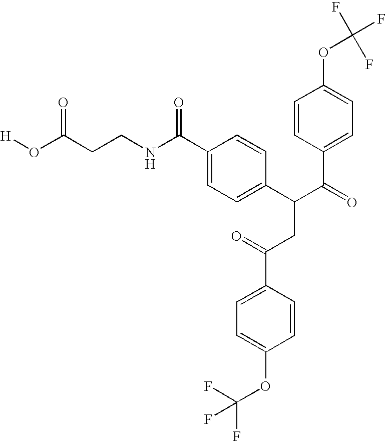

### Example 122

### General Procedure (C) 3-{4-[3-(4-Bromophenyl)-3-oxo-1-(3-trifluoromethoxybenzoyl)propyl]benzoylamino}propionic acid

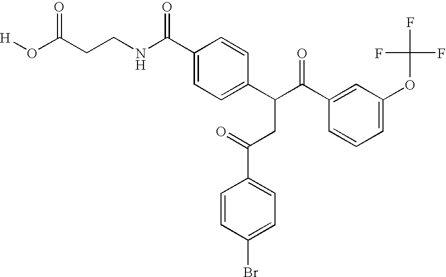

### Example 123

### General Procedure (C) 3-{4-[3-Oxo-1-(3-trifluoromethoxybenzoyl)-3-(3-trifluoromethylsulfanylphenyl)propyl]benzoylamino}propionic acid

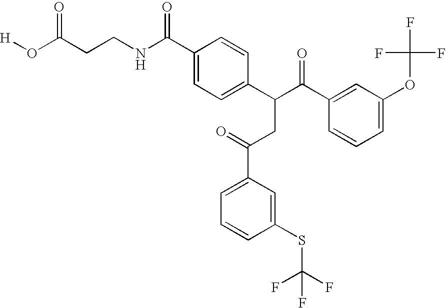

HPLC-MS-(Method (A)): m/z: 614 (M+1) Rt: 5.67 min.

### Example 124

### General Procedure (C) 3-{4-[3-(3,4-Dichlorophenyl)-3-oxo-1-(3-trifluoromethoxybenzoyl)propyl]benzoylamino}propionic acid

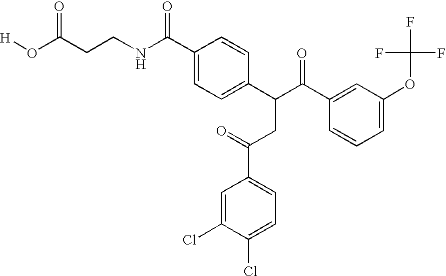

### Example 125

### General Procedure (C) 3-{4-[3-Oxo-1-(3-trifluoromethoxybenzoyl)-3-(3-trifluoromethylphenyl)propyl]benzoylamino}propionic acid

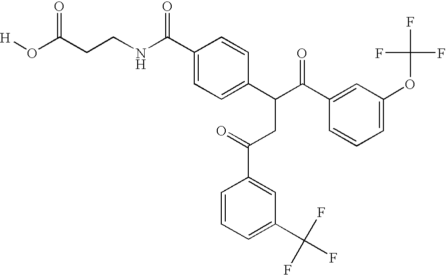

### Example 126

### General Procedure (C) 3-{4-[3-(4-sec-Butylphenyl)-3-oxo-143-trifluoromethoxybenzoyl)propyl]benzoylamino}propionic acid

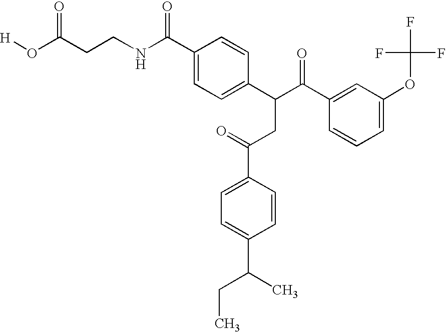

HPLC-MS-(Method (A)): m/z: 570 (M+1) Rt: 5.93 min.

### Example 127

### General Procedure (C) 3-{4-[3-(4-tert-Butylphenyl)-3-oxo-1-(3-trifluoromethoxybenzoyl)propyl]benzoylamino}propionic acid

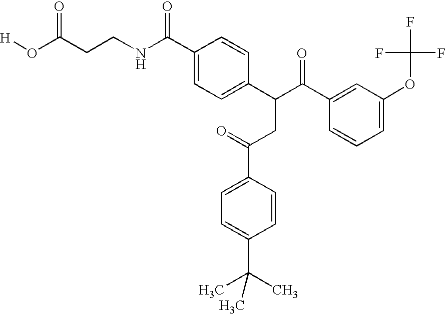

### Example 128

### General Procedure (C) 3{4-[3-Oxo-3-(4-phenoxyphenyl)-1-(3-trifluoromethoxybenzoyl)propyl]benzoylamino}propionic acid

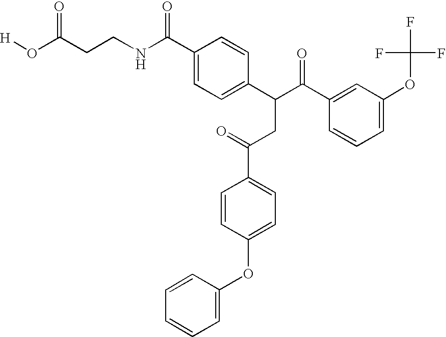

Example 129 General Procedure (C) 3-{4-[3-Oxo-1-(3-trifluoromethoxybenzoyl)-3-(4-trifluoromethylsulfanylphenyl)propyl]benzoylamino}propionic acid

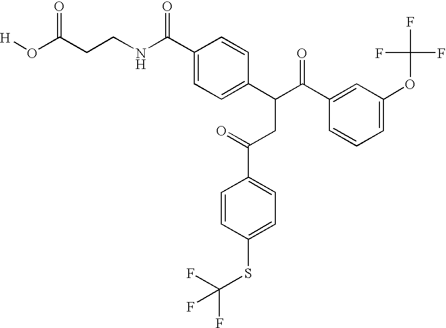

HPLC-MS-(Method (A)): m/z: 614 (M+1) Rt: 5.74 min.

### Example 130

### General Procedure (C) 3-{4-[3-(4-Cyclohexylphenyl)-3-oxo-1-(3-trifluoromethoxybenzoyl)propyl]benzoylamino}propionic acid

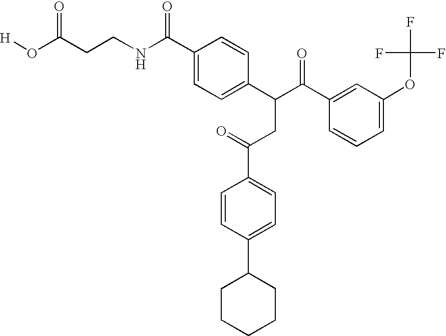

### Example 131

### General Procedure (C) 3-{4-[3-Oxo-1-(3-trifluoromethoxybenzoyl)-3-(4-trifluoromethoxyphenyl)propyl]benzoylamino}propionic acid

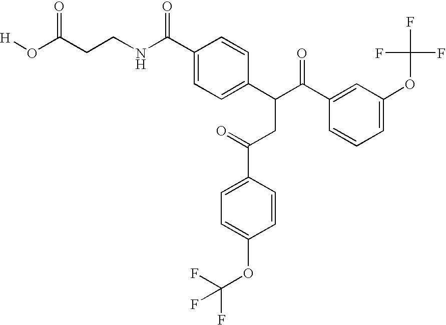

HPLC-MS-(Method (A)): m/z: 598 (M+1) Rt: 5.53 min.

### Example 132

### General Procedure (C) 3-{4-[3-(4-Bromophenyl)-1-(4-tert-butylbenzoyl)-3-oxopropyl]benzoylamino}propionic acid

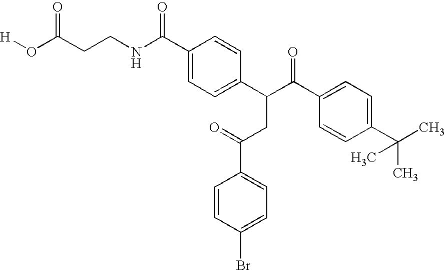

### Example 133

### General Procedure (C) 3-{4-[1-(4-tert-Butylbenzoyl)-3-oxo-3-(3-trifluoromethylsulfanylphenyl)propyl]benzoylamino}propionic acid

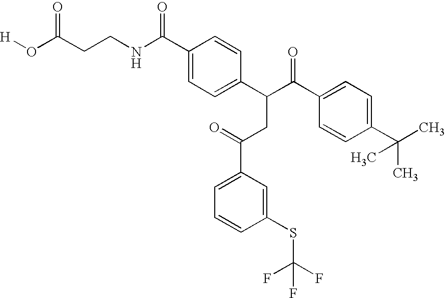

Example 134 General Procedure (C) 3-{4-[1-(4-tert-Butylbenzoyl)-3-(3,4-dichlorophenyl)-3-oxopropyl]benzoylamino}propionic acid

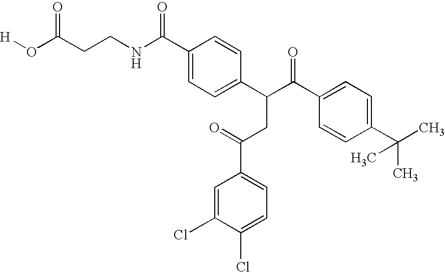

### Example 135

### General Procedure (C) 3-{4-[1-(4-tert-Butylbenzoyl)-3-oxo-3-(3-trifluoromethylphenyl)propyl]benzoylamino}propionic acid

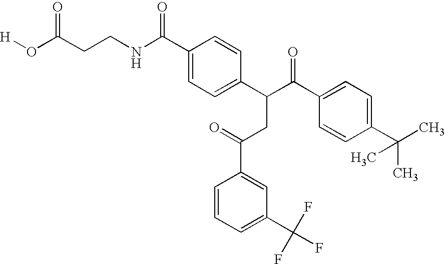

HPLC-MS-(Method (A)): m/z: 554 (M+1) Rt: 5.30 min.

### Example 136

### General Procedure (C) 3-{4-[1-(4-tert-Butylbenzoyl)-3-(4-sec-butylphenyl)-3-oxopropyl]benzoylamino}propionic acid

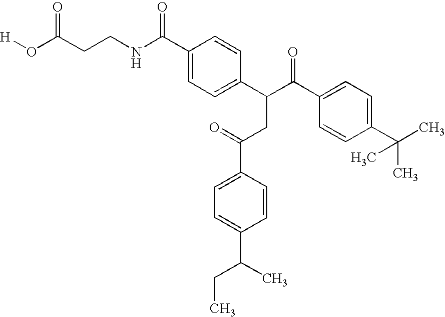

### Example 137

### General Procedure (C)

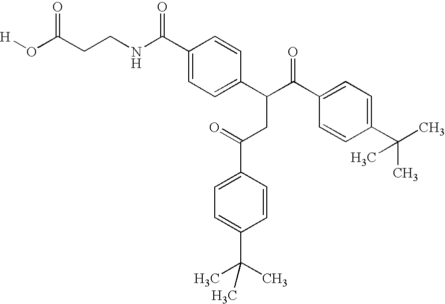

HPLC-MS-(Method (A)): m/z: 542 (M+1) Rt: 6.10 min.

### Example 138

### General Procedure (C) 3-{4-[1-(4-tert-Butylbenzoyl)-3-oxo-3-(4-phenoxyphenyl)propyl]benzoylamino}propionic acid

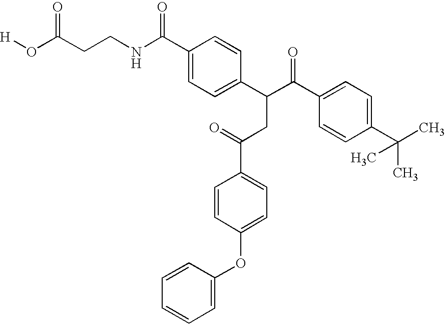

### Example 139

### General Procedure (C) 3-{4-[1-(4-tert-Butylbenzoyl)-3-oxo-3-(4-trifluoromethylsulfanylphenyl)propyl]benzoylamino}propionic acid

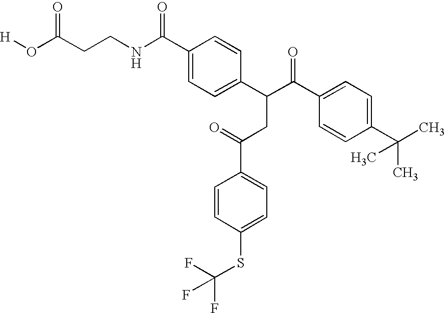

### Example 140

### General Procedure (C) 3-{4-[1-(4-tert-Butylbenzoyl)-3-(4-cyclohexylphenyl)-3-oxopropyl]benzoylamino}propionic acid

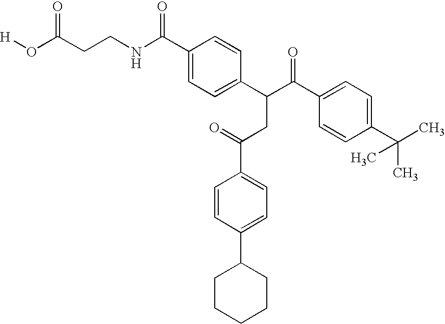

### Example 141

### General Procedure (C) 3{4-[1-(4-tert-Butylbenzoyl)-3-oxo-3-(4-trifluoromethoxyphenyl)propyl]benzoylamino}propionic acid

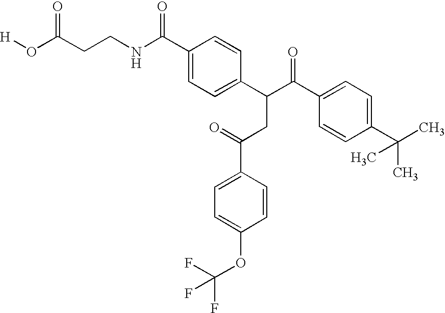

### Example 142

### General Procedure (C) 3-{4-[1-(3,5-Bistrifluoromethylbenzoyl)-3-(4-bromophenyl)-3-oxopropyl]benzoylamino}propionic acid

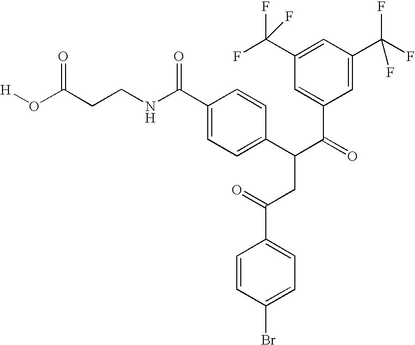

### Example 143

### General Procedure (C) 3-{4-[1-(3,5-Bistrifluoromethylbenzoyl)-3-oxo-3-(3-trifluoromethylsulfanylphenyl)propyl]benzoylamino}propionic acid

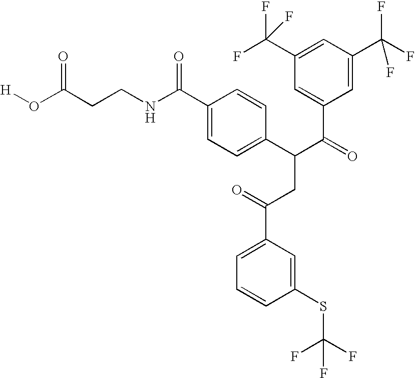

HPLC-MS-(Method (A)): m/z: 666 (M+1) Rt: 6.03 min.

### Example 144

### General Procedure (C) 3-{4-[1-(3,5-Bistrifluoromethylbenzoyl)-3-(3,4-dichlorophenyl)-3-oxopropyl]benzoylamino}propionic acid

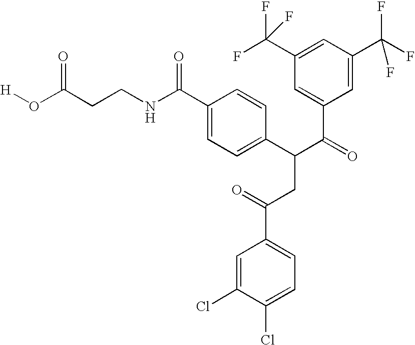

### Example 145

### General Procedure (C) 3{4-[1-(3,5-Bis-trifluoromethylbenzoyl)-3-oxo-3-(3-trifluoromethylphenyl)propyl]benzoylamino}propionic acid

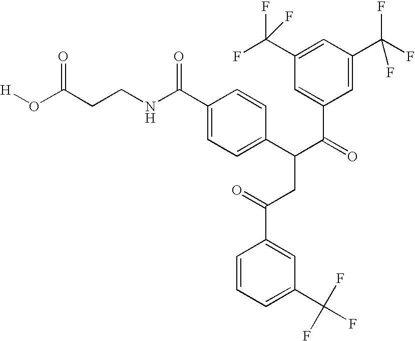

### Example 146

### General Procedure (C) 3-{4-[1-(3,5-Bis-trifluoromethylbenzoyl)-3-(4-sec-butylphenyl)-3-oxopropyl]benzoylamino}propionic acid

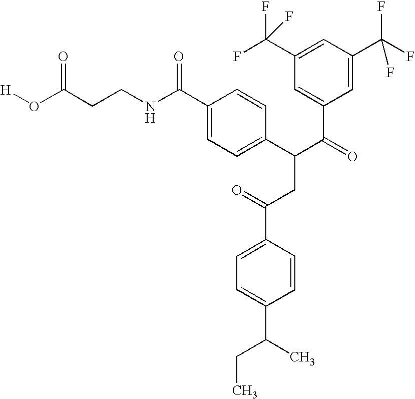

### Example 147

### General Procedure (C) 3-{4-[1-(3,5-Bis-trifluoromethylbenzoyl)-3-(4-tert-butylphenyl)-3-oxopropyl]benzoylamino}propionic acid

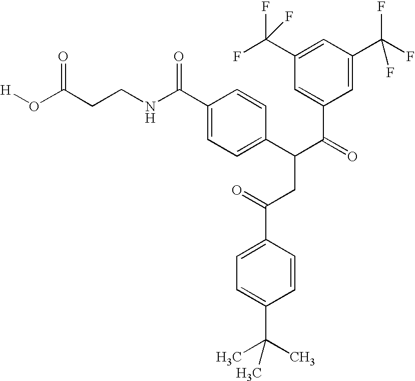

### Example 148

### General Procedure (C) 3-{4-[1-(3,5-Bis-trifluoromethylbenzoyl)-3-oxo-3-(4-phenoxyphenyl)propyl]benzoylamino}propionic acid

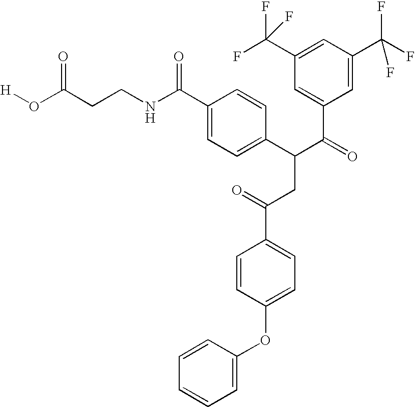

### Example 149

### General Procedure (C) 3-{4-[1-(3,5-Bis-trifluoromethylbenzoyl)-3-oxo-3-(4-trifluoromethylsulfanylphenyl)propyl]benzoylamino}propionic acid

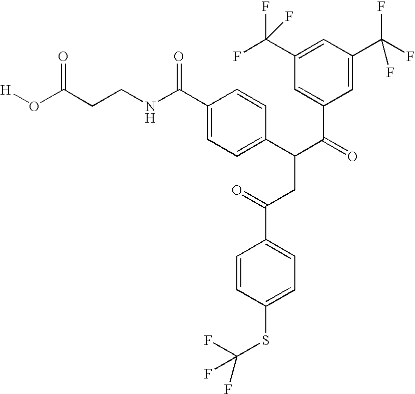

### Example 150

### General Procedure (C) 3-{4-[1-(3,5-Bis-trifluoromethylbenzoyl)-3-(4-cyclohexylphenyl)-3-oxopropyl]benzoylamino}propionic acid

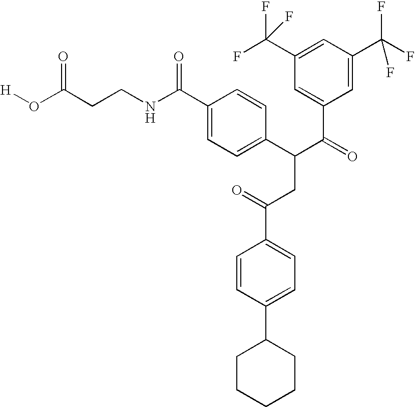

### Example 151

### General Procedure (C) 3-{4-[1-(3,5-Bis-trifluoromethylbenzoyl)-3-oxo-3-(4-trifluoromethoxyphenyl)propyl]benzoylamino}propionic acid

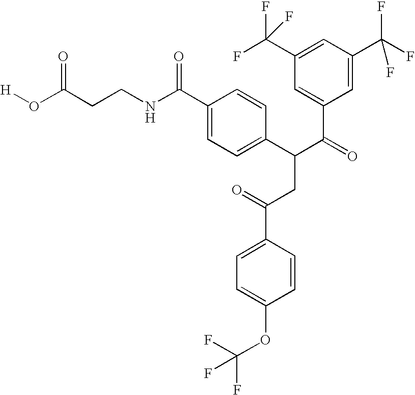

### Example 152

### General Procedure (C) 3-{4-[3-(4-Bromophenyl)-1-(4-isobutylbenzoyl)-3-oxopropyl]benzoylamino}propionic acid

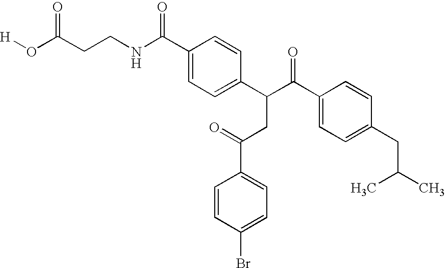

### Example 153

### General Procedure (C) 3-{4-[1-(4-Isobutylbenzoyl)-3-oxo-3-(3-trifluoromethylsulfanylphenyl)propyl]benzoylamino}propionic acid

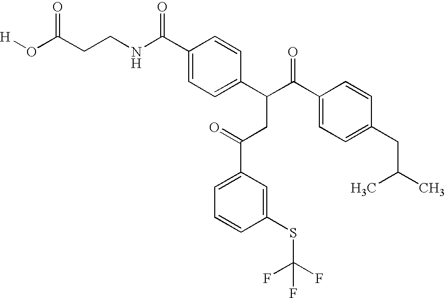

HPLC-MS-(Method (A)): m/z: 586 (M+1) Rt: 6.03 min.

### Example 154

### General Procedure (C) 3-{4-[3-(3,4-Dichlorophenyl)-1-(4-isobutylbenzoyl)-3-oxopropyl]benzoylamino}propionic acid

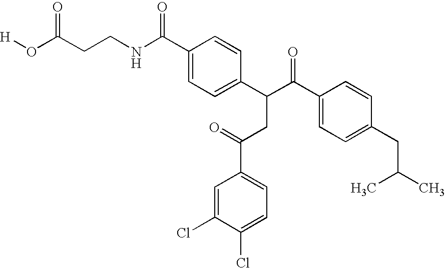

HPLC-MS-(Method (A)): m/z: 554 (M+1) Rt: 5.77 min.

### Example 155

### General Procedure (C) 3-{4-[1-(4-Isobutylbenzoyl)-3-oxo-3-(3-trifluoromethylphenyl)propyl]benzoylamino}propionic acid

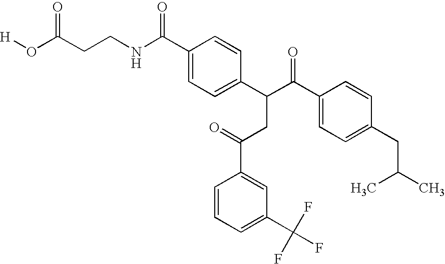

HPLC-MS-(Method (A)): m/z: 554 (M+1) Rt: 1.07 min.

### Example 156

### General Procedure (C) 3-{4-[3-(4-sec-Butylphenyl)-1-(4-isobutylbenzoyl)-3-oxopropyl]benzoylamino}propionic acid

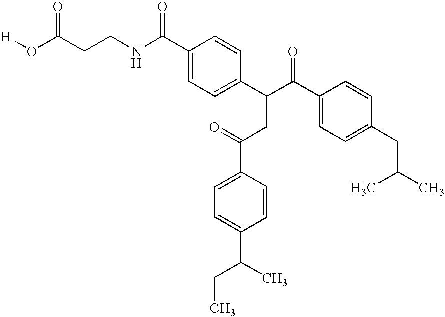

### Example 157

### General Procedure (C) 3-{4-[3-(4-tert-Butylphenyl)-1-(4-isobutylbenzoyl)-3-oxopropyl]benzoylamino}propionic acid

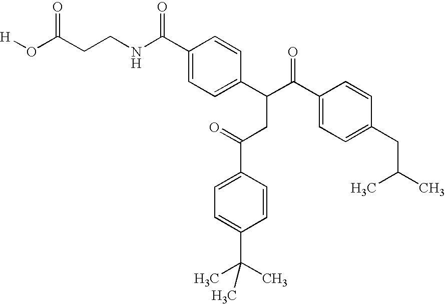

### Example 158

### General Procedure (C) 3{4-[1-(4-Isobutylbenzoyl)-3-oxo-3-(4-phenoxyphenyl)propyl]benzoylamino}propionic acid

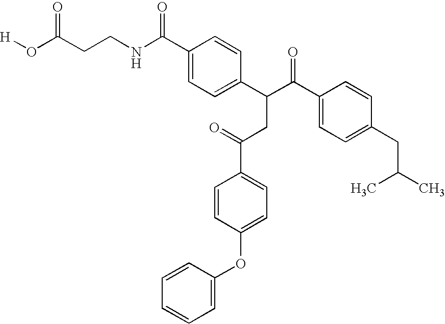

### Example 159

### General Procedure (C) 3-{4-[1-(4-Isobutylbenzoyl)-3-oxo-3-(4-trifluoromethylsulfanylphenyl)propyl]benzoylamino}propionic acid

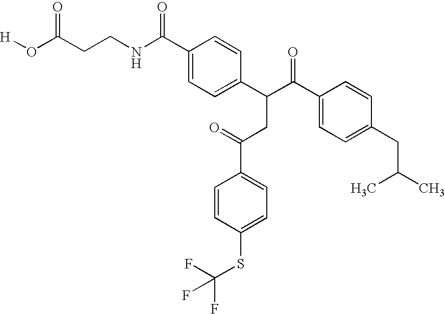

### Example 160

### General Procedure (C) 3-{4-[3-(4-Cyclohexylphenyl)-1-(4-isobutylbenzoyl)-3-oxopropyl]benzoylamino}propionic acid

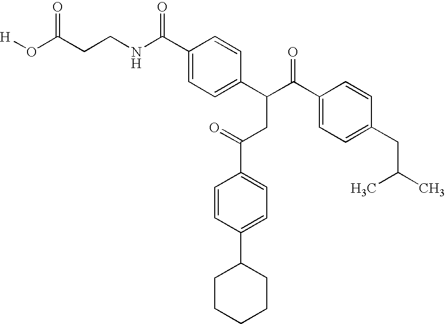

### Example 161

### General Procedure (C) 3-{4-[1-(4-Isobutylbenzoyl)-3-oxo-3-(4-trifluoromethoxyphenyl)propyl]benzoylamino}propionic acid

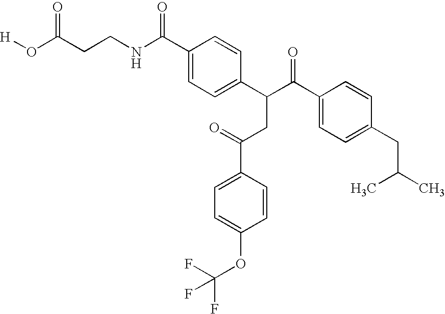

### Example 162

### Z-3-{4-[3-(4-Cyclohexylphenyl)-3-oxo-1-(4-trifluoromethoxybenzoyl)propenyl]benzoylamino}propionic acid

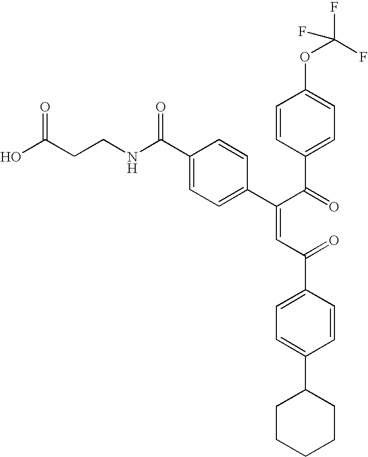

Iodine (153 mg, 0.6 mmol) was dissolved in THF (4 mL) and DBU (0.271 mL, 1.8 mmol) was added. This solution was added to 3-{4-[3-(4-cyclohexylphenyl)-3-oxo-1-(4-trifluoromethoxybenzoyl)propyl]benzoylamino}propionic acid (300 mg, 0.5 mmol) and stirred at room temperature for 30 min. The solution was diluted with diethyl ether (30 mL) and washed with a sodium sulfite solution (2% in water, 2×30 mL). The organic phase was washed with 1 N HCl (30 mL), dried (Na₂SO₄) and solvent removed by evaporation. A foam appeared which was redisolved in toluene (30 mL) and 4 drops of concentrated aq. HCl was added. The mixture was heated to reflux for 30 min, and evaporated give the title compound. ¹H NMR (CDCl₃): δ 1.10-1.50 (m, 5H), 1.60-1.95 (m, 5H), 2.55 (m, 1H), 2.67 (t, 2H), 7.08 (br s, 1H), 7.20 (d, 2H), 7.38 (d, 2H), 7.60 (d, 2H), 7.68 (s 1H), 7.78 (d, 2H), 7.90 (d, 2H), 7.95 (d, 2H).

General Procedure D:

General procedure (D) may be used for solid phase preparation of compounds of general formula (Ic):

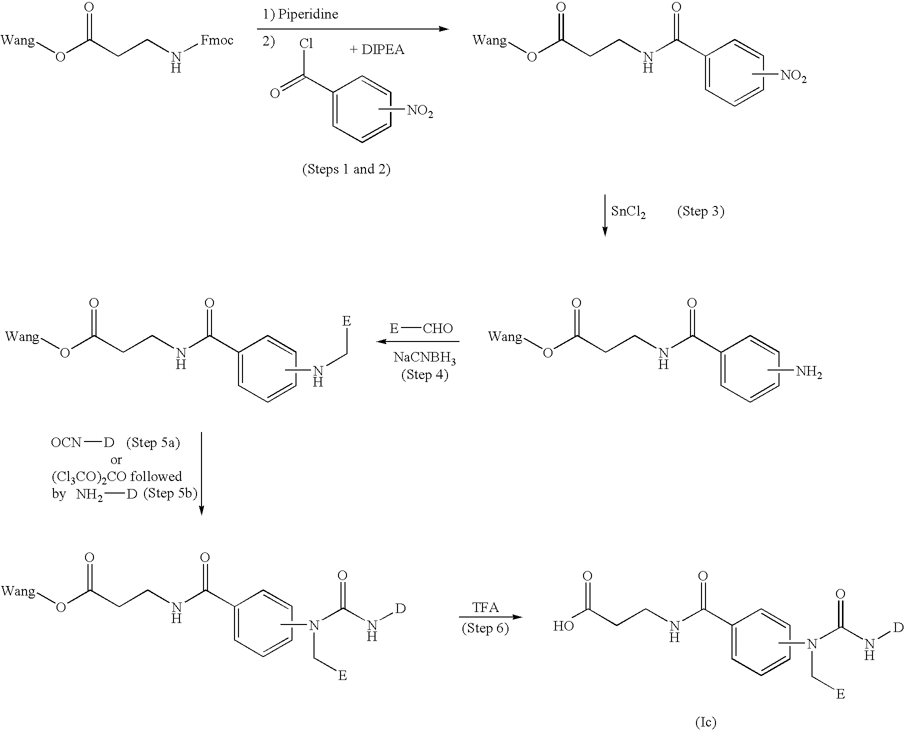

wherein E and D independently are aryl or heteroaryl and are both optionally substituted as previously described.

Steps 1 to 2:

These steps are analogous to the corresponding steps described in WO 00/69810 and WO 02/00612.

Step 3:

The reduction of aromatic nitro groups on solid phase is generally known (F. Z. Dörwald, “Organic Synthesis on Solid Phase”, 1^{st} Edition Wiley-VCH: Weinheim, 2000, p. 246-247), and is performed using an excess of tin (II) chloride dihydrate in a polar organic solvent such as DMF or NMP. The reaction is performed at 20-100° C., preferable at ambient or slightly elevated temperature.

Step 4:

This reaction is generally known (F. Z. Döwald, “Organic Synthesis on Solid Phase”, 1^{st} Edition Wiley-VCH: Weinheim, 2000, p. 239-241), and is achieved by using an excess of aldehyde, sodium cyano borohydride and a proton source such as acetic acid. The reaction is performed at 20-100° C. preferable at 40-80° C. in a polar organic solvent such as DMF or NMP.

Steps 5a:

This reaction is known (see WO 00/69810 and WO 02/00612), and is generally performed by reacting solid phase tethered amine with alkyl or aryl isocyanates in organic solvents. The reaction is performed at 20-100° C. preferable at 20-40° C. in an aprotic organic solvent such as toluene, DCP or DCM depending on the temperature.

Steps 5b:

The formation of urea can alternatively be performed via chlorocarbamoylation followed by reaction with amines. The formation of carbamoyl chlorides from amines tethered on solid support is a know reaction (Wang, G. T. et al.; Tetrahedron Lett, 1997, 38 (11), 1895-1898, Scicinski, J. J.; Barker, M. D.; Murray, P. J.; Jarvie, E. M.; Bioorg Med Chem Lett 1998, 8 (24), 3609-3614), and is generally performed by adding phosgene (or a synthetic equivalent such as bis(trichloromethoxy)carbonate or trichloromethoxycarbonyl chloride) to resin bound primary or secondary amine in the presence of base. As base, an organic amine such as triethylamine, pyridine, or DIPEA can be used. The reaction is preferably performed at 0-20° C., in an inert aprotic solvent such as DCM, toluene, THF or the like.

The subsequent urea formation from carbamoyl chloride and amine is also known (Wang, G. T. et al.; Tetrahedron Lett 1997, 38 (11), 1895-1898) and is performed by reacting carbamoyl chlorides with aliphatic amines in the presence of base. As base, an organic amine such as triethylamine, pyridine, DIPEA or 1,4-diazabicyclo[2.2.2.]octane (DABCO) can be used. The reaction is performed at 0-80° C., preferably at ambient temperature in an inert aprotic polar solvent such as DMF, THF or NMP.

Step 6: Cleavage from Resin

This step is analogous to the corresponding transformations described in WO 00/69810 and WO 02/00612.

The general procedure (D) is further illustrated in the following example:

### Example 163

### General Procedure (D)

3-{4-[3-(3,5-Bis[trifluoromethyl]phenyl)-1-(4-cyclohexylbenzyl)ureido]benzoylamino}propionic acid

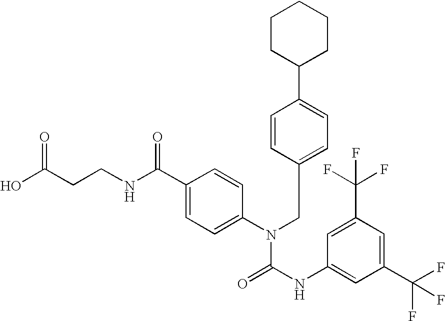

Step 1: Fmoc-β-Ala-Wang resin (5.0 g, 0.31 mmol/g, 1.55 mmol) was treated with piperidine (20% in NMP, 20 ml) for 30 min., and the resin was drained. This was repeated once. The resin was then washed with DMF (5×).

Step 2: A solution of DIPEA (3 ml) in NMP (17 ml) was added, followed by slow addition of a solution of p-nitrobenzoylchloride (2.88 g; 15.5 mmol, 10 eq.) in NMP (20 ml). The mixture was shaken for 3 h, then drained. The resin was washed with DMF (5×).

Step 3: Then a solution of SnCl₂. 2H₂O (10.5 g; 46.5 mmol, 30 eq.) in NMP (30 ml) was added. The mixture was shaken at room temperature for 16 h. The resin was drained and washed with DMF (3×) and DCM (10×), then dried under vacuum for 16 h. Yield: 5.20 g.

Step 4: Dry resin (100 mg; 54 mmol; 0.54 mmol/g) prepared as described above, was swelled in DCM for 30 min. 4-Cyclohexylbenzaldehyde (188.2 mg; 1 mmol) dissolved in DMF (1 ml) was added followed by a solution of sodium cyano borohydride (138 mg; 2 mmol) in DMF—acetic acid (1.2 ml, 5:1). The reaction mixture was heated to 80° C. over night. The resin was drained for solvent and reactants, and subsequently washed with MeOH (3×), DMF (5×) and DCM (4×) to give resin bound 3-{4-[(4-cyclohexylphenylmethyl)amino]benzoylamino}propionic acid.

Step 5a: Resin bound 3-{4-[(4-cyclohexylphenylmethyl)amino]benzoylamino}propionic acid (100 mg; 0.54 mmol/g) was suspended in DCP (1.0 ml) and 3,5-bis(trifluoromethyl)phenyl isocyanate (255 mg, 1 mmol) was added. The reaction mixture was stirred at room temperature for 48 h, then washed with DMF (3×) and DCM (10×).

Step 6: The product was cleaved from the resin using 50% TFA in DCM. Solvent was removed by speed evacuation to give 10-12 mg of pure title material. HPLC-MS (Method (B)): m/z: 636 (M+1), Rt: 5.09 min

### Example 164

### General procedure (D)

3-{4-[3-[1(S)-(4-Chlorophenyl)ethyl]-1-(4-cyclohexylbenzyl)ureido]benzoylamino}propionic acid

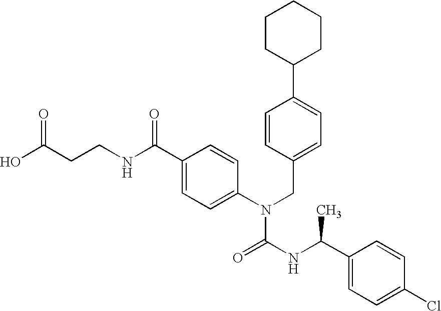

Step 5b: Resin bound 3-{4-[(4-cyclohexylphenylmethyl)amino]benzoylamino}propionic acid (100 mg; 0.54 mmol/g), prepared as in the above example (step 1-4) was suspended in DCM (500 ul) and DIPEA (100 ul) was added. A solution of bis(trichloromethyl)carbonate (44 mg; 0.15 mmol) in DCM (500 ul) was then added. The reaction was shaken for 60 min. Then the solvent was drained and the resin was washed with DCM (4×), to give resin bound 3-{4-[chlorocarbonyl-(4-cyclohexylphenylmethyl)amino]benzoylamino}propionic acid.

To resin bound 3-{4-[chlorocarbonyl-(4-cyclohexylphenylmethyl)amino] benzoylamino}propionic acid, was added a solution of (S)-1-(4-chlorophenyl)ethylamine (78 mg; 0.5 mmol) in DMF (500 ul) followed by a solution of DABCO (100 mg) in DMF (500 ul). The reaction was stirred overnight at ambient temperature, then washed with DMF (3×), 10% HOAc-MeOH (3×) and DCM (10×).

Step 6: The product was cleaved from the resin using 50% TFA in DCM. Solvent was removed by speed evacuation to give 10-12 mg of pure title compound.

HPLC-MS (Method (B)): m/z: 563 (M+1), Rt: 5.09 min

In a similar manner, the following compounds were made:

### Example 165

### General Procedure (D, via step 5b)

3-{4-[3-[1 (R)-(4-Chlorophenyl)ethyl]-1-(4-cyclohexylbenzyl)ureido]benzoylamino}propionic acid

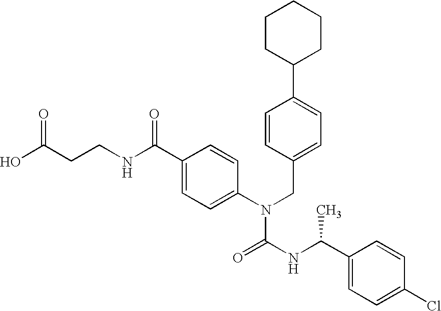

HPLC-MS-(Method (B)): m/z 563 (M+1) Rt: 5.08 min.

### Example 166

### General Procedure (D, via step 5a) 3-{4-[3-(3-Cyanophenyl)-1-(4-cyclohexylbenzyl)ureido]benzoylamino}propionic acid

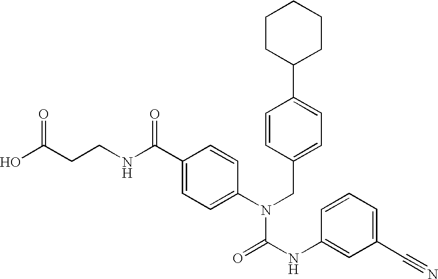

HPLC-MS-(Method (B)): m/z 525 (M+1) Rt: 4.79 min.

### Example 167

### General Procedure (D, via step 5a) 3-{4-[1-(4-Cyclohexylbenzyl)-3-(4-isopropylphenyl)ureido]benzoylamino}propionic acid

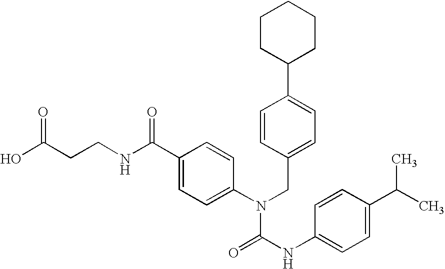

HPLC-MS-(Method (B)): m/z 542 (M+1) Rt: 5.41 min.

### Example 168

### General Procedure (D, via step 5a)

3-{4-[3-(4-Chloro-3-trifluoromethylphenyl)-1-(4-cyclohexylbenzyl)ureido]benzoylamino}-propionic acid

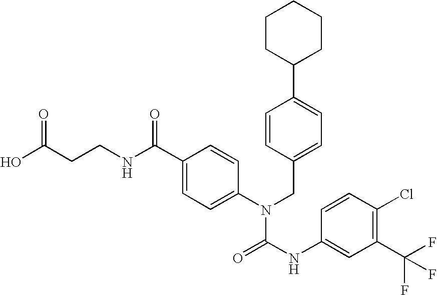

HPLC-MS-(Method (B)): m/z 603 (M+1) Rt: 5.64 min.

### Example 169

### General Procedure (D, via step 5a) 3-{4-[1-(4-Cyclohexylbenzyl)-3-(2,2,3,3-tetrafluoro-2,3-dihydrobenzo[1,4]dioxin-6-yl)ureido]-benzoylamino}propionic acid

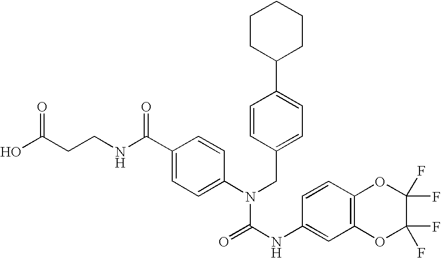

HPLC-MS-(Method (B)): m/z 630 (M+1) Rt: 5.68 min.

### Example 170

### General Procedure (D, via step 5a)

3-{4-[1-(4-Cyclohexylbenzyl)-3-(4-trifluoromethylsulfanylphenyl)ureido]benzoylamino}-propionic acid

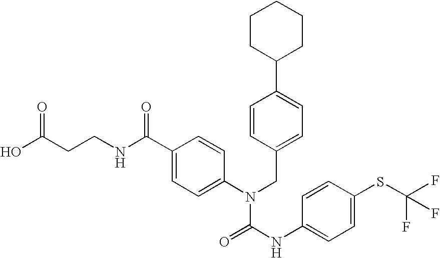

HPLC-MS-(Method (B)): m/z 600 (M+1) Rt: 5.69 min.

### Example 171

### General Procedure (D, via step 5b)

3-{4-[1-Biphenyl-4-ylmethyl-3-(4-trifluoromethoxybenzyl)ureido]benzoylamino}propionic acid

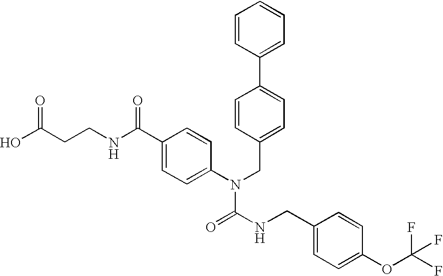

HPLC-MS-(Method (B)): m/z 592 (M+1) Rt: 4.78 min.

### Example 172

### General Procedure (D, via step 5b)

3{4-[1-(4-Cyclohexylbenzyl)-3-(4-trifluoromethoxybenzyl)ureido]benzoylamino}propionic acid

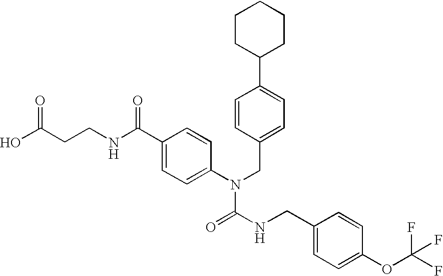

HPLC-MS-(Method (B)): m/z 598 (M+1) Rt: 5.28 min.

### Example 173

### General Procedure (D, via step 5b)

3-{4-[1-(4-Cyclohexylbenzyl)-3-(4-trifluoromethoxybenzyl)ureido]benzoylamino}propionic acid

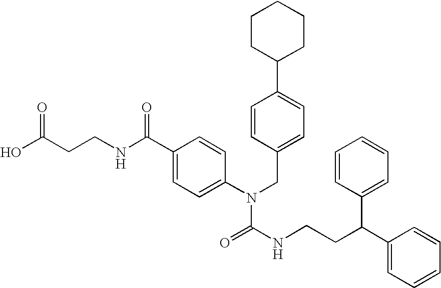

HPLC-MS-(Method (B)): m/z 619 (M+1) Rt: 5.72 min.

### Example 174

### General Procedure (D, via step 5a)

3-{4-[1-(4-Chlorobenzyl)-3-(4-cyclohexylphenyl)ureido]benzoylamino}propionic acid

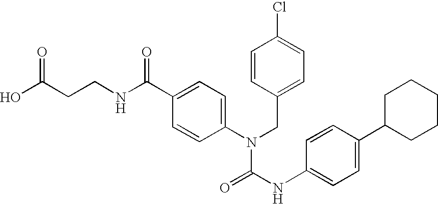

HPLC-MS (Method B): m/z: 535 (M+1); Rt=5.70 min.

### Example 175

### General Procedure (D, via step 5a)

3-{4-[1-(4-Chlorobenzyl)-3-(3,5-dichlorophenyl)ureido]benzoylamino}propionic acid

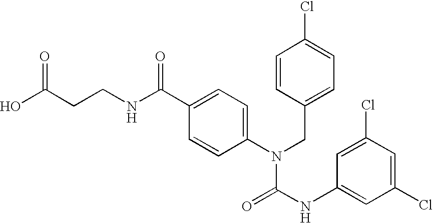

HPLC-MS (Method B): m/z: 521 (M+1); Rt=5.33 min.

### Example 176

### General Procedure (D, via step 5a)

3-{4-[1-(4-Bromobenzyl)-3-(4-cyclohexylphenyl)ureido]benzoylamino}propionic acid

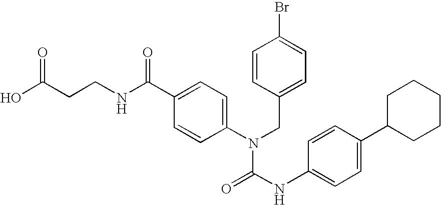

HPLC-MS (Method B): m/z: 579 (M+1); Rt=5.80 min.

### Example 177

### General Procedure (D, via step 5a)

3-{4-[1-(4-Bromobenzyl)-3-(3,5-dichlorophenyl)ureido]benzoylamino}propionic acid

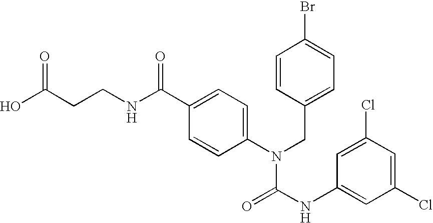

HPLC-MS (Method B): m/z: 566 (M+1); Rt=5.57 min.

### Example 178

### General Procedure (D, via step 5a)

3-{4-[3-(4-Cyclohexylphenyl)-1-(4-trifluoromethoxybenzyl)ureido]benzoylamino}propionic acid

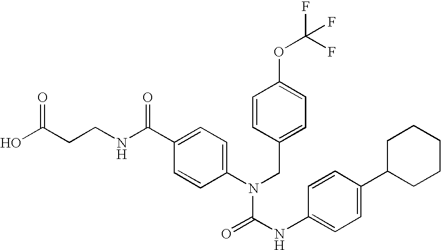

HPLC-MS (Method B): m/z: 584 (M+1); Rt=5.97 min.

### Example 179

### General Procedure (D, via step 5a)

3-{4-[3-(3,5-Dichlorophenyl)-1-(4-trifluoromethoxybenzyl)ureido]benzoylamino}propionic acid

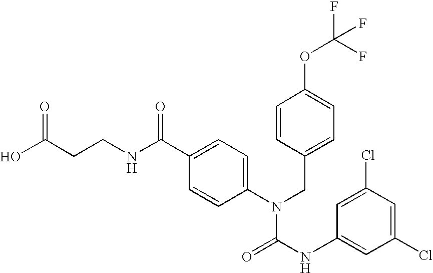

HPLC-MS (Method B): m/z: 570 (M+1); Rt=5.57 min.

### Example 180

### General Procedure (D, via step 5b)

3-{3-[3-[1 (R)-(4-Chlorophenyl)ethyl]-1-(4-cyclohexylbenzyl)ureido]benzoylamino}propionic acid

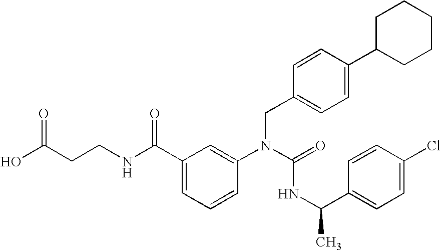

HPLC-MS-(Method (B)): m/z 563 (M+1) Rt: 5.13 min.

### Example 181

### General Procedure (D, via step 5b)

3-{3-[3-[1 (S)-4-Chlorophenyl)ethyl]-1-(4-cyclohexyl-benzyl)ureido]benzoylamino}propionic acid

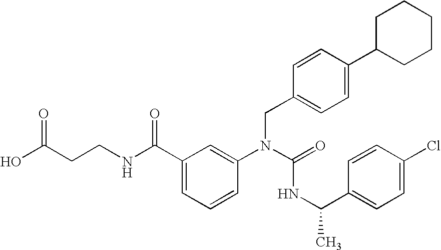

HPLC-MS-(Method (B)): m/z 563 (M+1) Rt: 5.13 min.

### Example 182

### General Procedure (D, via step 5a)

3-{3-[1-(4-Cyclohexylbenzyl)-3-(4-trifluoromethoxyphenyl)ureido]benzoylamino}propionic acid

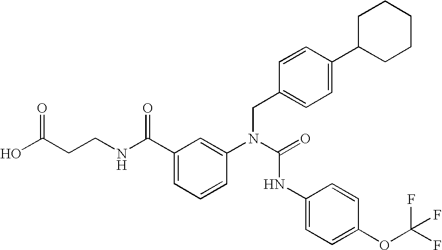

HPLC-MS-(Method (B)): m/z 584 (M+1) Rt: 5.41 min.

General Procedure E:

General procedure (E) may be used for solution phase preparation of compounds of general formula (Ic):

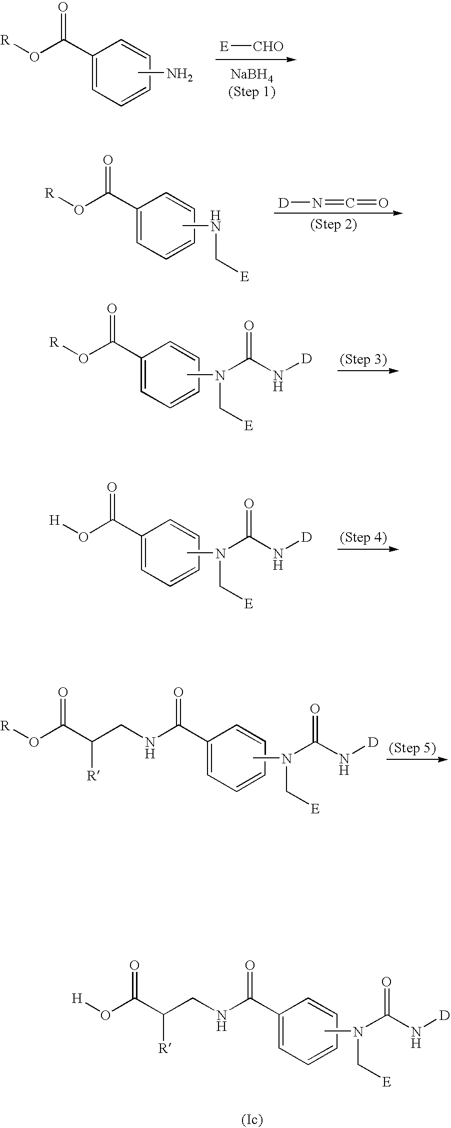

wherein

R are hydrogen or C₁₋₆-alkyl,

R′=H or OH, and E and D independently are aryl or heteroaryl and are both optionally substituted as defined above

Step 1:

The reductive amination steps are analogous to the corresponding steps described in WO 00/69810.

Step 2:

This reaction is analogous to similar reactions described in WO 00/69810.

Step 3:

If the product from step 2 is a benzoic acid ester, then the ester is hydrolysed (step 3). This step is similar to similar transformations described in WO 00/69810.

Steps 4 and 5:

Steps 4 & 5 are coupling of the benzoic acid with a β-alanine ester or R-isoserine ester followed by hydrolysis of the ester. These steps are similar to similar transformations described in WO 00/69810.

The general procedure (E) is further illustrated in the following example:

### Example 183

### General Procedure (E)

3-{4-[1-(4-Cyclohexylbenzyl)-3-(3,5-dichlorophenyl)ureido]benzoylamino}-2(R)-hydroxypropionic acid

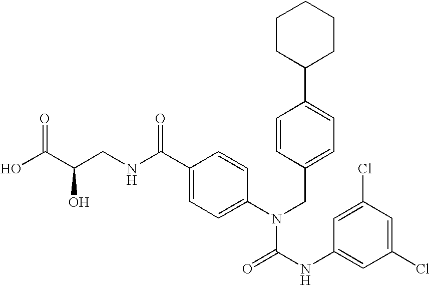

4-(4-Cyclohexylbenzylamino)benzoic acid methyl ester

4-Aminobenzoic acid methyl ester (500 mg, 3.31 mmol) was dissolved in methanol (25 ml) and 4-cyclohexylbenzaldehyd (1244 mg, 6.62 mmol) was added, resulting in immediately precipitation. Acetic acid (2.0 ml) and sodium cyano borohydride (600 mg; 8.7 mmol) was added, and the mixture was heated to reflux for 30 min. A clear solution was obtained. The mixture was cooled on an icebath, whereupon crystals appeared. The crystalline material was collected, washed with water and oven dried. Yield: 980 mg (91%).

HPLC-MS (Method (B)): m/z: 324 (M+1), 346 (M+Na), Rt: 5.54 min. ¹H-NMR(CDCl₃): δ 1.15-1.45 (m, 5H); 1.70-1.90 (m, 5H); 2.50 (m, 1H); 3.81 (s, 3H); 4.31 (s, 2H); 6.58 (d, 2H); 7.18 (d, 2H); 7.25 (d, 2H); 7.84 (d, 2H).

4-[1-(4-Cyclohexylbenzyl)-3-(3,5-dichlorophenyl)ureido]benzoic acid

4-(4-Cyclohexbenzylamino)benzoic acid methyl ester (4.19 g, 13.0 mmol) was slurried in acetonitrile (75 mL) and 3,5-dichlorophenylisocyanate (2.44 g, 13.0 mmol) was added. The mixture was refluxed for 6 hours under a nitrogen atmosphere. The mixture was cooled to room temperature, the precipitate filtered off, washed with cold acetonitrile and dried to afford 4.19 g (62%) of 4-[1-(4-cyclohexylbenzyl)-3-(3,5-dichlorophenyl)ureido]benzoic acid methyl ester. HPLC-MS (Method (B)): m/z: 581 (M+1), Rt: 6.60 min.

A solution of 4-[1-(4-cyclohexylbenzyl)-3-(3,5-dichlorophenyl)ureido]benzoic acid methyl ester (4.11 g, 1.27 mmol) in THF (20 mL) and methanol (20 mL) was stirred and 1 M NaOH was added (20 mL). The mixture was stirred for 30 minutes at room temperature and and then heated to 40° C. for 2 hours. The mixture was acidified (pH=2) with 1 M hydrochloric acid and the organic solvents was evaporated. The residue was partitioned between water and ethyl acetate and the organic phase was washed with water, dried and evaporated. The residue was recrystallised from acetonitrile to afford 1.07 g of 4-[1-(4-cyclohexylbenzyl)-3-(3,5-dichlorophenyl)ureido]benzoic acid. HPLC-MS (Method (B)): m/z: 497 (M+1), Rt: 6.02 min.

3-{4-[1-(4-Cyclohexylbenzyl)-3-(3,5-dichlorophenyl)ureido]benzoylamino}-2(R)-hydroxypropionic acid

A solution of 4-[1-(4-cyclohexylbenzyl)-3-(3,5-dichlorophenyl)ureido]benzoic acid (0.29 g; 0.59 mmol) in 1 mL of DMF and 4 mL of DCM was stirred while 1-hydroxybenzotriazole hydrate (0.095 g; 0.7 mmol) and EDAC (0.135 g; 0.7 mmol), were added. The mixture was stirred for 30 minutes at room temperature and R-isoserine methyl ester hydrochloride (0.12 g; 0.77 mmol) and DIPEA (0.30 mL; 1.73 mmol) were added. The mixture was stirred at room temperature for 16 hours and evaporated under reduced pressure. The residue was partitioned between water and ethyl acetate. The organic phase was separated, washed with water, dried (MgSO₄) and filtered through a bed of silica gel. The filtrate was evaporated and the intermediary ester was dissolved in a mixture of methanol (20 mL) and THF (20 mL) and 1 M sodium hydroxide (1.2 mL) was added. The mixture was stirred for 16 hours at room temperature and concentrated to an oil. The pH was adjusted to 3 by addition of 1 M hydrochloric acid and the solution was concentrated to an oil. The oil was purified by chromatography to afford 0.13 g (38%) of the title compound.

HPLC-MS (Method (B)): m/z: 584 (M+1), Rt: 5.57 min. ¹H-NMR(DMSO-d₆): δ 1.00-1.70 (m, 10H); 2.42 (m, 1H), 3.46 (m, 2H), 4.16 (t, 1H), 4.93 (s, 2H), 7.15 (m, 5H), 7.34 (d, 2H), 7.59 (d, 2H), 7.85 (d, 1H), 8.48 (t, 1H), 8.77 (s, 1H).

The following examples were made as described above.

### Example 184

### General Procedure (E)

3-{4-[1-(4-Cyclohexyl-benzyl)-3-(3,5-dichloro-phenyl)ureido]benzoylamino}propionic acid

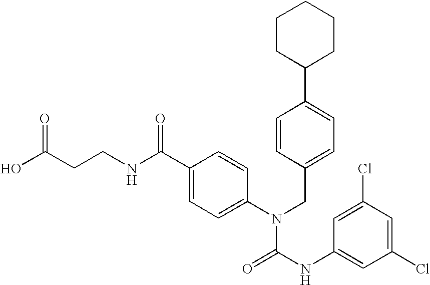

HPLC-MS (Method (B)): m/z: 568 (M+1), Rt: 5.78 min

### Example 185

### General Procedure (E)

3-{4-[1-(4-Cyclohex-1-enyl-benzyl)-3-(3,5-dichlorophenyl)ureido]benzoylamino}propionic acid

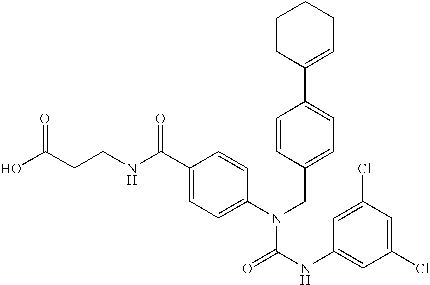

HPLC-MS (Method (B)): m/z: 566 (M+1), Rt: 5.42 min

General Procedure F:

General procedure (F) may be used for solid phase preparation of compounds of general formula (Id):

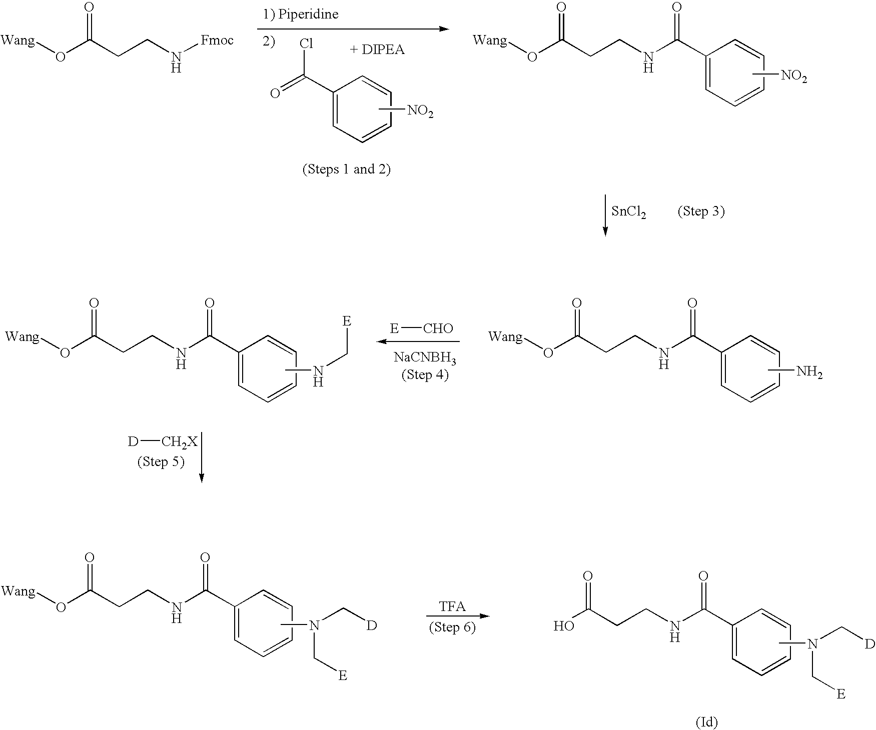

wherein E and D independently are aryl or heteroaryl and are both optionally substituted as defined previously.

Steps 1 to 2:

These steps are analogous to the corresponding steps described in WO 00/69810 and WO 02/00612.

Step 3:

The reduction of aromatic nitro groups on solid phase is generally known (F. Z. Dörwald, “Organic Synthesis on Solid Phase”, 1^{st} Edition Wiley-VCH: Weinheim, 2000, p. 246-247), and is performed using an excess of tin (II) chloride dihydrate in a polar organic solvent such as DMF or NMP. The reaction is performed at 20-100° C., preferable at ambient or slightly elevated temperature.

Step 4:

This reaction is generally known (F. Z. Dörwald, “Organic Synthesis on Solid Phase”, 1^{st} Edition Wiley-VCH: Weinheim, 2000, p. 239-241), and is achieved by using an excess of aldehyde, sodium cyano borohydride and a proton source such as acetic acid. The reaction is performed at 20-100° C. preferable at 40-80° C. in a polar organic solvent such as DMF or NMP.

Steps 5:

This reaction is generally known (A. P. Shawcross & S. P. Stanforth, Tetrahedron, 45(22), 1989, 7063-7076) and is performed by adding alkyl halides to anilines in the presence of base. Optionally, sodium iodide can be added to improve yield and reaction rate. The reaction is performed in organic solvents, preferably polar organic solvents such as DMF, tetrahydrofurane or DMSO, at 20-100° C. preferable at 40-80° C.

Step 6: Cleavage from Resin

This step is analogous to the corresponding transformations described in WO 00/69810 and WO 02/00612.

The general procedure (F) is further illustrated in the following example:

### Example 186

### General Procedure (F)

3-{4-[(9H-Fluoren-2-ylmethyl)-(4-trifluoromethoxybenzyl)amino]benzoylamino}propionic acid

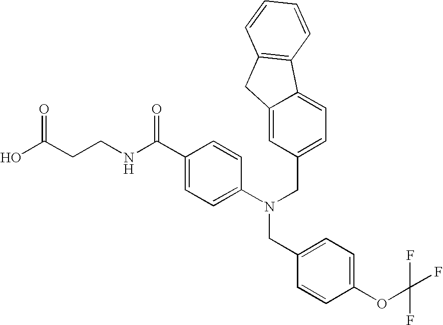

Step 1: Fmoc-β-Ala-Wang resin (5.0 g, 0.31 mmol/g, 1.55 mmol) was treated with piperidine (20% in NMP, 20 ml) for 30 min and the resin was drained. This was repeated once. The resin was then washed with DMF (5×).

Step 2: A solution of DIPEA (3 ml) in NMP (17 ml) was added, followed by slow addition of a solution of p-nitrobenzoylchloride (2.88 g; 15.5 mmol, 10 eq.) in NMP (20 ml). The mixture was shaken for 3 h, then drained. The resin was washed with DMF (5×).

Step 3: A solution of SnCl₂. 2H₂O (10.5 g; 46.5 mmol, 30 eq.) in NMP (30 ml) was added to the resin. The mixture was shaken at room temperature for 16 h. The resin was drained and washed with DMF (3×) and DCM (10×), then dried under vacuum for 16 h. Yield: 5.20 g.

Step 4: Dry resin (100 mg; 54 mmol; 0.54 mmol/g) prepared as described above, was swelled in DCM for 30 min. Fluorene-2-carboxaldehyde (194.5 mg; 1 mmol) dissolved in DMF (1 ml) was added followed by a solution of sodium cyano borohydride (138 mg; 2 mmol) in DMF—acetic acid (1.2 ml, 5:1). The reaction mixture was heated to 80° C. over night. The resin was drained for solvent and reactants, and subsequently washed with MeOH (3×), DMF (5×) and DCM (4×) to give resin bound 3-{4-[(9H-fluoren-2-ylmethyl)amino]benzoylamino}propionic acid.

Step 5: Resin bound 3-{4-[(9H-fluoren-2-ylmethyl)amino]benzoylamino}propionic acid (50 mg; 27 mmol; 0.54 mmol/g) was washed twice with propionitril. A solution of 4-trifluoromethoxybenzyl bromide (76.5 mg; 0.3 mmol) in propionitril (400 ul) was added, followed by a solution of tetrabutylammonium iodide (111 mg, 0.3 mmol) in propionitril (500 ul). Finally DIPEA (55 ul, 0.3 mmol) was added, and the reaction mixture was shaken at 60° C. over night. The solvent was removed, and the resin was washed 10 times with dichloromethane.

Step 6: The product was cleaved from the resin using 50% TFA in DCM. Solvent was removed by speed evacuation to give 10-12 mg of pure title material.

HPLC-MS (Method (B)): m/z: 561 (M+1), Rt: 5.11 min.

In a similar manner, the following compounds were made:

### Example 187

### General Procedure (F)

3-{4-[Benzyl-(4-cyclohexylbenzyl)amino]benzoylamino}propionic acid

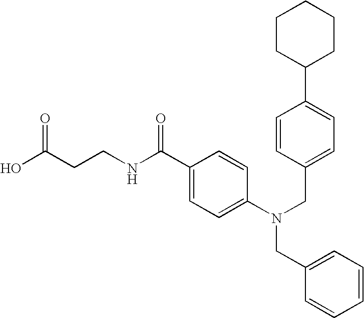

HPLC-MS (Method (B)): m/z: 471 (M+1), Rt: 6.31 min.

### Example 188

### General Procedure (F)

3-{(4-(4-Cyclohexylbenzyl)-[2-oxo-2-(4-trifluoromethylphenyl)ethyl]amino}benzoylamino)-propionic acid

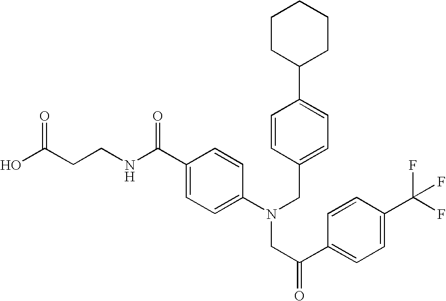

HPLC-MS (Method (B)): m/z: 567 (M+1), Rt: 6.41 min.

### Example 189

### General Procedure (F)

3-{4-[Bis(4-trifluoromethoxybenzyl)amino]benzoylamino}propionic acid

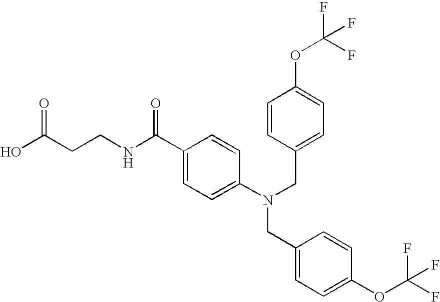

HPLC-MS (Method (B)): m/z: 557 (M+1), Rt: 5.82 min.

### Example 190

### General Procedure (F)

3-{4-[Indan-5-ylmethyl-(4-trifluoromethoxybenzyl)amino]benzoylamino}propionic acid

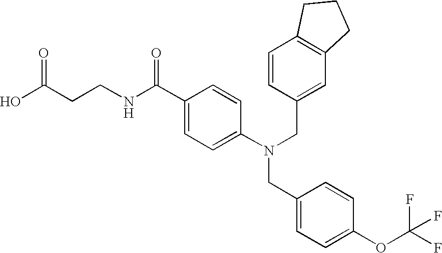

HPLC-MS (Method (B)): m/z: 513 (M+1), Rt: 5.86 min.

### Example 191

### General Procedure (F)

3-{4-[(4-Bromo-2-fluorobenzyl)-(4-trifluoromethoxybenzyl)amino]benzoylamino}propionic acid

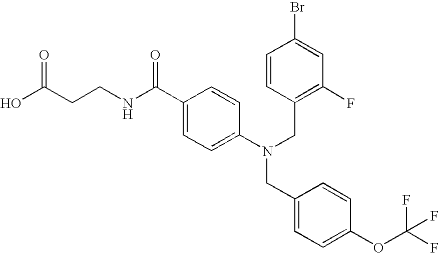

### Example 192

### General Procedure (F) 3-{4-[(4-Chlorobenzyl)-(4-trifluoromethoxybenzyl)amino]benzoylamino}propionic acid

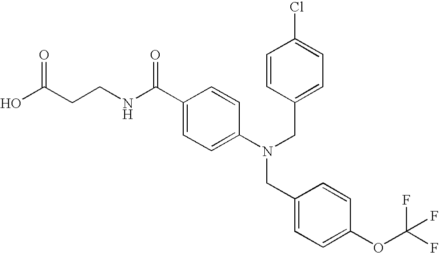

### Example 193

### General procedure (F)

3-{4-[(4-Bromobenzyl)-(4-isopropylbenzyl)amino]benzoylamino}propionic acid

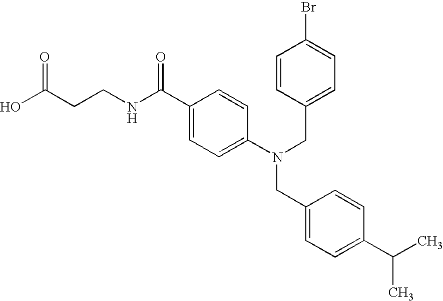

### Example 194

### General Procedure (F)

3-[4-(Bis(biphenyl-4-ylmethyl)amino)benzoylamino]propionic acid

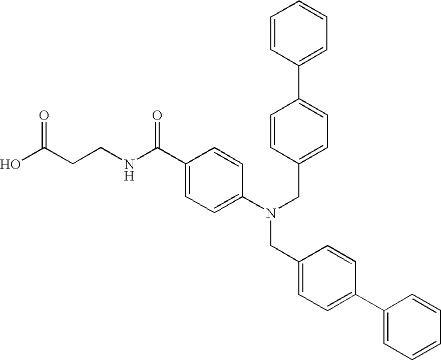

### Example 195

### General Procedure (F)

3-{4-[Biphenyl-4-ylmethyl-(4-trifluoromethoxybenzyl)amino]benzoylamino}propionic acid

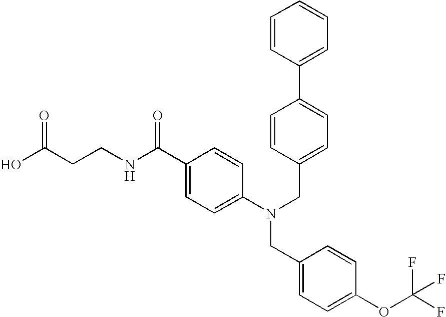

### Example 196

### General Procedure (F)

3-{4-[Bis(4-trifluoromethylbenzyl)amino]benzoylamino}propionic acid

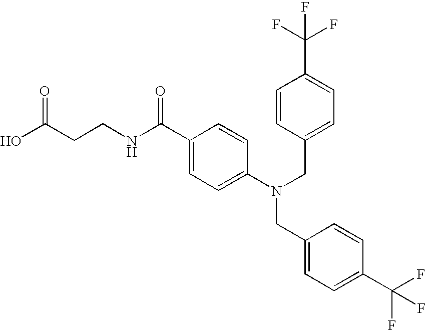

### Example 197

### General Procedure (F)

3-(4-{(4-Bromobenzyl)-[3-(3,5-dichlorophenoxy)benzyl]amino}benzoylamino)propionic acid

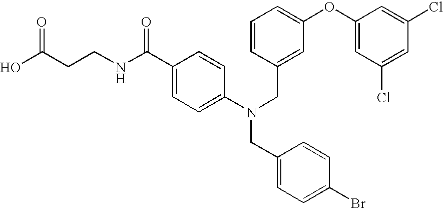

### Example 198

### General Procedure (F)

3-(4-{(3-Chlorobenzyl)-[3-(3,5-dichlorophenoxy)benzyl]amino}benzoylamino)propionic acid

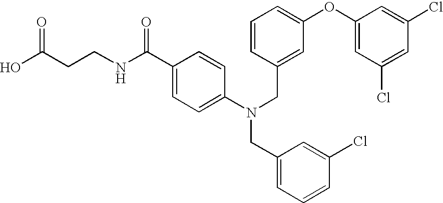

### Example 199

### General Procedure (F)

3-{4-[(3-Bromo-4-fluorobenzyl)-(4-trifluoromethylbenzyl)amino]benzoylamino}propionic acid

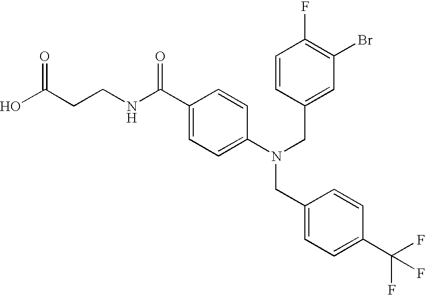

### Example 200

### General Procedure (F)

3-{4-[Bis(4-tert-butylbenzyl)amino]benzoylamino}propionic acid

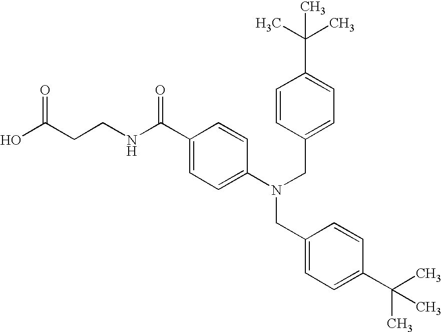

### Example 201

### General Procedure (F)

3-{4-[(4-Bromobenzyl)-(4-isobutylbenzyl)amino]benzoylamino}propionic acid

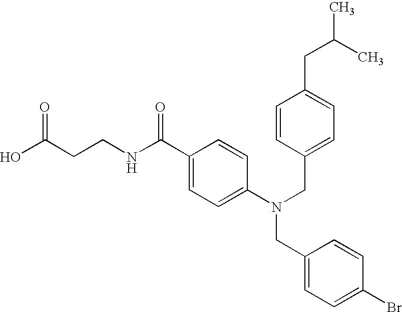

### Example 202

### General Procedure (F)

3-{4-[(4-Isobutylbenzyl)-(4-trifluoromethylbenzyl)amino]benzoylamino}propionic acid

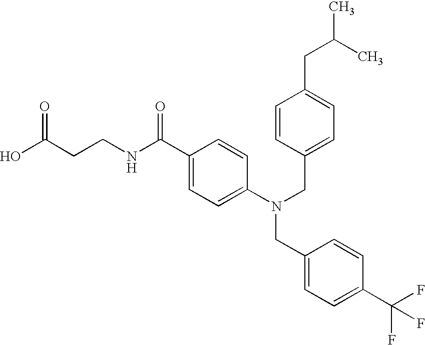

### Example 203

### General Procedure (F)

3-{4-[(3,5-Dichlorobenzyl)-(3-trifluoromethoxybenzyl)amino]benzoylamino}propionic acid

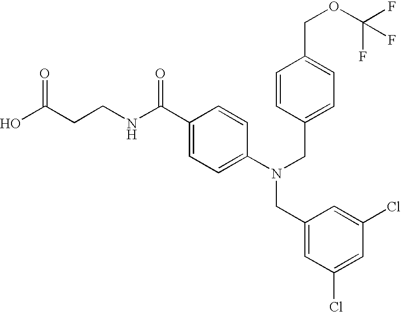

### Example 204

### General Procedure (F)

3-{4-[(4-Cyclohexylbenzyl)-(4-[1,2,3]thiadiazol-4-ylbenzyl)amino]benzoylamino}propionic acid

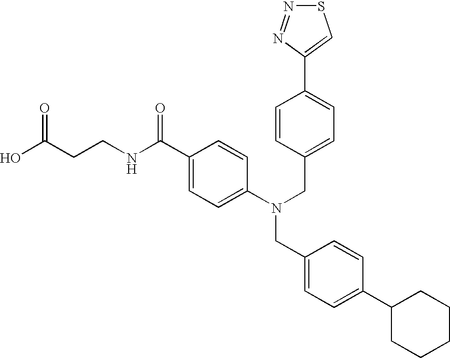

General Procedure G:

General procedure (G) may be used for solution phase preparation of compounds of general formula (Id):

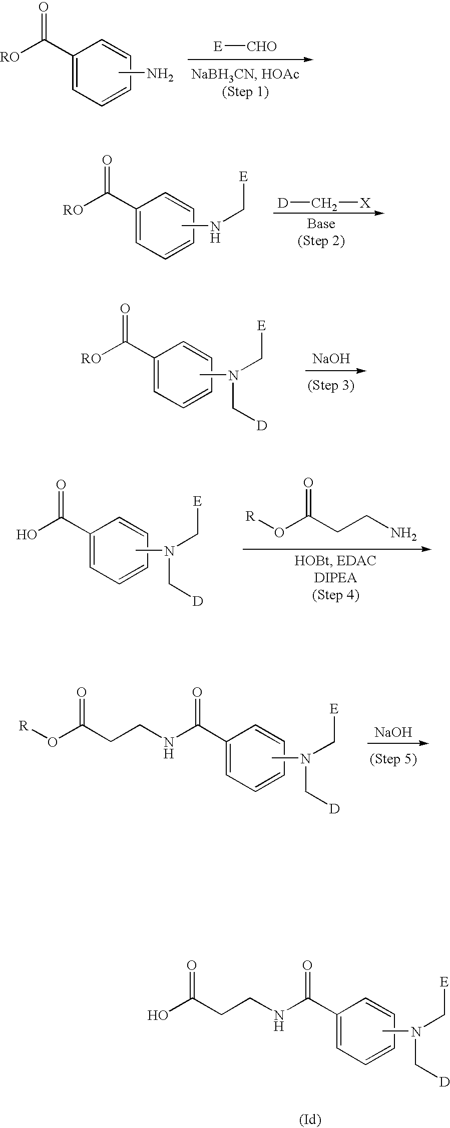

wherein R are C₁₋₆-alkyl, and E and D independently are aryl or heteroaryl and are both optionally substituted as defined previously.

Step 1:

The reductive amination steps are analogous to the corresponding steps described in WO 00/69810.

Step 2:

This reaction is generally known (A. P. Shawcross & S. P. Stanforth, Tetrahedron, 45(22), 1989, 7063-7076) and is performed by adding alkyl halides to anilines in the presence of base.

Step 3:

This step is an ester hydrolysis similar to similar transformations described in WO 00/69810.

Steps 4 and 5:

Steps 4 & 5 are coupling of the derivatized benzoic acid with a β-alanine ester followed by hydrolysis of the ester. These steps are similar to similar transformations described in WO 00/69810.

The general procedure (G) is further illustrated in the following example:

### Example 205

### General Procedure (G)

3-{4-[(4-Cyclohexylbenzyl)-(4-trifluoromethoxybenzyl)amino]benzoylamino}propionic acid

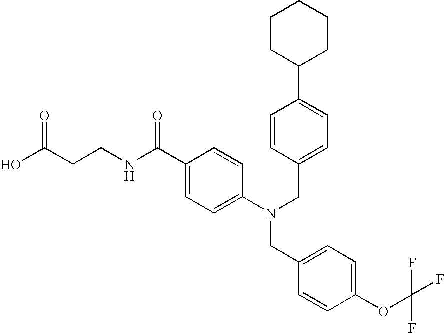

4(4-Cyclohexylbenzylamino)benzoic acid methyl ester

4-Aminobenzoic acid methyl ester (500 mg, 3.31 mmol) was dissolved in methanol (25 ml) and 4-cyclohexylbenzaldehyd (1244 mg, 6.62 mmol) was added, resulting in immediately precipitation. Acetic acid (2.0 ml) and sodium cyano borohydride (600 mg; 8.7 mmol) was added, and the mixture was heated to reflux for 30 min. A clear solution was obtained. The mixture was cooled on an icebath, whereupon crystals appeared. The crystalline material was collected, washed with water and oven dried. Yield: 980 mg (91%).

HPLC-MS (Method (B)): m/z: 324 (M+1), 346 (M+Na), Rt: 5.54 min. ¹H-NMR(CDCl₃): δ 1.15-1.45 (m, 5H); 1.70-1.90 (m, 5H); 2.50 (m, 1H); 3.81 (s, 3H); 4.31 (s, 2H); 6.58 (d, 2H); 7.18 (d, 2H); 7.25 (d, 2H); 7.84 (d, 2H).

4[(4-Cyclohexylbenzyl)-(4-trifluoromethoxybenzyl)amino]benzoic acid methyl ester

4-(4-Cyclohexylbenzylamino)benzoic acid methyl ester (500 mg 1.54 mmol) was dissolved in DMF (4 ml). To the clear solution was added potassium carbonate (650 mg; 4.71 mmol) followed by 4-trifluoromethoxybenzylbromide (627 mg, 6.0 mmol). The mixture was heated to 90° C. for 3 h, then cooled and partitioned between ethyl acetate (10 ml) and water (10 ml). The organic phase was separated, washed once with brine and dried with anhydrous sodium sulphate. Solvent was removed by rotary evaporation. The residual oil was purified by column chromatography, using 2% ethyl acetate-petrol ether as eluent. Pure fractions (R_{f}=0.8 in 10% ethyl acetate-petrol ether) were pooled and taken to dryness, to give title material as a colourless oil. Yield: 617 mg (80%). HPLC-MS (Method (B)): m/z: 498 (M+1), Rt: 6.46 min. ¹H-NMR(CDCl₃): δ 1.15-1.45 (m, 5H); 1.70-1.90 (m, 5H); 2.51 (m, 1H); 3.79 (s, 3H); 4.62 (s, 2H); 4.65 (s, 2H); 6.68 (d, 2H); 7.15 (d, 2H); 7.21 (d, 2H); 7.22 (d, 2H); 7.40 (d, 2H); 7.80 (d, 2H).

4[(4-Cyclohexylbenzyl)-(4-trifluoromethoxybenzyl)amino]benzoic acid

4-[(4-Cyclohexylbenzyl)-(4-trifluoromethoxybenzyl)amino]benzoic acid methyl ester (610 mg; 1.22 mmol) was dissolved in ethanol (10 ml). 4N sodium hydroxide solution (2 ml), was added, and the solution was heated to reflux for 1 h. The solution was cooled on an ice bath, and acetic acid (3 ml) was added. The precipitate which formed was collected by filtration, washed with water and oven dried, to give 480 mg (81%) of pure title material as white powder. HPLC-MS (Method (B)): m/z: 484 (M+1), Rt: 6.07 min.

¹H-NMR(CDCl₃): δ 1.15-1.45 (m, 5H); 1.70-1.90 (m, 5H); 2.45 (m, 1H); 4.63 (s, 2H); 4.67 (s, 2H); 6.69 (d, 2H); 7.09 (d, 2H); 7.15 (d, 2H); 7.19 (d, 2H); 7.21 (d, 2H); 7.89 (d, 2H).

3-{4-[(4-Cyclohexylbenzyl)-(4-trifluoromethoxybenzyl)amino] benzoylamino}propionic acid ethyl ester

4-[(4-Cyclohexylbenzyl)-(4-trifluoromethoxybenzyl)amino]benzoic acid (300 mg; 0.62 mmol) was dissolved in a mixture of DCM (2.0 ml) and DMF (1.0 ml). Ethyl dimethylaminopropyl carbodiimid hydrochloride (144 mg; 0.75 mmol) and 1-hydroxybenzotriazol (113 mg; 0.75 mmol) were added. The mixture was stirred at ambient temperature for 2 h, then α-alanine ethyl ester hydrochloride (142 mg; 0.93 mmol) and DIPEA 318 ul, 1.86 mmol) were added. The mixture was left stirring at room temperature over night, then diluted with DCM (25 ml) and washed twice with water and once with brine. The organic solution was then dried with anhydrous sodium sulphate and taken to dryness by rotary evaporation, to give 310 mg (86%) of clear oil. HPLC-MS (Method (B)): m/z: 583 (M+1), Rt: 6.13 min.

¹H-NMR(CDCl₃): δ 1.19 (t, 3H); 1.25-1.45 (m, 5H); 1.60-1.90 (m, 5H); 2.49 (m, 1H); 2.54 (t, 2H); 3.65 (q, 2H); 4.12 (q, 2H); 4.61 (s, 2H); 4.65 (s, 2H); 6.62 (t, 1H); 6.69 (d, 2H); 7.09 (d, 2H); 7.15 (d, 2H); 7.19 (d, 2H); 7.21 (d, 2H); 7.60 (d, 2H).

3-{4-[(4-Cyclohexylbenzyl)-(4-trifluoromethoxybenzyl)amino]benzoylamino}propionic acid

3-{4-[(4-Cyclohexylbenzyl)-(4-trifluoromethoxybenzyl)amino]benzoylamino}propionic acid ethyl ester (310 mg; 0.53 mmol) was dissolved in ethanol (6 ml). 4N sodium hydroxide solution (3 ml), was added, and the solution was stirred at room temperature for 30 min. Then acetic acid (2.0 ml) was added, and the reaction mixture partitioned between water (30 ml) and DCM (30 ml). The organic phase was washed once with water and once with brine, then dried over anhydrous sodium sulphate. The solvent was removed, and the residue evaporated twice from acetonitril to remove water and solvent traces. The compound, which remained as an oil, was then evaporated once from DCM, whereupon a white foam was obtained. Yield: 180 mg (60%) of the title compound. HPLC-MS (Method (B)): m/z: 555 (M+1), Rt: 5.63 min.

¹H-NMR(CDCl₃): δ 1.10-1.45 (m, 5H); 1.65-1.90 (m, 5H); 2.46 (m, 1H); 2.50 (m, 2H); 3.54 (m, 2H); 4.55 (s, 2H); 4.57 (s, 2H); 6.54 (d, 2H); 6.70 (t, 1H); 7.03 (d, 2H); 7.08-7.15 (m, 8H); 7.52 (d, 2H).

General Procedure H:

Alternatively, general procedure (H) may be used for solution phase preparation of compounds of general formula (Id):

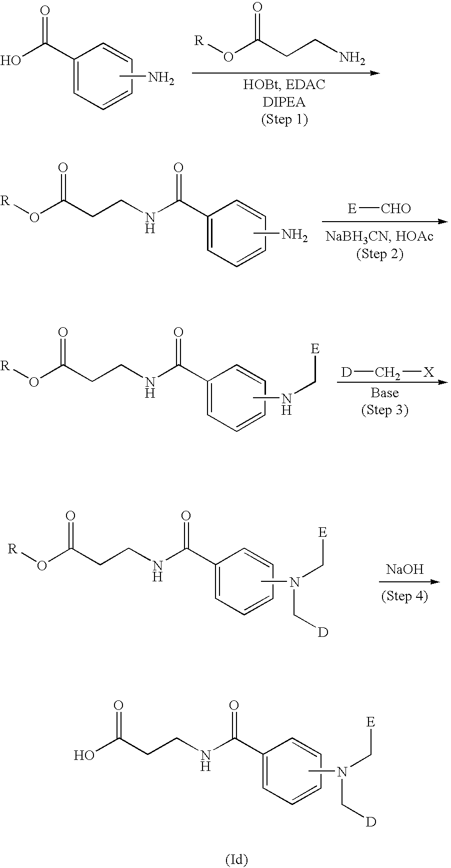

wherein R are C₁₋₆-alkyl, and E and D independently are aryl or heteroaryl and are both optionally substituted as defined previously.

Step 1:

This is a coupling reaction between an amino benzoic acid and an β-alanine ester. The step is similar to transformations described in WO 00/69810

Step 2:

The reductive amination steps are analogous to the corresponding steps described in WO 00/69810.

Step 3:

This reaction is generally known (A. P. Shawcross & S. P. Stanforth, Tetrahedron, 45(22), 1989, 7063-7076) and is performed by adding alkyl halides to anilines in the presence of base.

Steps 4:

Steps 4 is an ester hydrolysis similar to those described in WO 00/69810.

The general procedure (H) is illustrated in the following example:

### Example 206

### General Procedure (H)

3-{4-[(9H-Fluoren-2-ylmethyl)-(4-trifluoromethoxybenzyl)amino]benzoylamino}propionic acid

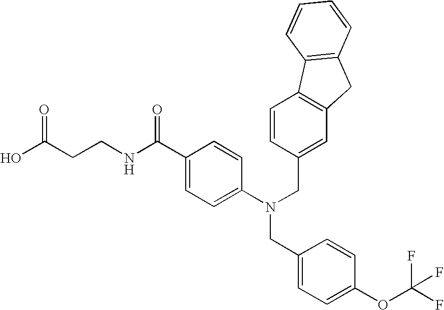

3-(4-Aminobenzoylamino)propionic acid ethyl ester

4-Aminobenzoic acid (4.50 g; 32.5 mmol) and 1-hydroxybenzotriazole hydrate (5.50 g; 36.0 mmol) was dissolved in THF (100 ml). 1-Ethyl-3-(3′-dimethylaminopropyl)carbodiimide hydrochloride (6.80 g; 36 mmol) was added. A semi crystalline solid appeared. Dichloromethane (50 ml) was added and a clear solution was obtained. The mixture was stirred for an additional 10 min., then ethyl 3-aminopropanoic acid hydrochloride (5.00 g; 32.5 mmol) and diisopropylethylamine (17.0 ml; 97.5 mmol) were added. The mixture was stirred for 2 h at ambient temperature, and then heated to reflux for 10 min. The reaction mixture was allowed to cool, then partitioned between water (100 ml) and DCM (100 ml). The water phase was extracted trice with DCM (3×100 ml). The combined organic phases were then dried over anhydrous sodium sulphate, filtered and evaporated to dryness by rotary evaporation in vacuo. A quantitative yield of amber coloured oil was obtained, which crystallized upon standing. HPLC-MS (Method (B)): m/z: 237 (M+1), Rt: 1.44 min. ¹H-NMR(DMSO-d₆): δ 1.15 ppm (t, 3H); 2.50 (t,2H, collapse with the DMSO-d₅ signal); 3.55 (q, 2H); 4.02 (q, 2H); 6.51 (d, 2H); 7.53 (d, 2H); 8.04 (t, 1H).

3-{4[(9H-Fluoren-2-ylmethyl)amino] benzoylamino}propionic acid ethyl ester

3-(4-Aminobenzoylamino)propionic acid ethyl ester (12.0 g; 50.8 mmol) was dissolved in ethanol (200 ml). Then a solution of fluorene-2-carboxaldehyde (9.86 g; 50.8 mmol) in ethanol (100 ml) was added to give a clear yellow solution. The solution was heated for reflux for 10 min. then cooled to room temperature. Acetic acid (30 ml) and solid sodium cyano borohydride (3.5 g; 50.7 mmol) were added. The mixture was refluxed for 30 min; then slowly allowed to cool to room temperature. The title material, which slowly crystallised out of solution, was collected and washed with water. The crystals were then dried in an oven over night. Yield: 17.46 g (83%). HPLC-MS (Method (B)): m/z: 415 (M+1), Rt: 4.37 min. ¹H-NMR(CDCl₃) δ 1.25 ppm (t, 3H); 2.60 (t, 2H); 3.69 (q, 2H); 3.88 (s, 2H); 4.15 (q, 2H); 4.44 (s, 2H); 6.62 (d, 2H); 7.28-7.40 (m, 3H); 7.54 (d, 2H); 7.62 (d, 2H); 7.76 (t, 2H).

3-{4-[(9H-Fluoren-2-ylmethyl)-(4-trifluoromethoxybenzyl)amino]benzoylamino}-propionic acid

A mixture of 3-{4-[(9H-fluoren-2-ylmethyl)amino]benzoylamino}propionic acid ethyl ester (500 mg; 1.2 mmol), 4-trifluoromethoxybenzyl bromide (461 mg; 1.8 mmol), potassium carbonate (358 mg; 3.6 mmol) and sodium iodide (685 mg; 3.6 mmol) in acetonitril (6 ml) was heated to 60° C. for 16 h. The reaction mixture was then cooled to room temperature and partitioned between water and ethyl acetate. The organic solution was collected, concentrated in vacuo, to give an oil. The oil was flushed through a short pad of silica and the material with R_{f}=0.8 (in ethyl acetate-ethanol-petrol ether (45:10:45)) was collected and concentrated to give the ethyl ester derivative of the title material. Yield: 500 mg (70%). HPLC-MS (Method (B)): m/z: 589 (M+1), Rt: 5.65 min. The ethyl ester (500 mg; 0.8 mmol) was then dissolved in ethanol (6 ml), and 4N aqueous sodium hydroxide solution (3 ml) was added. The mixture was stirred at room temperature for 1 h. Acetic acid (3.0 ml) was added, and the reaction mixture was partitioned between water and DCM. The organic layer was then collected, washed with brine and concentrated to give an oil. The oil was flushed through a short pad of silica using an eluent of ethyl acetate-acetic acid-petrol ether (45:10:45), to give 394 mg (82%) of pure title compound. HPLC-MS (Method (B)): m/z: 561 (M+1), Rt: 5.11 min. ¹H-NMR(CDCl₃) δ 2.66 ppm (t, 2H); 3.65 (q, 2H); 3.84 (s, 2H); 4.68 (s, 2H); 4.72 (s, 2H); 6.62 (t,1H); 6.72 (d, 2H); 7.08-7.40 (m, 8H); 7.51 (d, 1H); 7.60 (d, 2H); 7.73 (t, 2H).

In a similar manner the following compounds were made:

### Example 207

### General Procedure (H)

3-{4-[(9H-Fluoren-2-ylmethyl)-(4-trifluoromethylbenzyl)amino]benzoylamino}propionic acid

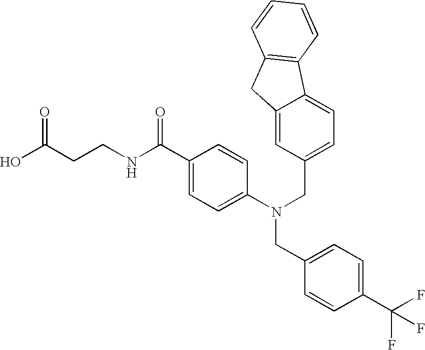

HPLC-MS (Method (B)): m/z: 545 (M+1), Rt: 5.02 min. ¹H-NMR(CDCl₃) δ 2.68 (t, 2H); 3.66 (q, 2H); 3.85 (s, 2H); 4.75 (s, 4H, the two benzylic methylene groups collapses); 6.60 (t, 1H); 6.72 (d, 2H); 7.14-7.40 (m, 7H); 7.50-7.65 (m, 4H); 7.73 (t, 2H).

### Example 208

### General Procedure (H)

3-{4-[(4-tert-Butylbenzyl)-(9H-fluoren-2-ylmethyl)amino]benzoylamino}propionic acid

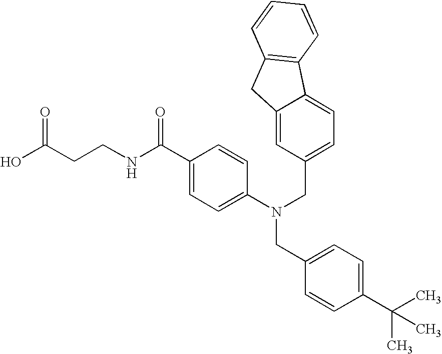

HPLC-MS (Method (B)): m/z: 533 (M+1), Rt: 5.51 min. ¹H-NMR(CDCl₃) δ 1.30 (s, 9H); 2.65 (t, 2H); 3.68 (q, 2H); 3.85 (s, 2H); 4.69 (s, 2H); 4.73 (s, 2H); 6.57 (t, 1H); 6.75 (d, 2H); 7.12 (d, 2H); 7.19 (d, 1H); 7.28 (d, 1H); 7.30-7.40 (m, 4H); 7.52 (d, 1H); 7.61 (d, 2H); 7.73 (t, 2H).

### Example 209

### General Procedure (H)

3-{4-[(4-tert-Butylbenzyl)-(4-cyclohexylbenzyl)amino]benzoylamino}propionic acid

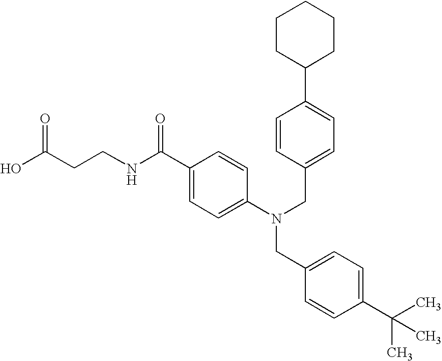

HPLC-MS (Method (B)): m/z: 527 (M+1), Rt: 6.00 min. ¹H-NMR(CDCl₃) δ 1.28 (s, 9H); 1.30-1.45 (m, 5H); 1.70-1.95 (m, 5H); 2.47 (m, 1H); 2.67 (t, 2H); 3.68 (q, 2H); 4.63 (s, 4H, the two benzylic methylene groups collapses); 6.56 (t, 1H); 6.71 (d, 2H); 7.08-7.20 (m, 6H); 7.32 (d, 2H); 7.55 (d, 2H).

### Example 210

### General Procedure (H)

3-{4-[(4-Cyclohexylbenzyl)-(4-trifluoromethylbenzyl)amino]benzoylamino}propionic acid

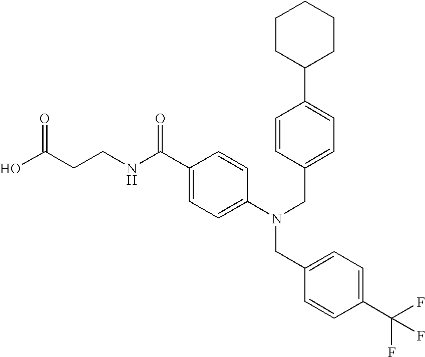

HPLC-MS (Method (B)): m/z: 539 (M+1), Rt: 5.51 min. ¹H-NMR(CDCl₃) δ 1.15-1.50 (m, 5H); 1.68-1.92 (m, 5H); 2.48 (m, 1H); 2.68 (t, 2H); 3.68 (q, 2H); 4.65 (s, 2H); 4.71 (s, 2H); 6.60 (t, 1H); 6.68 (d, 2H); 7.10 (d, 2H); 7.16 (d, 2H); 7.31 (d, 2H); 7.56 (d, 2H); 7.60 (d, 2H).

General Procedure I:

General procedure (I) may be used for the solution phase of compounds of general formula (Ie):

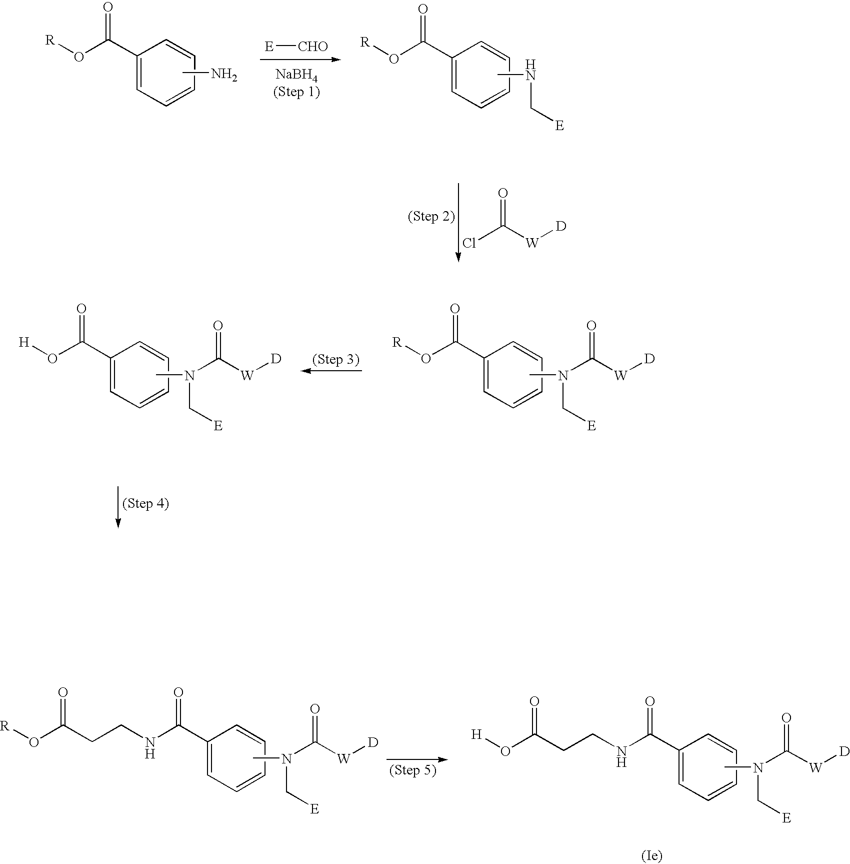

wherein

R are hydrogen or C₁₋₆-alkyl,

E and D independently are aryl or heteroaryl and are both optionally substituted as defined above, and W are C═C, CH₂

Steps 1:

The reductive amination steps are analogous to the corresponding steps described in WO 00/69810.

Step 2:

This reaction is known (see Albright, J., D. et al., J. Med. Chem. 1983, 26 (10), 1378-1393) and is performed by reacting carbonyl chlorides with arylamines in the presence of base. As base, an organic amine such as triethylamine, pyridine, DIPEA or 1,4-diazabicyclo[2.2.2.]octane (DABCO) can be used. The reaction is performed at 0-80° C., preferably at ambient temperature in an inert aprotic polar solvent such as DCM, DMF, THF or NMP.

Step 3:

If the product from step 2 is a benzoic acid ester, then the ester is hydrolysed (step 3). This step is similar to similar transformations described in WO 00/69810.

Steps 4 and 5:

Steps 4 & 5 are coupling of the benzoic acid with a β-alanine ester followed by hydrolysis of the ester. These steps are similar to similar transformations described in WO 00/69810.

The general procedure (I) is further illustrated in the following example:

### Example 211

### General Procedure (I)

3-(4-{(4-Cyclohex-1-enylbenzyl)-[3-(3-trifluoromethoxyphenyl)acryloyl]amino}benzoylamino) -propionic acid

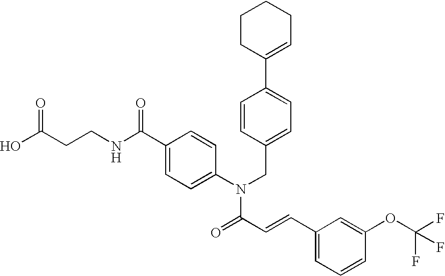

4-(4-Cyclohex-1-enylbenzylamino)benzoic acid methyl ester

4-Aminobenzoic acid methyl ester (2.40 g, 16.1 mmol) was warmed in methanol (20 mL) until dissolution and a solution of 4-cyclohex-1-enylbenzaldehyde (3.0 g, 16.1 mmol) in methanol (10 mL) was added. The mixture was stirred at room temperature and a catalytic amount of p-toluenesulphonic acid was added. The mixture was cooled in an ice bath and stirring was continued for 2 hours. The precipitate was filtered off, washed with cold methanol, dried and dissolved in a mixture of methanol (20 mL) and NMP (20 mL). The mixture was stirred and NaBH₄ (1.14 g, 30.9 mmol) was added in portions during 30 minutes. The mixture was stirred for 2 hours at room temperature and partitioned between water and ethyl acetate. The aqueous phase was further extracted with ethyl acetate. The combined organic extracts was washed with water, dried (MgSO₄), filtered through a bed of silica gel and evaporated. The residue was triturated with petroleum ether, isolated, washed with petroleum ether and dried to afford 3.01 g (58%) of 4-(4-cyclohex-1-enylbenzylamino)benzoic acid methyl ester HPLC-MS (Method (B)): m/z: 344 (M+23), Rt: 5.70 min. ¹H-NMR(DMSO-d₆): δ 1.60 (m, 4H), 1.69 (m, 4H), 2.14 (m, 4H), 2.33 (m, 4H), 3.72 (s, 3H), 4.31(d, 2H), 6.11 (t, 1H), 6.60 (d, 2H), 7.10 (t, 1H), 7.27 (d, 2H), 7.35 (d, 2H), 7.66 (d, 2H).

The aldehyde starting material was prepared as shown below.

4-Cyclohex-1-enylbenzaldehyde

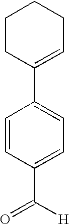

Magnesium turnings (14.6 g, 600 mmol) was placed in a dry 4-necked flask. Dry THF (50 mL) and a crystal of iodine were added. A mixture of 2-(4-bromophenyl)-[1,3]-dioxolane (Tetrahedron, 57, No.28, (2001), 5991-6002) (135 g, 589 mmol) in dry THF (200 mL) was slowly added to initiate the reaction. After the reaction had started, the addition of 2-(4-bromophenyl)-[1,3]-dioxolane was continued at such a rate that the temperature was maintained between 35 and 40° C. After the addition was complete the mixture was stirred for 2 hours and then cooled to 5° C. on an ice bath. Cyclohexanone (57.8 g, 580 mmol) was added dropwise while maintaining the temperature below 10° C. The mixture was stirred for 18 hours at room temperature and two third of the THF was removed in vacuo. The residue was poured into a mixture of ammonium chloride (65 g) in ice water (1 liter) and extracted with ethyl acetate. The organic phase was washed with water, dried (magnesium sulphate), filtered and evaporated in vacuo. The residual oil was slurred in petroleum ether to afford 48 g of 1-(4-[1,3]dioxolan-2-yl-phenyl)cyclohexanol as a solid.

HPLC-MS (Method A): m/z=231 (M+1); Rt=3.27 min.

1-(4-[1,3]Dioxolan-2-yl-phenyl)cyclohexanol (45 g) and p-toluenesulfonic acid (3.4 g) in 300 mL of toluene were refluxed for 3 hours under Dean-Stark conditions. After cooling, ethyl acetate and a saturated sodium hydrogen carbonate solution were added. The organic layer was washed twice with water, dried (magnesium sulphate), filtered and concentrated in vacuo. The residual oil was dissolved in glacial acetic acid (250 mL) and 1 M hydrochloric acid (25 mL) was added and the mixture was stirred at 50° C. for 2 hours. After cooling, the mixture was concentrated in vacuo. The residual oil was partitioned between ethyl acetate and water. The organic phase was washed three times with water, dried (magnesium sulphate), filtered and concentrated in vacuo. The residual oil was distilled in vacuo and the fraction boiling at 120-130° C. (0.2 mmHg) was collected to afford 4.7 g of the title compound.

¹H NMR (CDCl₃): δ 1.72 (m, 4H), 2.25 (m, 2H), 2.43 (m, 2H), 6.30 (m, 1H), 7.53 (d, 2H), 7.82 (d, 2H), 9.98 (s, 1H).

4-{(4-Cyclohex-1-enylbenzyl)-[3-(3-trifluoromethoxyphenyl)acryloyl]amino}benzoic acid methy ester

A mixture of 4-(4-cyclohex-1-enylbenzylamino)benzoic acid methyl ester (0.5 g, 1.55 mmol) and triethylamine (0.6 mL, 4.67 mmol) in dry DCM (15 mL) was stirred in an ice bath while 3-(3-trifluoromethoxyphenyl)acryloyl chloride (0.58 g, 2.33 mmol) (prepared from the corresponding acid by reaction with thionyl chloride in toluene) was added. The ice bath was removed and stirring was maintained for 16 hours. The mixture was evaporated and the oily residue was partitioned between ether and water. The organic phase was washed five times with water, filtered through a bed of silica gel and evaporated to an oil. The oil was subject to column chromatography (silica gel) with toluene:DCM (8:2) as eluent. The proper fractions was collected and evaporated to afford 0.68 g (82%) of 4-{(4-cyclohex-1-enylbenzyl)-[3-(3-trifluoromethoxyphenyl)acryloyl]amino}benzoic acid methy ester.

HPLC-MS (Method (B)): m/z: 536 (M+1), Rt: 6.53 min.

4-{(4-Cyclohex-1-enylbenzyl)-[3-(3-trifluoromethoxyphenyl)acryloyl]amino}benzoic acid

A solution of 4-{(4-cyclohex-1-enylbenzyl)-[3-(3-trifluoromethoxyphenyl)acryloyl]amino}benzoic acid methy ester (0.68 g, 1.27 mmol) in THF (10 mL) and methanol (10 mL) was stirred and 1 M NaOH was added (3.8 mL). The mixture was stirred for 16 hours at room temperature and then acidified (pH=5) with 1 M hydrochloric acid. The organic solvents were removed under removed pressure and water was added. The precipitate was filtered off, washed with water and finally petroleum ether. The off white solid was dried in vacuo at 35° C. to afford 0.58 g (88%) of 4-{(4-cyclohex-1-enylbenzyl)-[3-(3-trifluoromethoxyphenyl)acryloyl]amino}benzoic acid.

HPLC-MS (Method (B): m/z: 522 (M+1), Rt: 5.61 min.

3-(4{(4-Cyclohex-1-enyl benzyl)-[3-(3-trifluoromethoxyphenyl)acryloyl]-amino}-benzoylamino)propionic acid methyl ester

A solution of 4-{(4-cyclohex-1-enylbenzyl)-[3-(3-trifluoromethoxyphenyl)acryloyl]-amino}benzoic acid (0.3 g; 0.57 mmol) in 2 mL of DMF and 5 mL of DCM was stirred while 1-hydroxybenzotriazole hydrate (0.093 g; 0.69 mmol) and EDAC (0.132 g; 0.69 mmol), were added. The mixture was stirred for 30 minutes at room temperature and methyl 3-aminopropionate hydrochloride (0.104 g; 0.75 mmol) and DIPEA (0.30 mL; 1.73 mmol) were added. The mixture was stirred at 40° C. for 3 hours and evaporated under reduced pressure. The residue was partitioned between water and ether. The organic phase was separated, washed twice with water and evaporated to afford 0.35 g (100%) of 3-(4{(4-cyclohex-1-enylbenzyl)-[3-(3-trifluoromethoxyphenyl)acryloyl]-amino}benzoylamino)propionic acid methyl ester.

HPLC-MS (Method (B)): m/z: 607 (M+1), Rt: 5.73 min.

3-(4{(4-Cyclohex-1-enylbenzyl)-[3-(3-trifluoromethoxyphenyl)acryloyl]amino}-benzoylamino)propionic acid

3-(4{(4-cyclohex-1-enylbenzyl)-[3-(3-trifluoromethoxyphenyl)acryloyl]-amino}benzoylamino)propionic acid methyl ester (0.35 g, 0.57 mmol) was dissolved in a mixture of methanol (10 mL) and THF (10 mL) and 1 M sodium hydroxide (1.7 mL, 1.71 mmol) was added. The mixture was stirred for 16 hours at room temperature and concentrated to an oil. The pH was adjusted to 4 by addition of 1 M hydrochloric acid and the solution was concentrated to an oil. The oil was purified by chromatography to afford 0.19 g (56%) of the title compound.

M.p. 178-180° C. HPLC-MS (Method (B)): m/z: 593 (M+1), Rt: 5.28 min.

The following examples were made as described above.

### Example 212

### General Procedure (I)

3-(4-{(4-Cyclohex-1-enylbenzyl)-[2-(3,4-dichlorophenyl)acetyl]amino}benzoylamino)propionic acid

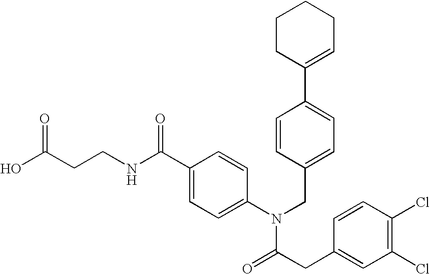

HPLC-MS (Method (B)): m/z: 565 (M+1), Rt: 5.15 min.

### Example 213

### General Procedure (I)

3-(4-{(4-Cyclohex-1-enylbenzyl)-[2-(4-trifluoromethoxyphenyl)acetyl]amino}benzoylamino]propionic acid

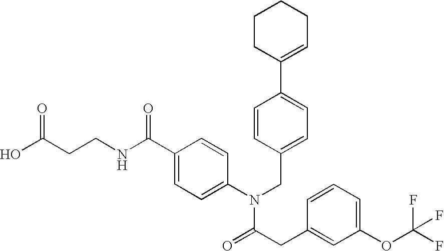

HPLC-MS (Method (B)): m/z: 581 (M+1), Rt: 5.12 min.

Pharmacological Methods

In the following section binding assays as well as functional assays useful for evaluating the efficiency of the compounds of the invention are described.

Binding of compounds to the glucagon receptor may be determined in a competition binding assay using the cloned human glucagon receptor.

Antagonism may be determined as the ability of the compounds to inhibit the amount of cAMP formed in the presence of 5 nM glucagon.

Glucagon Binding Assay (I)

Receptor binding are assayed using cloned human receptor (Lok et al., Gene 140, 203-209 (1994)). The receptor inserted in the pLJ6′ expression vector using EcoRI/SSt1 restriction sites (Lok et al.) is expressed in a baby hamster kidney cell line (A3 BHK 570-25). Clones are selected in the presence of 0.5 mg/ml G418 and are shown to be stable for more than 40 passages. The K_{d} is shown to be 0.1 nM.

Plasma membranes are prepared by growing cells to confluence, detaching them from the surface and resuspending the cells in cold buffer (10 mM tris/HCl, pH 7.4 containing 30 mM NaCl, 1 mM dithiothreitol, 5 mg/l leupeptin (Sigma), 5 mg/l pepstatin (Sigma), 100 mg/l bacitracin (Sigma) and 15 mg/l recombinant aprotinin (Novo Nordisk A/S)), homogenization by two 10-s bursts using a Polytron PT 10-35 homogenizer (Kinematica), and centrifugation upon a layer of 41 w/v % sucrose at 95.000×g for 75 min. The white band located between the two layers is diluted in buffer and centrifuged at 40.000×g for 45 min. The precipitate containing the plasma membranes is suspended in buffer and stored at −80° C. until use.

Glucagon is iodinated according to the chloramine T method (Hunter and Greenwood, Nature 194, 495 (1962)) and purified using anion exchange chromatography (Jørgensen et al., Hormone and Metab. Res. 4, 223-224 (1972). The specific activity is 460 μCi/μg on the day of iodination. Tracer is stored at −18° C. in aliquots and used immediately after thawing.

Binding assays are carried out in triplicate in filter microtiter plates (MADV N65, Millipore). The buffer is 50 mM HEPES, 5 mM EGTA, 5 mM MgCl₂, 0.005% tween 20, pH 7.4. Glucagon is dissolved in 0.05 M HCl, added an equal amount (w/w) of human serum albumin and freeze-dried. On the day of use, it is dissolved in water and diluted in buffer to the desired concentrations.

Test compounds are dissolved and diluted in DMSO. 140 μl buffer, 25 μl glucagon or buffer, and 10 μl DMSO or test compound are added to each well. Tracer (50.000 cpm) is diluted in buffer and 25 μl is added to each well. 1-4 μg freshly thawed plasma membrane protein diluted in buffer is then added in aliquots of 25 μl to each well. Plates are incubated at 30° C. for 2 hours. Non-specific binding is determined with 10⁻⁶ M of glucagon. Bound tracer and unbound tracer are then separated by vacuum filtration (Millipore vacuum manifold). The plates are washed with 2×100 μl buffer/well. The plates are air dried for a couple of hours, whereupon the filters are separated from the plates using a Millipore Puncher. The filters are counted in a gamma counter.

Functional Assay (I)

The functional assay is carried out in 96 well microtiter plates (tissue culture plates, Nunc). The resulting buffer concentrations in the assay are 50 mM tris/HCl, 1 mM EGTA, 1.5 mM MgSO₄, 1.7 mM ATP, 20 μM GTP, 2 mM IBMX, 0.02% tween-20 and 0.1% human serum albumin. pH was 7.4. Glucagon and proposed antagonist are added in aliquots of 35 μl diluted in 50 mM tris/HCl, 1 mM EGTA, 1.85 mM MgSO₄, 0.0222% tween-20 and 0.111% human serum albumin, pH 7.4.20 μl of 50 mM tris/HCl, 1 mM EGTA, 1.5 mM MgSO₄, 11.8 mM ATP, 0.14 mM GTP, 14 mM IBMX and 0.1% human serum albumin, pH 7.4 was added. GTP was dissolved immediately before the assay.

50 μl containing 5 μg of plasma membrane protein was added in a tris/HCl, EGTA, MgSO₄, human serum albumin buffer (the actual concentrations are dependent upon the concentration of protein in the stored plasma membranes).

The total assay volume is 140 μl. The plates are incubated for 2 hours at 37° C. with continuous shaking. Reaction is terminated by addition of 25 μl 0.5 N HCl. cAMP is measured by the use of a scintillation proximity kit (Amersham).

Glucagon Binding Assay (II)

BHK (baby hamster kidney cell line) cells are transfected with the human glucagon receptor and a membrane preparation of the cells is prepared. Wheat Germ Agglutinin derivatized SPA beads containing a scintillant (WGA beads) (Amersham) bound the membranes. ¹²⁵I-glucagon bound to human glucagon receptor in the membranes and excited the scintillant in the WGA beads to light emission. Glucagon or samples binding to the receptor competed with ¹²⁵I-glucagon.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The glucagon binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl glucagon or test compound (in DMSO) are added to each well. 50 μl tracer (¹²⁵I-porcine glucagon, 50.000 cpm) and 50 μl membranes (7.5 μg) containing the human glucagon receptor are then added to the wells. Finally 50 μl WGA beads containing 1 mg beads are transferred to the well. The opti plates are incubated for 4 hours on a shaker and then settled for 8-48 hours. The opti plates are counted in a Topcounter. Non-specific binding is determined with 500 nM of glucagon.

Most of the compounds according to the examples showed IC₅₀ values below 1000 nM when tested in the glucagon binding assay (II).

GIP Binding Assay

BHK (baby hamster kidney cell line) cells are transfected with the human GIP receptor and a membrane preparation of the cells is prepared. Wheat Germ Agglutinin derivatized SPA beads containing a scintillant (WGA beads) (Amersham) bound the membranes. ¹²⁵I-GIP bound to human GIP receptor in the membranes and excited the scintillant in the WGA beads to light emission. GIP or samples binding to the receptor competed with ¹²⁵I-GIP.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The GIP binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl GIP or test compound (in DMSO) are added to each well. 50 μl tracer (¹²⁵I-porcine GIP, 50.000 cpm) and 50 μl membranes (20 μg) containing the human GIP receptor are then added to the wells. Finally 50 μl WGA beads containing 1 mg beads are transferred to the well. The opti plates are incubated for 3.5 hours on a shaker and then settled for 8-48 hours. The opti plates are counted in a Topcounter. Non-specific binding is determined with 500 nM of GIP.

Generally, the compounds show a higher affinity for the glucagon receptor compared to the GIP receptor.

1. A compound of the general formula (I) 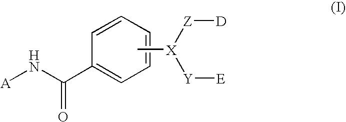
wherein
A is 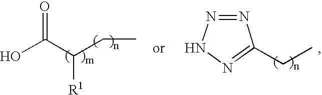
m is 0 or 1,
n is 0, 1, 2 or 3,
with the proviso that m and n must not both be 0,
R¹ is hydrogen, fluoro or —(CH₂)₀—OR²,
o is 0 or 1,
R² is hydrogen, C₁₋₆-alkyl, C₁₋₆-alkanoyl, aryl or aryl-C₁₋₆-alkyl,
X is N, CH or C with a double bond to one substituent,
Z is —CR³R⁴—, —(C═O)—(NR⁵)—(C₁₋₆-alkyl)_{K}—, —(C═O)—O—(C₁₋₆-alkyl)_{K}—, —(C═O)—(C₁₋₆-alkyl)_{K}—, —(C₁₋₆-alkyl)_{K}(C═O)—O—, —(C═O)—O—(C₂₋₆-alkenyl)_{K}—, —(C═O)—(C₂₋₆-alkenyl)_{K}—, —(C₁₋₆-alkenyl)_{K}(C═O)—O—
wherein k is 0 or 1,
R³, R⁴ and R⁵ are independently selected from hydrogen, C₁₋₁-alkyl or aryl,
Y is —C₁₋₆-alkyl)ₛ—(C═O)—(C₁₋₆-alkyl)ₜ—, —C₁₋₆-alkenyl)ₙ—(C═O)—(C₁₋₆-alkyl)ₜ—, —C₁₋₆-alkyl-, —C₂₋₆-alkenyl-, or —CR⁶R⁷—
wherein s and t independently are 0 or 1;
wherein R⁶, R⁷ and R⁸ independently are selected from hydrogen, C₁₋₆-alkyl and aryl;
D is aryl or heteroaryl, which may optionally be substituted with one or more substituents R¹⁶, R¹⁷ R¹⁸, R¹⁹, R²⁰ and R², wherein
R¹⁶, R¹⁷, R¹⁸ and R¹⁹ independently are
hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR²², —NR²²R²³, —SR²², —NR²²S(O)₂R²³, —S(O)₂NR²²R²³, —S(O)NR²²R²³, —S(O)R²², —S(O)₂R²²—C(O)NR^{R}, —OC(O)NR²²R²³, —NR²²C(O)R²³, —CH₂C(O)NR²²R²³, —OCH₂C(O)NR²²R²³, —CH₂OR²², —CH₂NR²² R²³, —OC(O)R²², —C(O)R²² or —C(O)OR²²,
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²², —NR²²R²³ and C₁₋₆-alkyl,
C₃₋₈-cycloalkyl, C₄₋₈-Cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-Cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl, aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties optionally may be substituted with one or more substituents selected from halogen, —C(O)OR²², —CN, —CF₃, —OCF₃, —NO₂, —OR²², —NR²²R²³ and C₁₋₆-alkyl,
R²² and R²³ independently are hydrogen, C₁₋₆-alkyl, aryl-C₁₋₆-alkyl or aryl, or R²² and R²³ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹⁶ to R¹⁹ when placed in adjacent positions together may form a bridge —(CR²⁴R²⁵)ₐ—O—(CR²⁶R²⁷)_{c}—O—,
a is 0, 1 or 2,
c is 1 or 2,
R²⁴, R²⁵, R²⁶ and R²⁷ independently are hydrogen, C₁₋₆-alkyl or fluoro,
R²⁰ and R²¹ independently are hydrogen, C₁₋₆-alkyl, C₃₋₈-cycloalkyl or C₃₋₈-cycloalkyl-C₁₋₆-alkyl,
E is
C₃₋₈-cycloalkyl or C₄₋₈-cycloalkenyl, which may optionally be substituted with one or two substituents R²⁸ and R²⁹, which are independently selected from
hydrogen, halogen, —CN, —CF₃, —OR³³, —NR³³R³⁴, C₁₋₆-alkyl, C₃₋₈-cycloalkyl, C₄₋₈-cycloalkenyl, heteroaryl and aryl,
wherein the heteroaryl and aryl groups optionally may be substituted with one or more substituents selected from halogen, —CN, —CF₃, —NO₂, —OR³³, —NR³³R³⁴ and C₁-alkyl,
R³³ and R³⁴ independently are hydrogen or C₁₋₆-alkyl,
or R³³ and R³⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
aryl, heteroaryl, aryl-C₂₋₆-alkenyl or aryl-C₂₋₆-alkynyl, of which the cyclic moieties may optionally be substituted with one to three substitutents R³⁰, R³′ and R³², which are independently selected from
hydrogen, halogen, —CHF₂, —CF₃, —OCF₃, —OCHF₂, —OCH₂CF₃, —OCF₂CHF₂, —SCF₃, —OR³⁵, —NR³⁵R³⁶, —SR³⁵, —S(O)R³⁵, —S(O)₂R³⁵, —C(O)NR³⁵R³⁶, —OC(O)NR³⁵R³, —NR³⁵C(O)R³⁶, —OCH₂C(O)NR³⁵R³⁶, —C(O)R³⁵ and —C(O)OR³⁵,
C₁₋₆-alkyl, C₂₋₆-alkenyl and C₂₋₈-alkynyl,
which may optionally be substituted with one or more substituents selected from halogen, —CN, —CF₃, —OCF₃, —SCF₃, —NO₂, —OR³⁵, —NR³⁵R³⁶ and C₁₋₄alkyl,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl, aryl, aryloxy, aroyl, aryl-C₁₋₆-alkoxy, aryl-C₁₋₆alkyl, aryl-C₂₋₆-alkenyl, aryl-C₂₋₆-alkynyl, heteroaryl, heteroaryl-C₁₋₆-alkyl, heteroaryl-C₂₋₆-alkenyl and heteroaryl-C₂₋₆-alkynyl,
of which the cyclic moieties optionally may be substituted with one or more substituents selected from halogen, —CN, —CF₃, —OCF₃, —SCF₃, —NO₂, —OR³⁵, —NR³⁵R³⁶ and C₁₋₆-alkyl,
wherein R³⁵ and R³⁶ independently are hydrogen, C₁₋₆-alkyl or aryl,
or R³⁵ and R³⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the substituents R³⁰, R³¹ and R³² when attached to the same ring carbon atom or different ring carbon atoms together may form a radical —O—(CH₂)ₜ—CR³⁷ R³—(CH₂)ₗ—O—, —CH₂)ₜ—CR³⁷R³⁸—(CH₂)ₗ— or —S—(CH₂)ₜ—CR³⁷R³⁸—(CH₂)ₗ—S—,
t and l independently are 0, 1, 2, 3, 4 or 5,
R³⁷ and R³⁸ independently are hydrogen or C₁₋₆-alkyl,
as well as any diastereomer or enantiomer or tautomeric form or mixture thereof, or a pharmaceutically acceptable salt thereof.

2. A compound according to claim 1, wherein A is

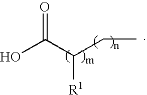

3. A compound according to claim 2, wherein A is

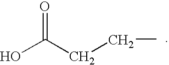

4. A compound according to claim 2, wherein A is

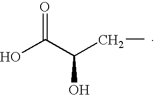

5. A compound according to claim 1, wherein A is

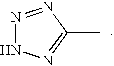

6. A compound according to claim 1, wherein D is

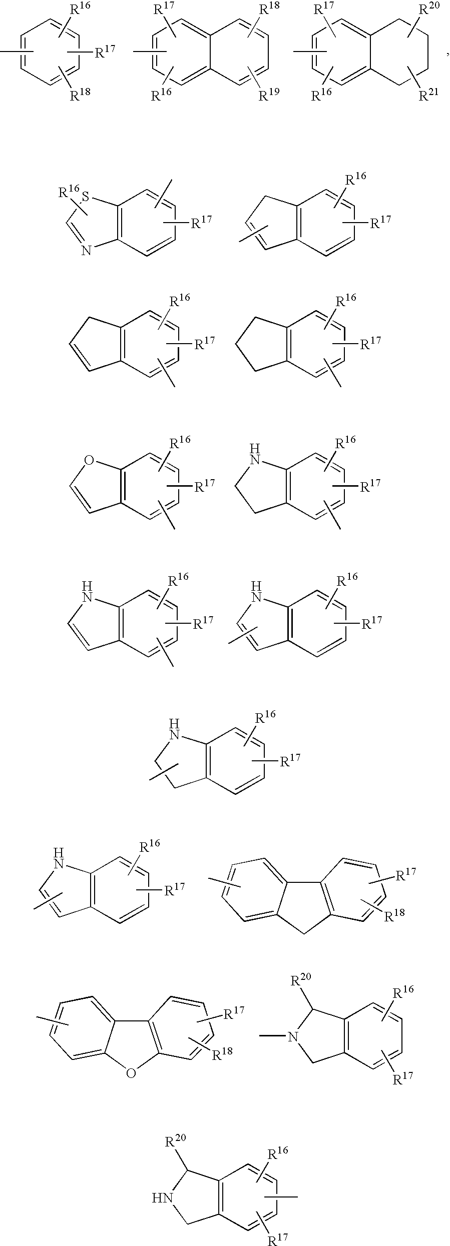

7. A compound according to claim 6, wherein D is

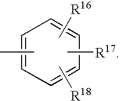

8. A compound according to claim 6, wherein R¹⁶, R¹⁷ and R¹⁸ independently are
hydrogen, halogen, —CN, —CH₂CN, —CHF₂, —CF₃, —OCF₃, —OCHF₂, —OCH₂CF₃, —OCF₂CHF₂, —S(O)₂CF₃, —SCF₃, —NO₂, —OR²², —NR²²R²³, —SR²², —NR²²S(O)₂R²³, —S(O)₂NR²²R²³, —S(O)NR²²R²³, —S(O)R²²—S(O)₂R²², —C(O)NR²²R²³, —OC(O)NR²²R²³, —NR²²C(O)R²³, —CH₂C(O)NR²²R²³, —OCH₂C(O)NR²²R²³, —CH₂OR²², —CH₂NR²²R²³, —OC(O)R²², —C(O)R²² or —C(O)OR²²,
C₁₋₆ alkyl, which may optionally be substituted with one or more substituents selected from fluoro, —CN, —CF₃, —OCF₃, —OR²² and —NR²²R²³,
C₃₋₈-cycloalkyl, which may optionally be substituted with one or more substituents selected from fluoro, —C(O)OR²⁴, —CN, —CF₃, —OCF₃, —OR²², —NR²²R²³ and C₁₋₄alkyl,
aryl or aryloxy, which may optionally be substituted with one or more substituents selected from halogen, —C(O)OR²², —CN, —CF₃, —OCF₃, —NO₂, —OR²², —NR²²R²³ and C₁₋₆-alkyl,
R²² and R²³ independently are hydrogen, C₁₋₆-alkyl, aryl-C₁-alkyl or aryl, or R²² and R²³ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹⁶ to R¹⁸ when placed in adjacent positions together may form a bridge —CR²⁴R²⁵)ₐ—O—(CR²⁶R²⁷)_{c}—O—,
a is 0, 1 or 2,
c is 1 or 2,
R²⁴, R²⁵, R²⁶ and R²⁷ independently are hydrogen, C₁₋₆-alkyl or fluoro.

9. A compound according to claim 8, wherein R¹⁶, R¹⁷ and R¹⁸ independently are
hydrogen, halogen, CN, —CF₃, —OCF₃, —SCF₃, —S(O)C₁₋₆-alkyl-, —C(O)C₁₋₆-alkyl-, C₁₋₆-alkyl, C₁₋₆-alkoxy, phenyl, cyclopentyl, cyclohexyl or phenoxy,
or two of the groups R¹⁶ to R¹⁸ when placed in adjacent positions together may form a bridge —O—(CF₂)₂—O—, —CF₂—O—CF₂—O— or —O—CH₂—O—.

10. A compound according to claim 1, wherein E is

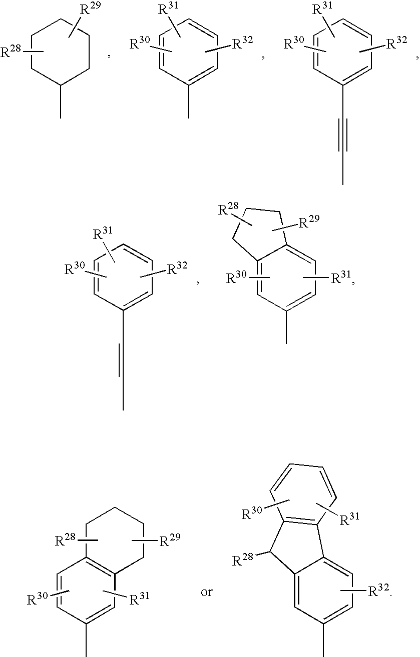

11. A compound according to claim 10, wherein E is

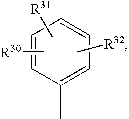

12. A compound according to claim 10, wherein R³⁰, R³¹ and R³² independently are
hydrogen,
halogen, —OCF₃, —SCF₃, —OCHF₂ or —CF₃,
C₁₋₆-alkyl, which may optionally be substituted with one or more substituents selected from fluoro, —CN, —CF₃, —OCF₃, —OR³⁵ and —NR³⁵R³⁶,
C₃₋₈-cycloalkyl or C₄₋₈-cycloalkenyl, which may optionally be substituted with one or more substituents selected from fluoro, —CN, —CF₃, —OCF₃, —OR³⁵, —NR³⁵R³⁶ and C₁₋₆-alkyl,
aryl, heteroaryl, aryloxy or aryl-C₁₋₆-alkoxy, of which the aryl moieties may optionally be substituted with one or more substituents selected from halogen, —CN, —CF₃, —OCF₃, —NO₂, —R³⁵, —NR³⁵R³⁶ and C₁₋₆-alkyl,
R³⁵ and R³⁶ independently are hydrogen, C₁₋₁-alkyl or aryl,
or R³⁵ and R³⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

13. A compound according to claim 12, wherein R³⁰, R³¹ and R³² independently are
hydrogen,
halogen, —OCF₃, —OCHF₂, —SCF₃, or —CF₃,
C₁₋₆-alkyl, which may optionally be substituted with one or more substituents selected from fluoro, —CN, —CF₃, —OCF₃, —OR³⁵ and —NR³⁵R³⁵,
cyclohexyl or cyclohex-1-enyl, which may optionally be substituted with one or more substituents selected from fluoro, —CN, —CF₃, —OCF₃, —OR³⁵, —NR³⁵R³⁶ and C₁₋₆-alkyl,
phenyl which may optionally be substituted with one or more substitutents selected from halogen, —CN, —CF₃, —OCF₃, —NO₂, —OR³⁵, —NR³⁵R³⁶ and C₁₋₆-alkyl,
phenoxy or benzyloxy, of which the phenyl moieties may optionally be substituted with one or more substituents selected from halogen, —CN, —CF₃, —OCF₃, —NO₂, —OR³⁵, —NR³⁵R³⁶ and C₁₋₆-alkyl,
thiadiazolyl,
R³⁵ and R³⁶ independently are hydrogen or C₁₋₆-alkyl.

14. A compound according to claim 10, wherein R³⁰ and R³² are both hydrogen, and R³¹ is different from hydrogen.

15. A compound according to claim 1, wherein Y is —C═O—, —CH₂—.

16. A compound according to claim 1, wherein Z is —CH₂—, —(C═O)—(NH), —(C═O)—O— or —(C═O)—CH₂—.

17. A compound of general formula (Ia); 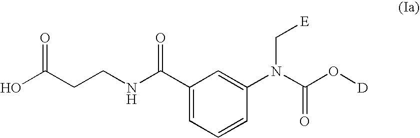
wherein D is selected from the following: 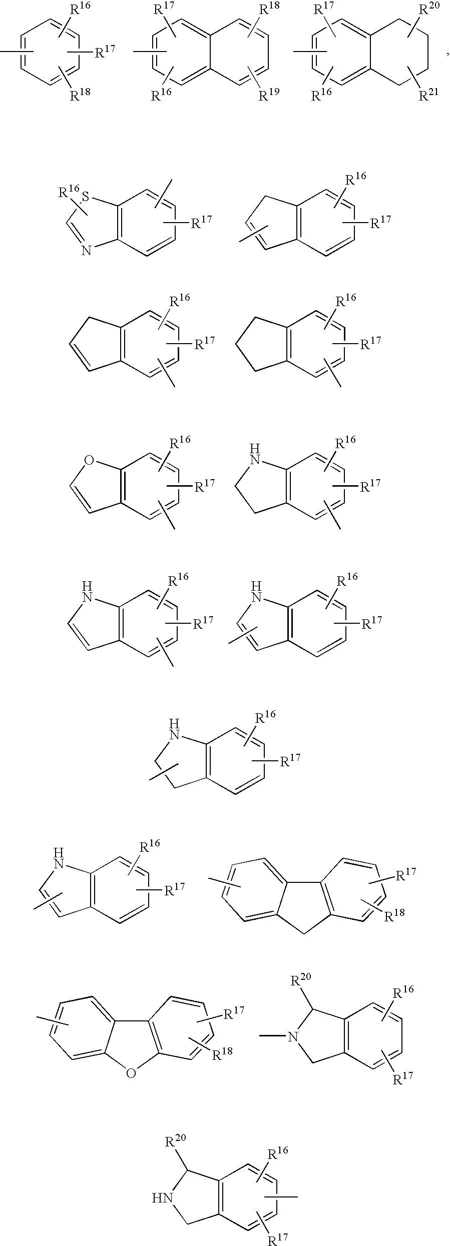
and wherein E is selected from the following: 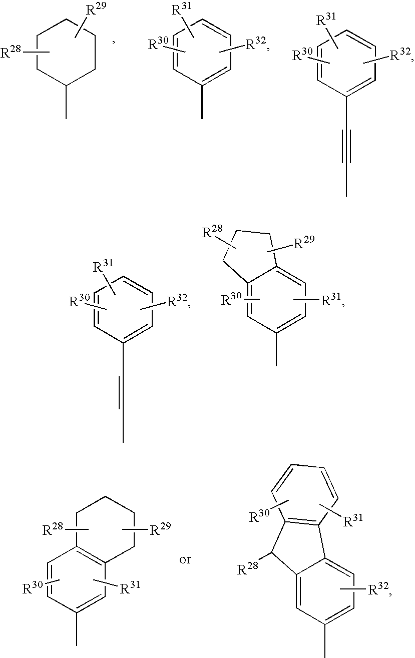
as well as any diastereomer or enantiomer or tautomeric form, or mixture thereof, or pharmaceutical acceptable salts thereof.

18. A compound of general formula (Ib); 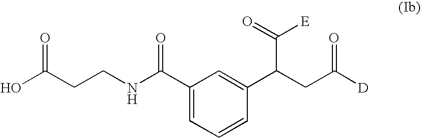
wherein D is selected from the following, 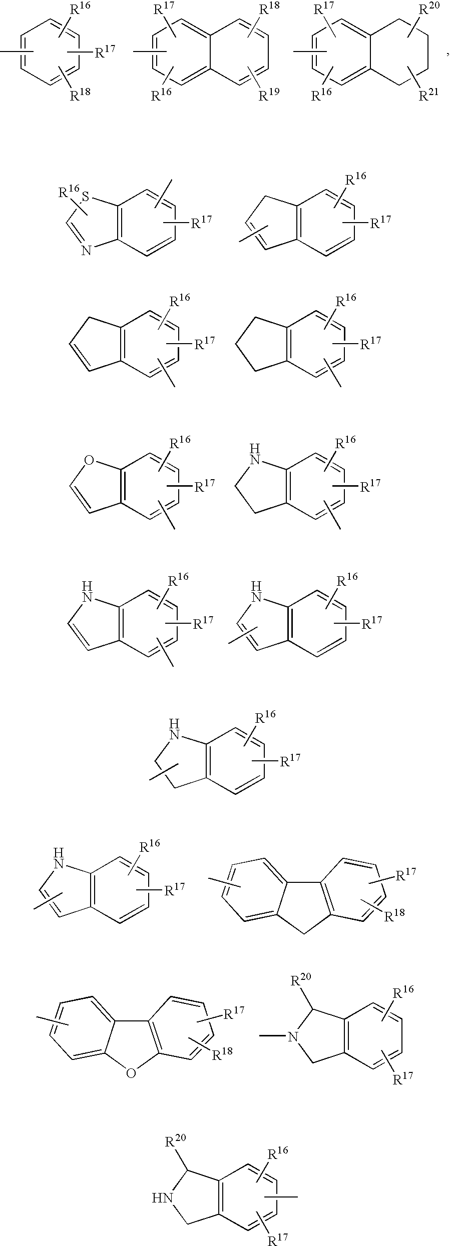
and wherein E is selected from the following: 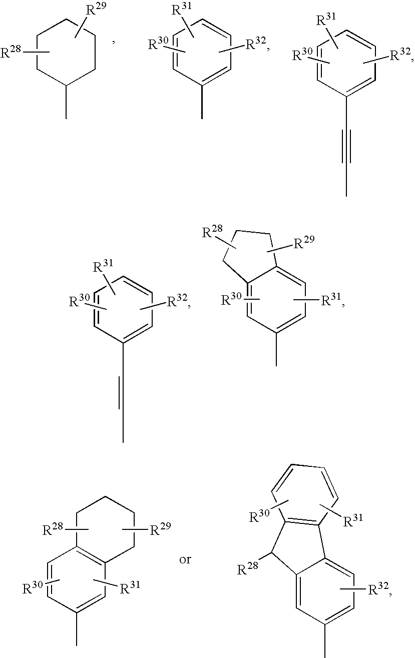
as well as any diastereomer or enantiomer or tautomeric form thereof including mixtures of these or pharmaceutical acceptable salts thereof.

19. A compound of general formula (Ic) 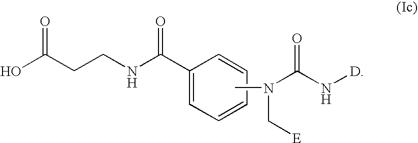
wherein D is selected from the following, 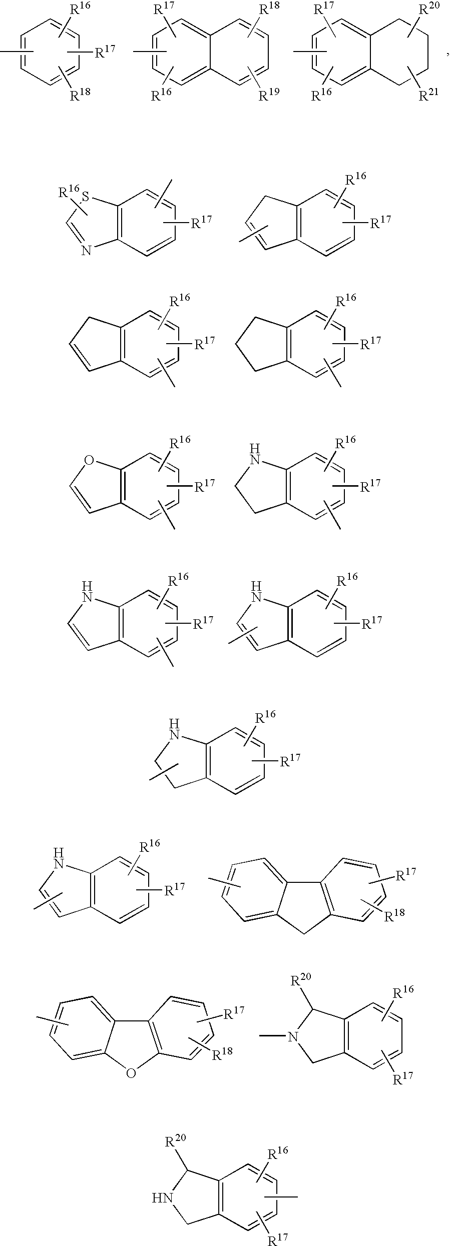
and wherein E is selected from the following, 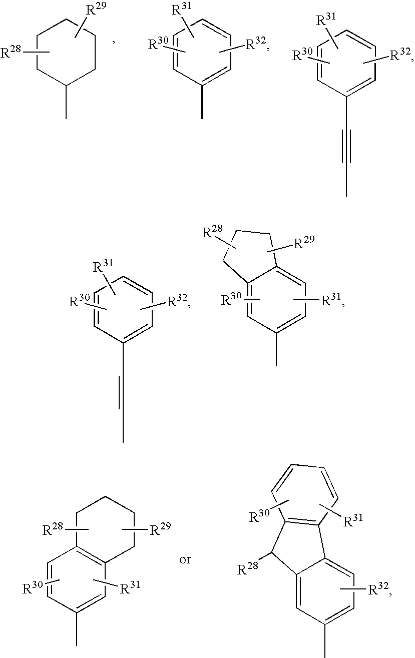
as well as any diastereomer or enantiomer or tautomeric form thereof including mixtures of these or pharmaceutical acceptable salts thereof.

20. A compound of the general formula (Id): 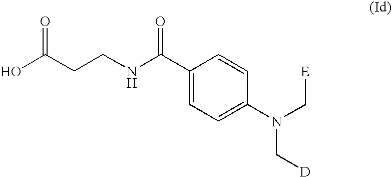
wherein D is selected from the following: 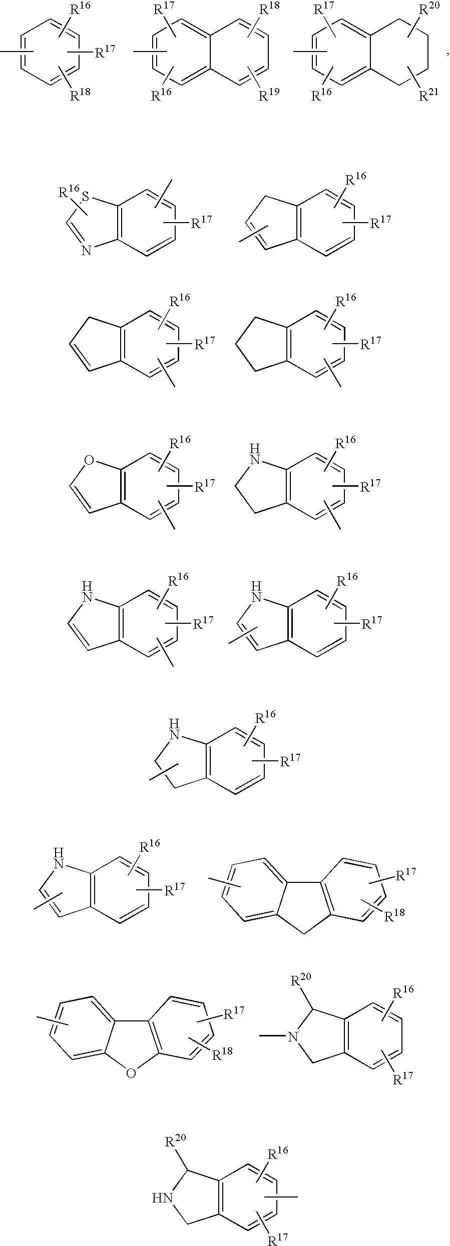
and wherein E is selected from the following: 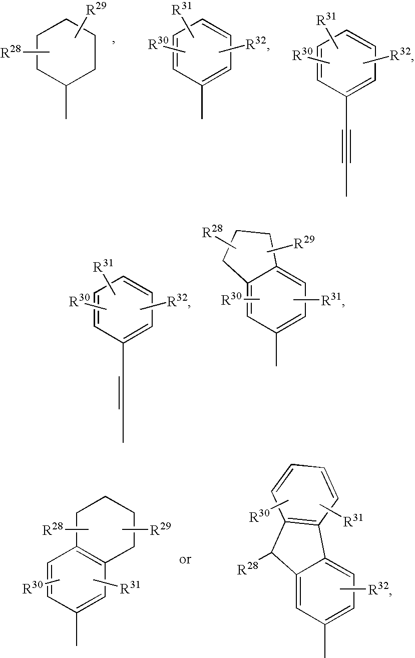
as well as any diastereomer or enantiomer or tautomeric form thereof including mixtures of these or pharmaceutical acceptable salts thereof.

21. A compound according to claim 1, which has an IC₅₀ value of no greater than 5 μM as determined by the Glucagon Binding Assay (I) or Glucagon Binding Assay (II) disclosed herein.

22. A compound according to claim 21, which has an IC₅₀ value of less than 1 μM, preferably of less than 500 nM and even more preferred of less than 100 nM as determined by the Glucagon Binding Assay (I) or Glucagon Binding Assay (II) disclosed herein.

23. A compound according to claim 1, which is an agent useful for the treatment of an indication selected from the group consisting of hyperglycemia, IGT, type 2 diabetes, type 1 diabetes, dyslipidemia and obesity.

24. A pharmaceutical composition comprising, as an active ingredient, at least one compound according to claim 1 together with one or more pharmaceutically acceptable carriers or excipients.

25. A pharmaceutical composition according to claim 24 in unit dosage form, comprising from about 0.05 mg to about 1000 mg, preferably from about 0.1 mg to about 500 mg and especially preferred from about 0.5 mg to about 200 mg of the compound.

26. A method of treating disorders or diseases, wherein a glucagon antagonistic action is beneficial, comprising administering to a subject in need thereof a therapeutically effective amount of a compound of claim 1.

27. A method of treating glucagon-mediated disorders and diseases, comprising administering to a subject in need thereof a therapeutically effective amount of a compound of claim 1.

28. A method of treating hyperglycemia, comprising administering to a subject in need thereof a therapeutically effective amount of a compound of claim 1.

29. A method of lowering blood glucose in a mammal, comprising administering to the mammal in need of such treatment a therapeutically effective amount of a compound of claim 1.

30. A method of treating IGT, comprising administering to a subject in need thereof a therapeutically effective amount of a compound of claim 1.

31. A method of treating type two diabetes, comprising administering to a subject in need thereof a therapeutically effective amount of a compound of claim 1.

32. A method of delaying or prevention of the progression from non-insulin requiring type 2 diabetes to insulin requiring type two diabetes in a subject, comprising administering to a subject in need thereof a therapeutically effective amount of a compound of claim 1.

33. A method of delaying or prevention of the progression from IGT to type 2 diabetes in a subject, comprising administering to a subject in need thereof a therapeutically effective amount of a compound of claim 1.

34. A method of treating type one diabetes, comprising administering to a subject in need thereof a therapeutically effective amount of a compound of claim 1.

35. A method of treating obesity, comprising administering to a subject in need thereof a therapeutically effective amount of a compound of claim 1.

36. A method of treating dyslipidemia, comprising administering to a subject in need thereof a therapeutically effective amount of a compound of claim 1.

37. A method according to claim 26 wherein the subject is administered a further antidiabetic agent.

38. A method according to claim 26 wherein the subject is administered a further antiobesity agent.

39. A method according to claim 26 wherein the subject is administered a further antihyperlipidemic agent.

40. A method according to claim 26 wherein the subject is administered a further antihypertensive agent.

41. A method of treating disorders or diseases, wherein a glucagon antagonistic action is beneficial, comprising administering to a subject in need thereof a therapeutically effective amount of a pharmaceutical composition of claim 24.

42. The method according to claim 26, wherein the effective amount of the compound is in the range of from about 0.05 mg to about 2000 mg, preferably from about 0.1 mg to about 1000 mg and especially preferred from about 0.5 mg to about 500 mg per day.
